(19) United States
(12) Patent Application Publication
(10) Pub. No.: US 2026/0085066 A1
(43) Pub. Date: Mar. 26, 2026

(54) LEUCINE RICH REPEAT KINASE 2 (LRRK2) DEGRADING COMPOUNDS AND ASSOCIATED METHODS OF USE

(71) Applicant: Arvinas Operations, Inc., New Haven, CT (US)

(72) Inventors: Steven M. Sparks, Guilford, CT (US); Handing Dong, Madison, CT (US); Michael Berlin, Yardley, PA (US)

(21) Appl. No.: 19/109,277

(22) PCT Filed: Sep. 6, 2023
(86) PCT No.: PCT/US2023/073589

§ 371 Date: Mar. 6, 2025

Related U.S. Application Data
(60) Provisional application No. 63/374,851, filed on Sep. 7, 2022.

(51) Int. Cl. C07D 413/14; A61K 31/506; A61K 31/5377; A61K 45/06; C07D 401/14; C07D 405/14; C07D 471/10

(52) U.S. Cl. CPC C07D 413/14; A61K 31/506; A61K 31/5377; A61K 45/06; C07D 401/14; C07D 405/14; C07D 471/10

(57) ABSTRACT

The present disclosure relates to bifunctional compounds that cause the degradation of LRRK2; pharmaceutical compositions comprising the compounds; and methods of treating disorders associated with LRRK2, including Parkinson's Disease.

### CROSS-REFERENCE TO RELATED APPLICATIONS

This application claims priority to U.S. Provisional Application Ser. No. 63/374,851, filed Sep. 7, 2022, which is incorporated herein by reference in its entirety.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands that bind to these ligases. For example, since the discovery of nutlins, the first small molecule E3 ligase inhibitors, additional compounds have been reported that target E3 ligases.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism that has not been completely elucidated, cereblon ubiquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

An ongoing need exists in the art for effective treatments for disease associated with overexpression or aggregation of Leucine-rich repeat kinase 2 (LRRK2) or the overactivation of LRRK2, Leucine-rich repeat kinase 2 (LRRK2) is a member of the leucine-rich repeat kinase family. Catalytic activities of LRRK2 are associated with the kinase and GTPase domain, and LRRK2 is a heterodimer in its active form (Greggio E, et al. J Biol Chem 2008, 283:16906-16914). GTP binding is essential for kinase activity, and mutations that prevent GTP binding have been shown to ablate LRRK2 kinase activity (Ito G, et al. Biochemistry 2007, 46:1380-1388). Expression levels of LRRK2 are highest in immune cells, such as neutrophils, monocytes and B cells, lung, and kidney, with lower levels in the brain where it is expressed in dopaminergic neurons of the substantia nigra (West A B, et al. J Comp Neurol 2014, 522:2465-80).

There are several dominant gain-of-function pathogenic and characterized mutations to LRRK2 located either in the Roco domains (N1437H, R1441G/C/H, Y1699C) effecting GTP hydrolysis or in the kinase domain (G2019S and I2020T). The G2019S is the most common LRRK2 mutation linked to Parkinson's disease (PD), which is a progressive neurodegenerative disorder characterized by resting tremors, rigidity, decreased movement (bradykinesia), and postural instability. The histological hallmarks of PD include neurodegeneration of the dopaminergic neurons in the substantia nigra pars compacta as well as intracellular inclusions called Lewy bodies and neurites consisting of the aggregated form of the alpha-synuclein protein. G2019S is associated with 1-2% of all PD patients and causes an increase in kinase activity of 2-fold in vitro (West A B, et al. Proc Natl Acad Sci USA 2005, 102: 16842-47) and autophosphorylation at Ser1292 is increased 4-fold (Sheng Z, et al. Sci Transl Med 2012, 4:164ra161). Several of the above Parkinson disease-associated mutations (R1441C/G, Y1699C and I2020T) suppress phosphorylation of LRRK2 at Ser910 and Ser935, which in turn reduces LRRK2 association with 14-3-3 proteins, thought to represent an inactive form of LRRK2 (Nichols J, et al. Biochem J 2010, 430:393-404).

Furthermore, LRRK2 is linked to autosomal dominant inherited PD through a mutation within a region of chromosome 12, termed PARK8, which is linked to the LRRK2 gene (Funayama M, et al. Ann Neurol 2002, 51:296-301; Zimprich A, et al. Neuron 2004, 44:601-607; Paisan-Ruiz C, et al. Neuron 2004, 44:595-600).

Lewy bodies are the main histological hallmark of PD. Lewy bodies are composed primarily of alpha-synuclein aggregates, and mutations in alpha-synuclein that increase this aggregation also increase the risk of developing PD (Meade R M, et al. Mol Neurodegener 2019, 14. 29). Depletion of LRRK2 with ASOs and deletion of LRRK2 at a genomic level have been shown to reduce alpha-synuclein mediated pathology in mouse models of PD (Lin X, et al. Neuron 2009, 64:807-27). Mutations increasing LRRK2 activity, such as G2019S, increase the aggregation of alpha-synuclein in neurons and mouse models of PD. This increase was reversed with LRRK2 kinase inhibitors (Volpicelli-Daley L A, et al. J Neurosci. 2016 Jul. 13; 36(28):7415-27). There is some evidence to suggest that the G2019S mutant form of LRRK2 is resistant to inhibition by kinase inhibitors in the CNS, potentially reducing their disease modifying effect (Kelly K, et al. Exp Neurol. 2018 November; 309:1-13). Even though most cases of PD also have Lewy bodies upon post-mortem examination, Lewy bodies are not present in a high number of LRRK2 G2019S mutation associated PD cases (Kalia L V, et al. JAMA neurol 2015, 72:100-05). In addition to Lewy bodies being a common feature of PD, Tau pathology is also a major feature of LRRK2 mutation carriers at post-mortem (Henderson M X, et al. Acta Neuropathol Commun 2019, 7, 183).

LRRK2 is highly expressed in the immune system in neutrophils, monocytes and macrophages, as well as in brain microglia, and is a modulator of the intrinsic regulation of microglial activation and of lysosomal degradation processes (Ma et al. Hum Mol Genet. 2014 Feb. 1; 23(3):831-41). Prolonged activation of these immune cells through PD disease processes or mutations in LRRK2 could increase neuroinflammation and lead to a greater risk of developing PD and/or Tau pathology. Treatment with anti-TNF agents reduces the risk of developing PD by 78% in patients with inflammatory bowel disorder (Peter I, et al.: JAMA Neurol 2018), thereby demonstrating the strong linkage between inflammation and PD. In addition to PD, LRRK2 has been linked to other diseases such as cancer, leprosy, and Crohn's disease (Lewis P A, (2012). Sci Signal. 5(207), pe2).

An ongoing need exists in the art for effective treatments for LRRK2 related disease and disorders, e.g., idiopathic PD, LRRK2 mutation associated PD (e.g., PD associated with one or more LRRK2 activating mutations), primary tauopathies (e.g., supranuclear palsy (PSP) or corticobasal degeneration (CBD)), lewy body dementia, Crohn's Disease, Leprosy (e.g., Leprosy with type 1 inflammatory reactions), and/or neuroinflammation.

### SUMMARY

Provided herein are compounds that function to recruit LRRK2 protein or a mutated version thereof to an E3 ubiquitin ligase for targeted ubiquitination and subsequent proteasomal degradation. As such, these compounds are useful in the treatment of a variety of indications, including Parkinson's Disease (PD).

Also provided herein are pharmaceutical compositions comprising a compound described herein, or a pharmaceutically acceptable salt thereof, and a pharmaceutically acceptable carrier.

Also provided herein is a method of treating a disease, a disorder, or a symptom causally related to LRRK2 in a subject in need thereof comprising administering to the subject an effective amount of a compound provided herein.

### DETAILED DESCRIPTION

Provided herein are compounds that function to recruit LRRK2 protein or a mutated version thereof to an E3 ubiquitin ligase for targeted ubiquitination and subsequent proteasomal degradation.

As such, these compounds, as well as pharmaceutical compositions that comprise these compounds, are useful in the treatment of a variety of indications, including Parkinson's Disease.

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or +10%, including ±5%, 1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises alleviating the symptoms of Parkinson's disease.

As used herein, the term “prevent” or “prevention” means no disorder or disease development if none had occurred, or no further disorder or disease development if there had already been development of the disorder or disease. Also considered is the ability of one to prevent some or all the symptoms associated with the disorder or disease.

“Bioactive agent” refers to an agent, other than a compound according to the present disclosure, which is used in combination with a present compound as an agent with biological activity to assist in effecting an intended therapy, inhibition, and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

As used herein, the term “cell” is meant to refer to a cell that is in vitro, ex vivo, or in vivo. In some embodiments, an ex vivo cell can be part of a tissue sample excised from an organism such as a mammal. In embodiments, an in vitro cell can be a cell in a cell culture. In embodiments, an in vivo cell is a cell living in an organism such as a mammal.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the term “effective amount” refers to a nontoxic but sufficient amount of an agent to provide the desired biological result. That result may be reduction or alleviation of the signs, symptoms, or causes of a disease, or any other desired alteration of a biological system. An appropriate effective amount in any individual case may be determined by one of ordinary skill in the art using routine experimentation. As is well understood in the medical arts an effective amount of a compound of this disclosure will be at a reasonable benefit/risk ratio applicable to any medical treatment.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

### Compounds

Provided herein is a compound selected from:

| Ex | Structure |
|---|---|
| 1 | 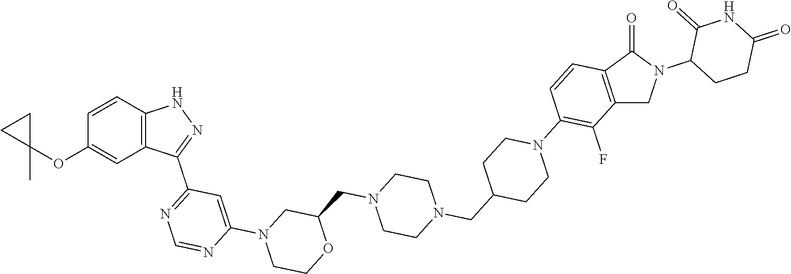 [SMILES: CC1(Oc2ccc3[nH]nc(-c4cc(N5CCO[C@H](CN6CCN(CC7CCN(c8ccc9c(c8F)CN(C8CCC(=O)NC8=O)C9=O)CC7)CC6)C5)ncn4)c3c2)CC1] |
| 2 | 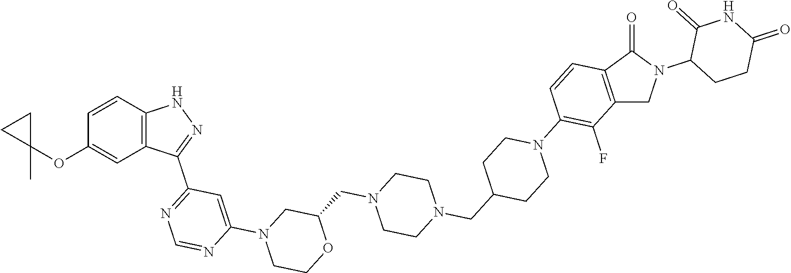 [SMILES: CC1(Oc2ccc3[nH]nc(-c4cc(N5CCO[C@@H](CN6CCN(CC7CCN(c8ccc9c(c8F)CN(C8CCC(=O)NC8=O)C9=O)CC7)CC6)C5)ncn4)c3c2)CC1] |
| 3 | 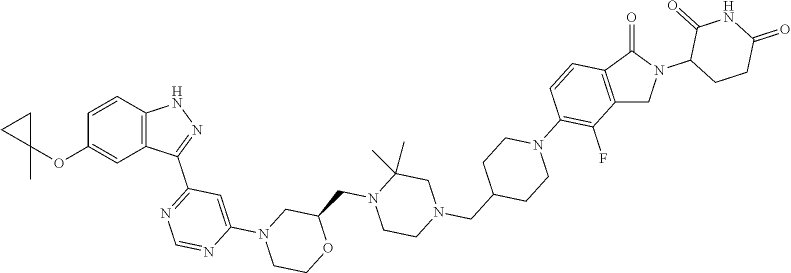 [SMILES: CC1(Oc2ccc3[nH]nc(-c4cc(N5CCO[C@H](CN6CCN(CC7CCN(c8ccc9c(c8F)CN(C8CCC(=O)NC8=O)C9=O)CC7)CC6(C)C)C5)ncn4)c3c2)CC1] |
| 4 | 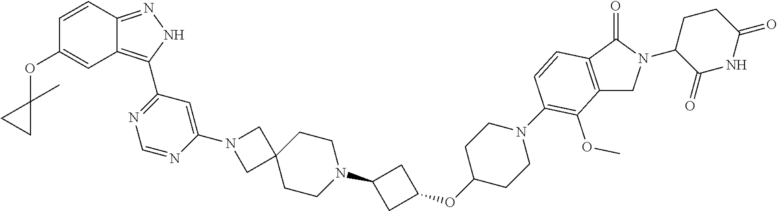 [SMILES: COc1c(N2CCC(O[C@H]3C[C@H](N4CCC5(CC4)CN(c4cc(-c6[nH]nc7ccc(OC8(C)CC8)cc67)ncn4)C5)C3)CC2)ccc2c1CN(C1CCC(=O)NC1=O)C2=O] |
| 5 | 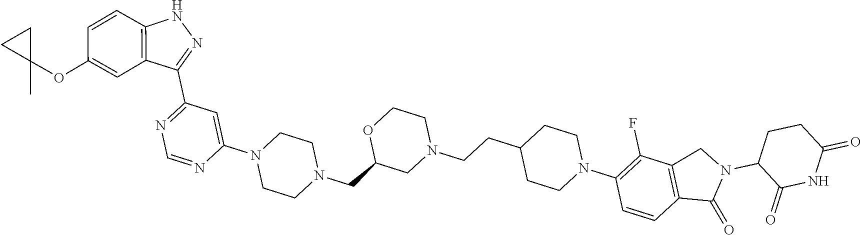 [SMILES: CC1(Oc2ccc3[nH]nc(-c4cc(N5CCN(C[C@@H]6CN(CCC7CCN(c8ccc9c(c8F)CN(C8CCC(=O)NC8=O)C9=O)CC7)CCO6)CC5)ncn4)c3c2)CC1] |
| 6 | 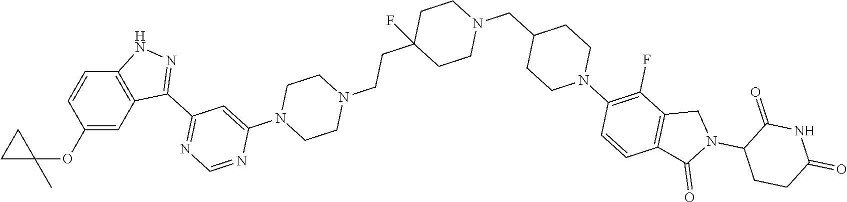 [SMILES: CC1(Oc2ccc3[nH]nc(-c4cc(N5CCN(CCC6(F)CCN(CC7CCN(c8ccc9c(c8F)CN(C8CCC(=O)NC8=O)C9=O)CC7)CC6)CC5)ncn4)c3c2)CC1] |
| 7 | 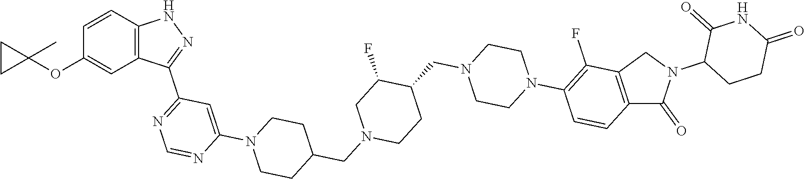 [SMILES: CC1(Oc2ccc3[nH]nc(-c4cc(N5CCC(CN6CC[C@@H](CN7CCN(c8ccc9c(c8F)CN(C8CCC(=O)NC8=O)C9=O)CC7)[C@@H](F)C6)CC5)ncn4)c3c2)CC1] |
| 8 | 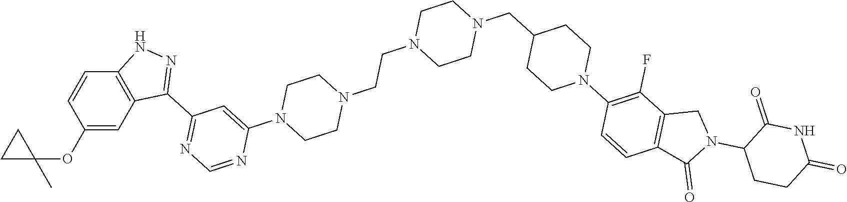 [SMILES: CC1(Oc2ccc3[nH]nc(-c4cc(N5CCN(CCN6CCN(CC7CCN(c8ccc9c(c8F)CN(C8CCC(=O)NC8=O)C9=O)CC7)CC6)CC5)ncn4)c3c2)CC1] |
| 9 | 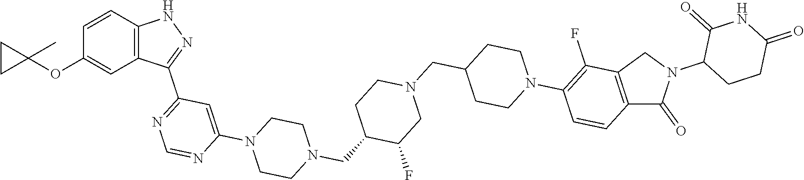 [SMILES: CC1(Oc2ccc3[nH]nc(-c4cc(N5CCN(C[C@@H]6CCN(CC7CCN(c8ccc9c(c8F)CN(C8CCC(=O)NC8=O)C9=O)CC7)C[C@@H]6F)CC5)ncn4)c3c2)CC1] |
| 10 | 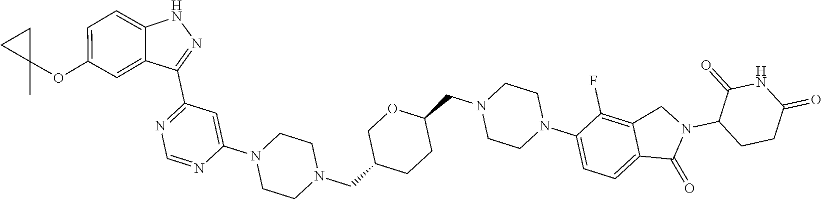 [SMILES: CC1(Oc2ccc3[nH]nc(-c4cc(N5CCN(C[C@H]6CC[C@H](CN7CCN(c8ccc9c(c8F)CN(C8CCC(=O)NC8=O)C9=O)CC7)OC6)CC5)ncn4)c3c2)CC1] |
| 11 | 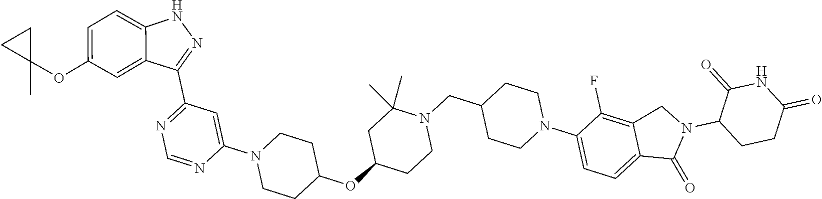 [SMILES: CC1(Oc2ccc3[nH]nc(-c4cc(N5CCC(O[C@@H]6CCN(CC7CCN(c8ccc9c(c8F)CN(C8CCC(=O)NC8=O)C9=O)CC7)C(C)(C)C6)CC5)ncn4)c3c2)CC1] |
| 12 | 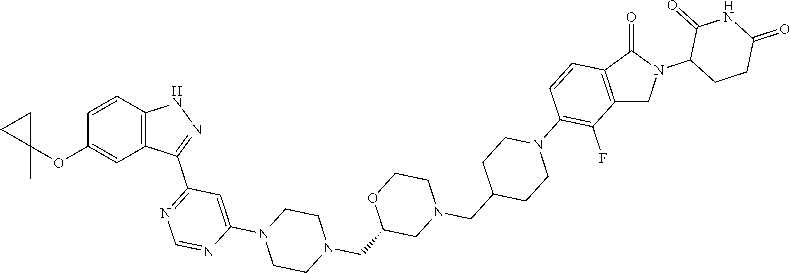 [SMILES: CC1(Oc2ccc3[nH]nc(-c4cc(N5CCN(C[C@H]6CN(CC7CCN(c8ccc9c(c8F)CN(C8CCC(=O)NC8=O)C9=O)CC7)CCO6)CC5)ncn4)c3c2)CC1] |
| 13 | 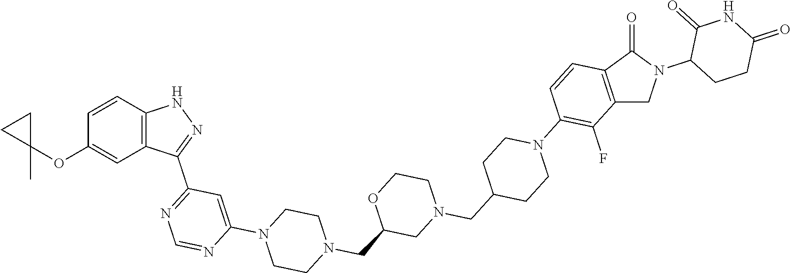 [SMILES: CC1(Oc2ccc3[nH]nc(-c4cc(N5CCN(C[C@@H]6CN(CC7CCN(c8ccc9c(c8F)CN(C8CCC(=O)NC8=O)C9=O)CC7)CCO6)CC5)ncn4)c3c2)CC1] |
| 14 | 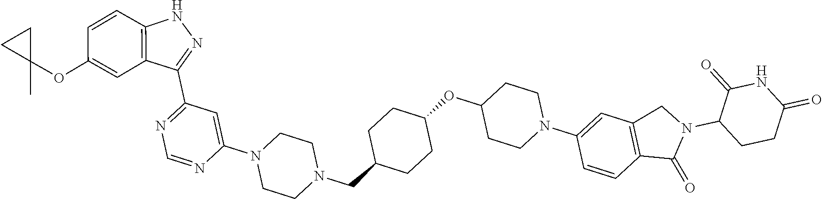 [SMILES: CC1(Oc2ccc3[nH]nc(-c4cc(N5CCN(C[C@H]6CC[C@H](OC7CCN(c8ccc9c(c8)CN(C8CCC(=O)NC8=O)C9=O)CC7)CC6)CC5)ncn4)c3c2)CC1] |
| 15 | 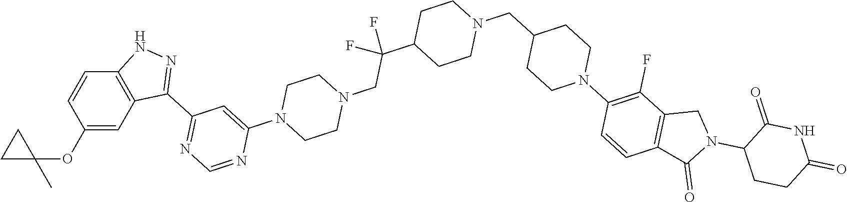 [SMILES: CC1(Oc2ccc3[nH]nc(-c4cc(N5CCN(CC(F)(F)C6CCN(CC7CCN(c8ccc9c(c8F)CN(C8CCC(=O)NC8=O)C9=O)CC7)CC6)CC5)ncn4)c3c2)CC1] |
| 16 | 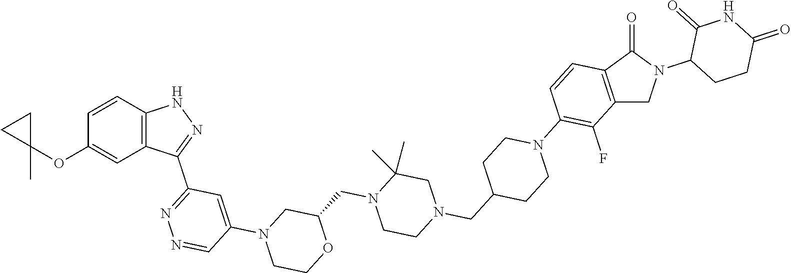 [SMILES: CC1(Oc2ccc3[nH]nc(-c4cc(N5CCO[C@@H](CN6CCN(CC7CCN(c8ccc9c(c8F)CN(C8CCC(=O)NC8=O)C9=O)CC7)CC6(C)C)C5)cnn4)c3c2)CC1] |
| 17 | 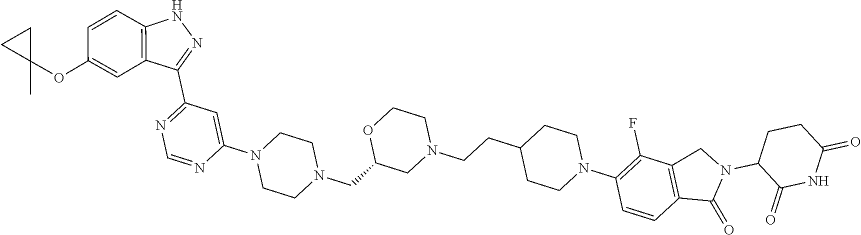 [SMILES: CC1(Oc2ccc3[nH]nc(-c4cc(N5CCN(C[C@H]6CN(CCC7CCN(c8ccc9c(c8F)CN(C8CCC(=O)NC8=O)C9=O)CC7)CCO6)CC5)ncn4)c3c2)CC1] |
| 18 | 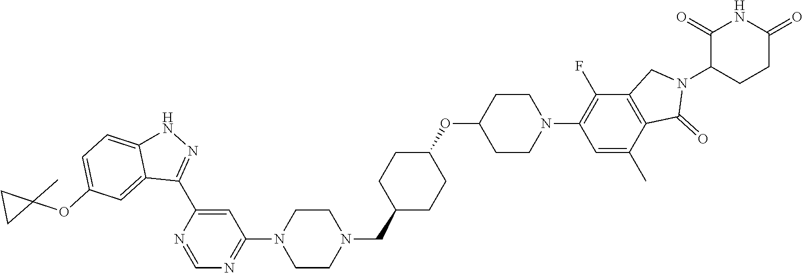 [SMILES: Cc1cc(N2CCC(O[C@H]3CC[C@H](CN4CCN(c5cc(-c6n[nH]c7ccc(OC8(C)CC8)cc67)ncn5)CC4)CC3)CC2)c(F)c2c1C(=O)N(C1CCC(=O)NC1=O)C2] |
| 19 | 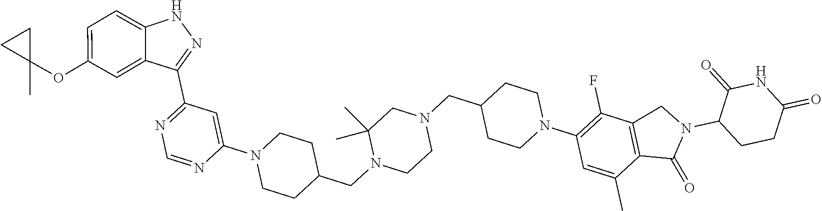 [SMILES: Cc1cc(N2CCC(CN3CCN(CC4CCN(c5cc(-c6n[nH]c7ccc(OC8(C)CC8)cc67)ncn5)CC4)C(C)(C)C3)CC2)c(F)c2c1C(=O)N(C1CCC(=O)NC1=O)C2] |
| 20 | 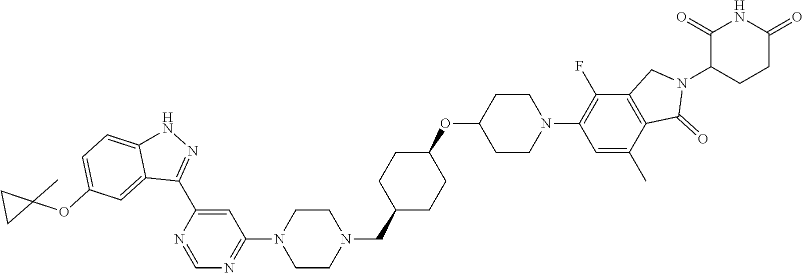 [SMILES: Cc1cc(N2CCC(O[C@H]3CC[C@@H](CN4CCN(c5cc(-c6n[nH]c7ccc(OC8(C)CC8)cc67)ncn5)CC4)CC3)CC2)c(F)c2c1C(=O)N(C1CCC(=O)NC1=O)C2] |
| 21 | 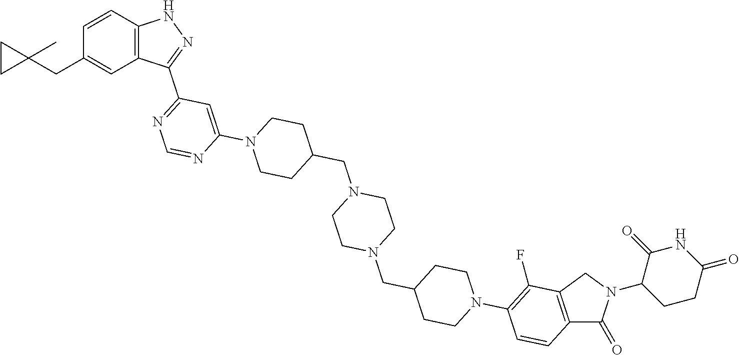 [SMILES: CC1(Cc2ccc3[nH]nc(-c4cc(N5CCC(CN6CCN(CC7CCN(c8ccc9c(c8F)CN(C8CCC(=O)NC8=O)C9=O)CC7)CC6)CC5)ncn4)c3c2)CC1] |
| 22 | 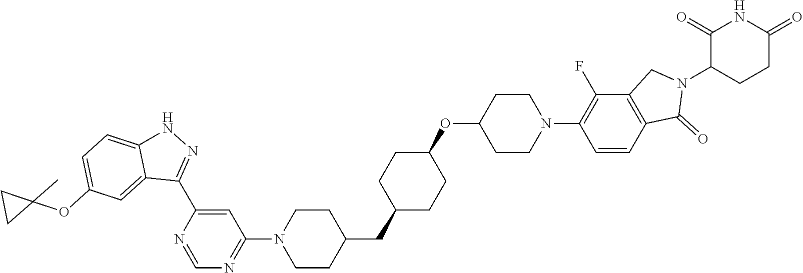 [SMILES: CC1(Oc2ccc3[nH]nc(-c4cc(N5CCC(C[C@H]6CC[C@@H](OC7CCN(c8ccc9c(c8F)CN(C8CCC(=O)NC8=O)C9=O)CC7)CC6)CC5)ncn4)c3c2)CC1] |
| 23 | 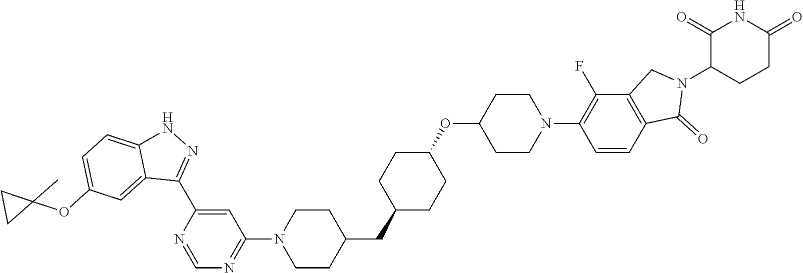 [SMILES: CC1(Oc2ccc3[nH]nc(-c4cc(N5CCC(C[C@H]6CC[C@H](OC7CCN(c8ccc9c(c8F)CN(C8CCC(=O)NC8=O)C9=O)CC7)CC6)CC5)ncn4)c3c2)CC1] |
| 24 | 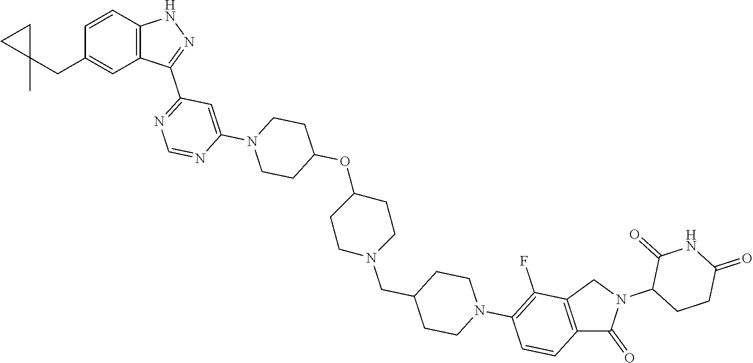 [SMILES: CC1(Cc2ccc3[nH]nc(-c4cc(N5CCC(OC6CCN(CC7CCN(c8ccc9c(c8F)CN(C8CCC(=O)NC8=O)C9=O)CC7)CC6)CC5)ncn4)c3c2)CC1] |
| 25 | 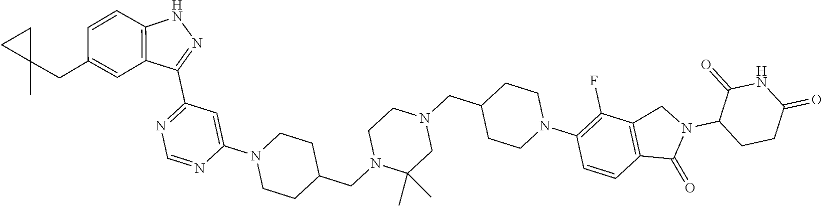 [SMILES: CC1(Cc2ccc3[nH]nc(-c4cc(N5CCC(CN6CCN(CC7CCN(c8ccc9c(c8F)CN(C8CCC(=O)NC8=O)C9=O)CC7)CC6(C)C)CC5)ncn4)c3c2)CC1] |
| 26 | 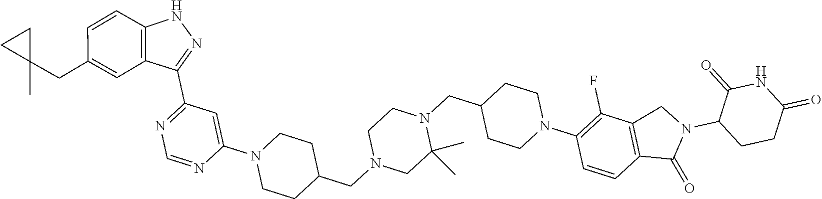 [SMILES: CC1(Cc2ccc3[nH]nc(-c4cc(N5CCC(CN6CCN(CC7CCN(c8ccc9c(c8F)CN(C8CCC(=O)NC8=O)C9=O)CC7)C(C)(C)C6)CC5)ncn4)c3c2)CC1] |
| 27 | 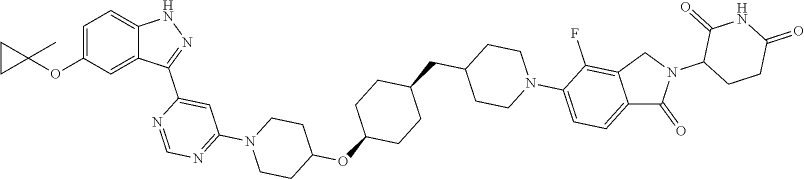 [SMILES: CC1(Oc2ccc3[nH]nc(-c4cc(N5CCC(O[C@H]6CC[C@@H](CC7CCN(c8ccc9c(c8F)CN(C8CCC(=O)NC8=O)C9=O)CC7)CC6)CC5)ncn4)c3c2)CC1] |
| 28 | 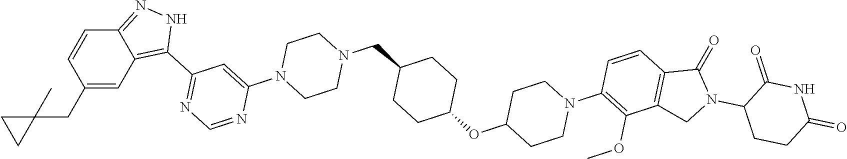 [SMILES: COc1c(N2CCC(O[C@H]3CC[C@H](CN4CCN(c5cc(-c6[nH]nc7ccc(CC8(C)CC8)cc67)ncn5)CC4)CC3)CC2)ccc2c1CN(C1CCC(=O)NC1=O)C2=O] |
| 29 | 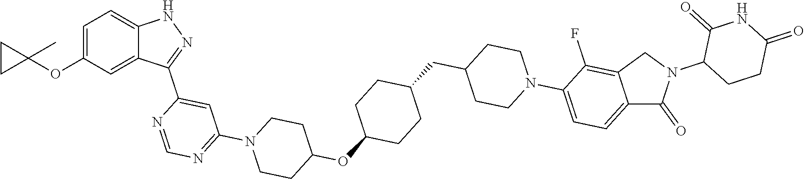 [SMILES: CC1(Oc2ccc3[nH]nc(-c4cc(N5CCC(O[C@H]6CC[C@H](CC7CCN(c8ccc9c(c8F)CN(C8CCC(=O)NC8=O)C9=O)CC7)CC6)CC5)ncn4)c3c2)CC1] |
| | and |
| 30 | 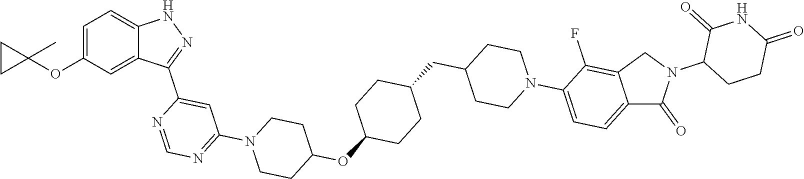 [SMILES: CC1(Oc2ccc3[nH]nc(-c4cc(N5CCC(O[C@H]6CC[C@H](CC7CCN(c8ccc9c(c8F)CN(C8CCC(=O)NC8=O)C9=O)CC7)CC6)CC5)ncn4)c3c2)CC1] |

or a pharmaceutically acceptable salt thereof.

In embodiments, the compound is selected from:

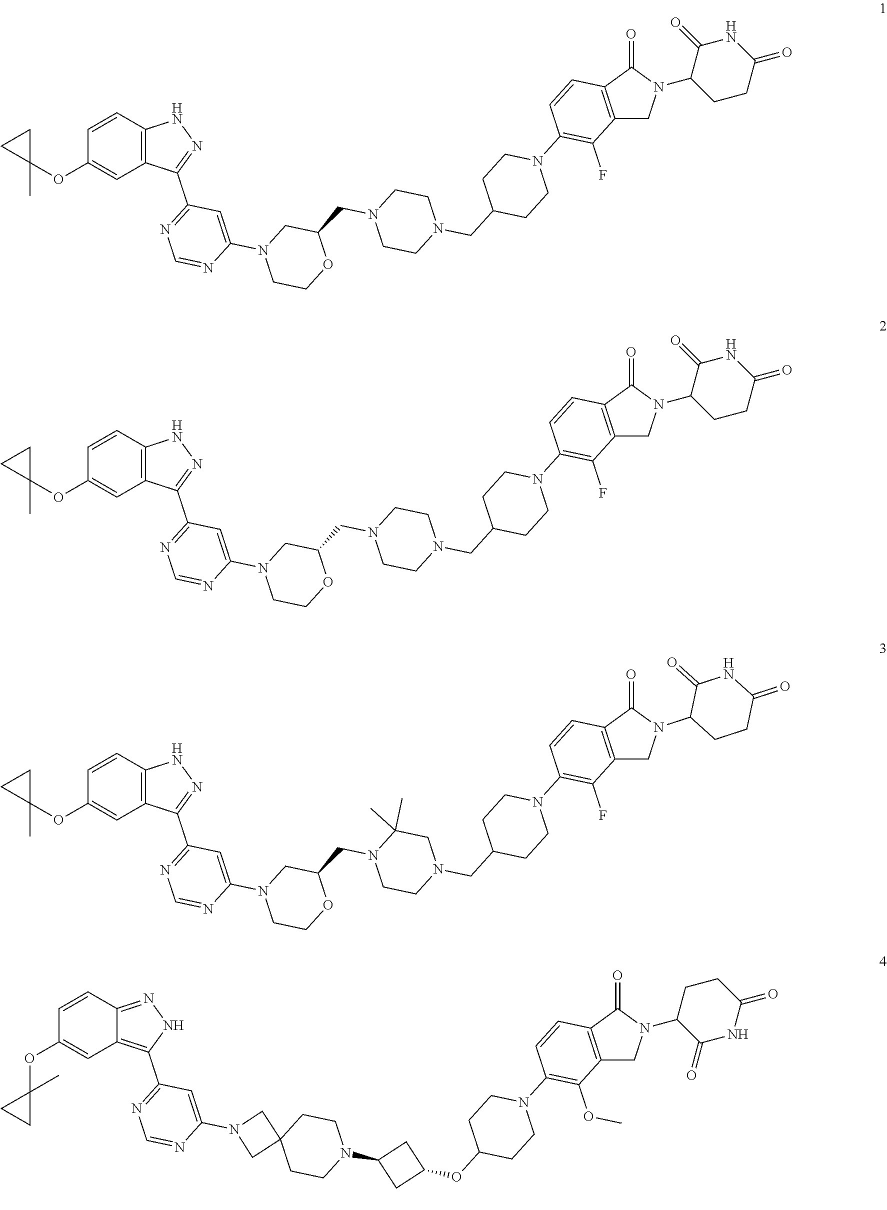 [SMILES: CC1(Oc2ccc3[nH]nc(-c4cc(N5CCO[C@H](CN6CCN(CC7CCN(c8ccc9c(c8F)CN(C8CCC(=O)NC8=O)C9=O)CC7)CC6)C5)ncn4)c3c2)CC1 ; CC1(Oc2ccc3[nH]nc(-c4cc(N5CCO[C@@H](CN6CCN(CC7CCN(c8ccc9c(c8F)CN(C8CCC(=O)NC8=O)C9=O)CC7)CC6)C5)ncn4)c3c2)CC1 ; CC1(Oc2ccc3[nH]nc(-c4cc(N5CCO[C@H](CN6CCN(CC7CCN(c8ccc9c(c8F)CN(C8CCC(=O)NC8=O)C9=O)CC7)CC6(C)C)C5)ncn4)c3c2)CC1 ; COc1c(N2CCC(O[C@H]3C[C@H](N4CCC5(CC4)CN(c4cc(-c6[nH]nc7ccc(OC8(C)CC8)cc67)ncn4)C5)C3)CC2)ccc2c1CN(C1CCC(=O)NC1=O)C2=O]

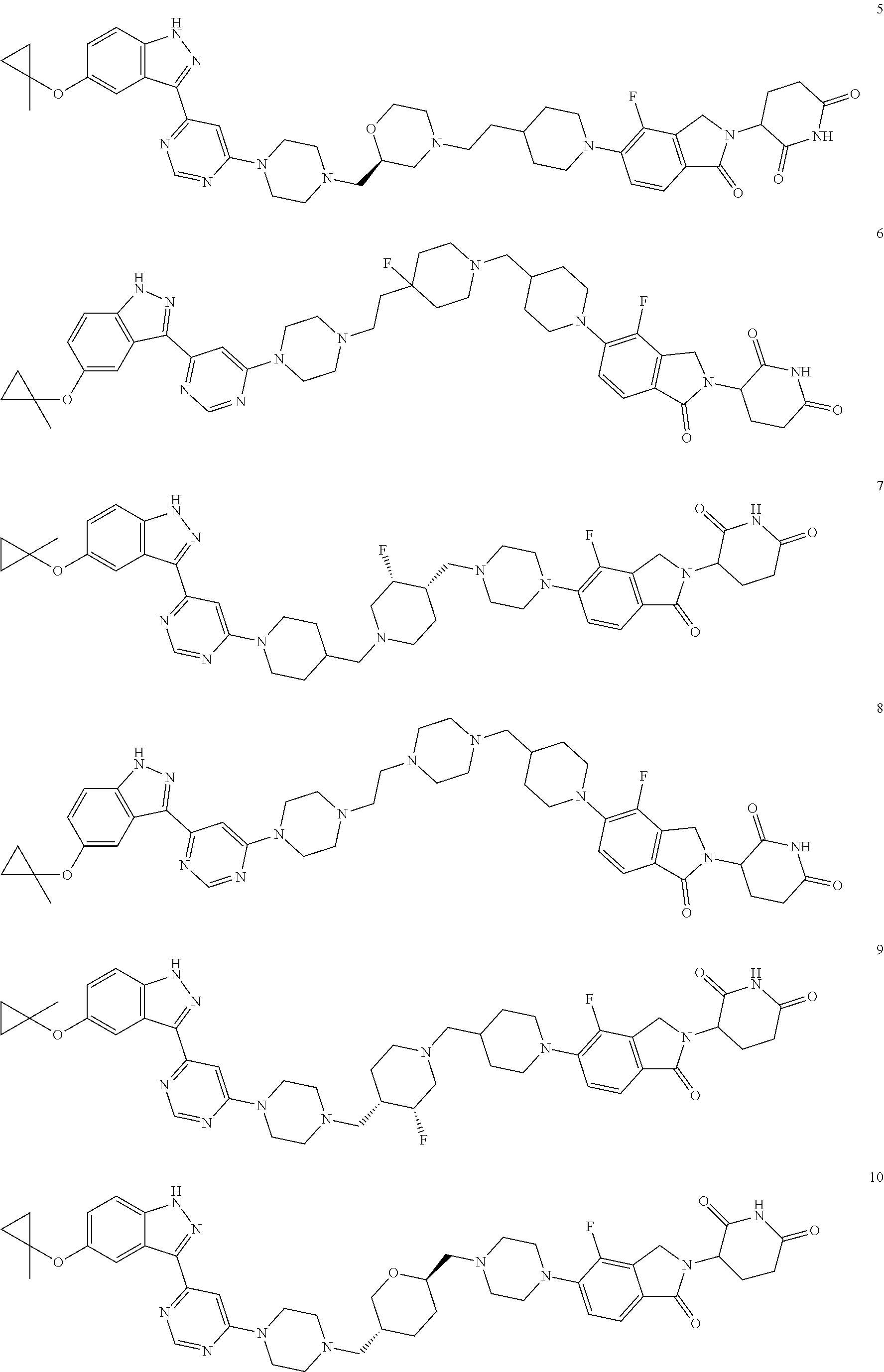

or a pharmaceutically acceptable salt thereof.

In embodiments, the compound is selected from:

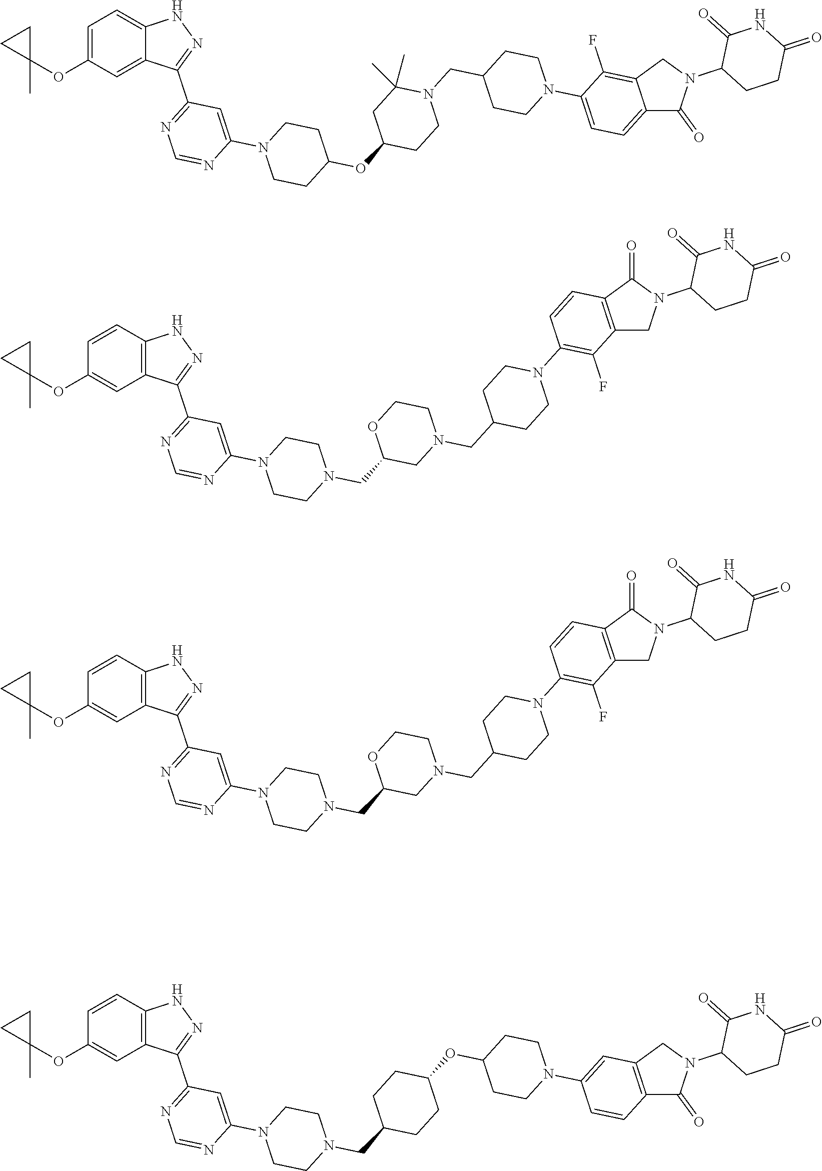 [SMILES: CC1(Oc2ccc3[nH]nc(-c4cc(N5CCC(O[C@@H]6CCN(CC7CCN(c8ccc9c(c8F)CN(C8CCC(=O)NC8=O)C9=O)CC7)C(C)(C)C6)CC5)ncn4)c3c2)CC1 ; CC1(Oc2ccc3[nH]nc(-c4cc(N5CCN(C[C@H]6CN(CC7CCN(c8ccc9c(c8F)CN(C8CCC(=O)NC8=O)C9=O)CC7)CCO6)CC5)ncn4)c3c2)CC1 ; CC1(Oc2ccc3[nH]nc(-c4cc(N5CCN(C[C@@H]6CN(CC7CCN(c8ccc9c(c8F)CN(C8CCC(=O)NC8=O)C9=O)CC7)CCO6)CC5)ncn4)c3c2)CC1 ; CC1(Oc2ccc3[nH]nc(-c4cc(N5CCN(C[C@H]6CC[C@H](OC7CCN(c8ccc9c(c8)CN(C8CCC(=O)NC8=O)C9=O)CC7)CC6)CC5)ncn4)c3c2)CC1]

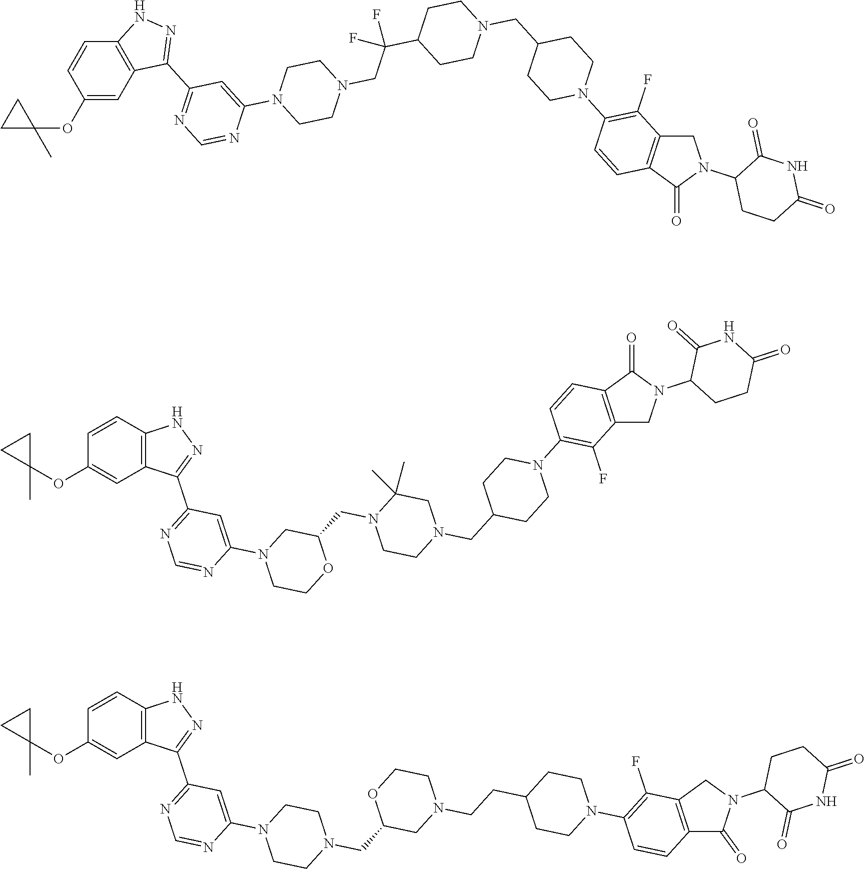 [SMILES: CC1(Oc2ccc3[nH]nc(-c4cc(N5CCN(CC(F)(F)C6CCN(CC7CCN(c8ccc9c(c8F)CN(C8CCC(=O)NC8=O)C9=O)CC7)CC6)CC5)ncn4)c3c2)CC1 ; CC1(Oc2ccc3[nH]nc(-c4cc(N5CCO[C@@H](CN6CCN(CC7CCN(c8ccc9c(c8F)CN(C8CCC(=O)NC8=O)C9=O)CC7)CC6(C)C)C5)ncn4)c3c2)CC1 ; CC1(Oc2ccc3[nH]nc(-c4cc(N5CCN(C[C@H]6CN(CCC7CCN(c8ccc9c(c8F)CN(C8CCC(=O)NC8=O)C9=O)CC7)CCO6)CC5)ncn4)c3c2)CC1]

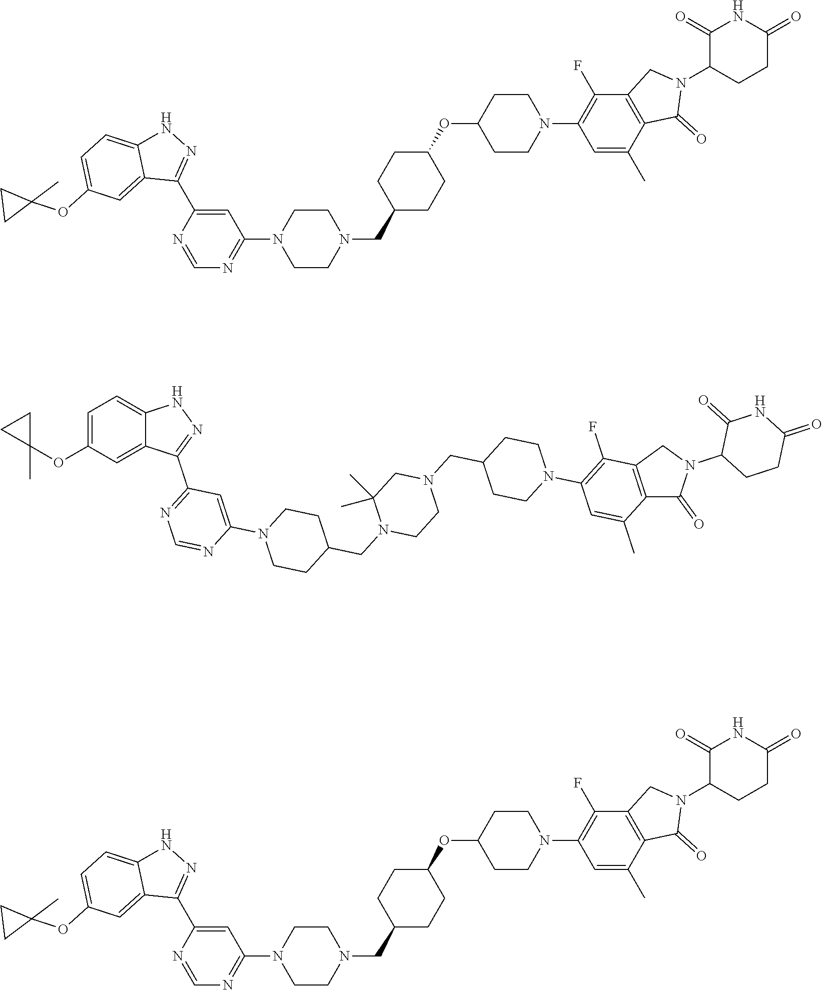 [SMILES: Cc1cc(N2CCC(O[C@H]3CC[C@H](CN4CCN(c5cc(-c6n[nH]c7ccc(OC8(C)CC8)cc67)ncn5)CC4)CC3)CC2)c(F)c2c1C(=O)N(C1CCC(=O)NC1=O)C2 ; Cc1cc(N2CCC(CN3CCN(CC4CCN(c5cc(-c6n[nH]c7ccc(OC8(C)CC8)cc67)ncn5)CC4)C(C)(C)C3)CC2)c(F)c2c1C(=O)N(C1CCC(=O)NC1=O)C2 ; Cc1cc(N2CCC(O[C@H]3CC[C@@H](CN4CCN(c5cc(-c6n[nH]c7ccc(OC8(C)CC8)cc67)ncn5)CC4)CC3)CC2)c(F)c2c1C(=O)N(C1CCC(=O)NC1=O)C2]

or a pharmaceutically acceptable salt thereof.

In embodiments, the compound is selected from:

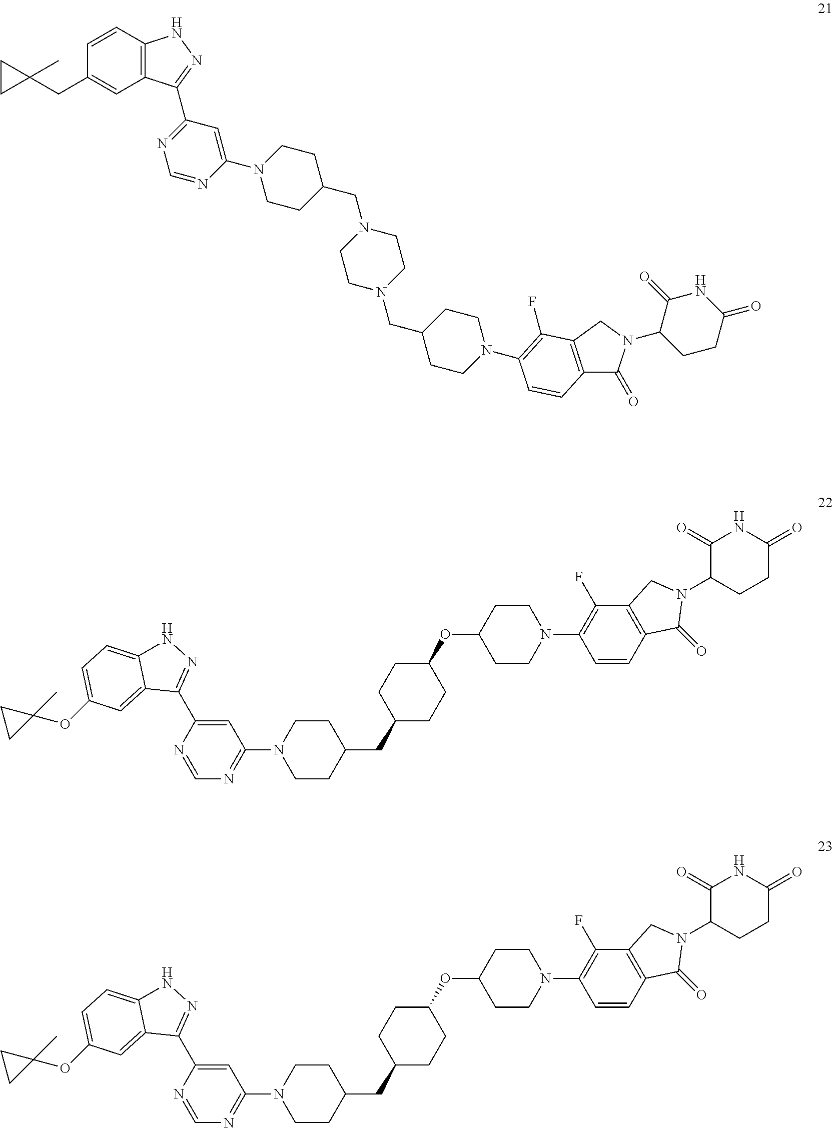 [SMILES: CC1(Cc2ccc3[nH]nc(-c4cc(N5CCC(CN6CCN(CC7CCN(c8ccc9c(c8F)CN(C8CCC(=O)NC8=O)C9=O)CC7)CC6)CC5)ncn4)c3c2)CC1 ; CC1(Oc2ccc3[nH]nc(-c4cc(N5CCC(C[C@H]6CC[C@@H](OC7CCN(c8ccc9c(c8F)CN(C8CCC(=O)NC8=O)C9=O)CC7)CC6)CC5)ncn4)c3c2)CC1 ; CC1(Oc2ccc3[nH]nc(-c4cc(N5CCC(C[C@H]6CC[C@H](OC7CCN(c8ccc9c(c8F)CN(C8CCC(=O)NC8=O)C9=O)CC7)CC6)CC5)ncn4)c3c2)CC1]

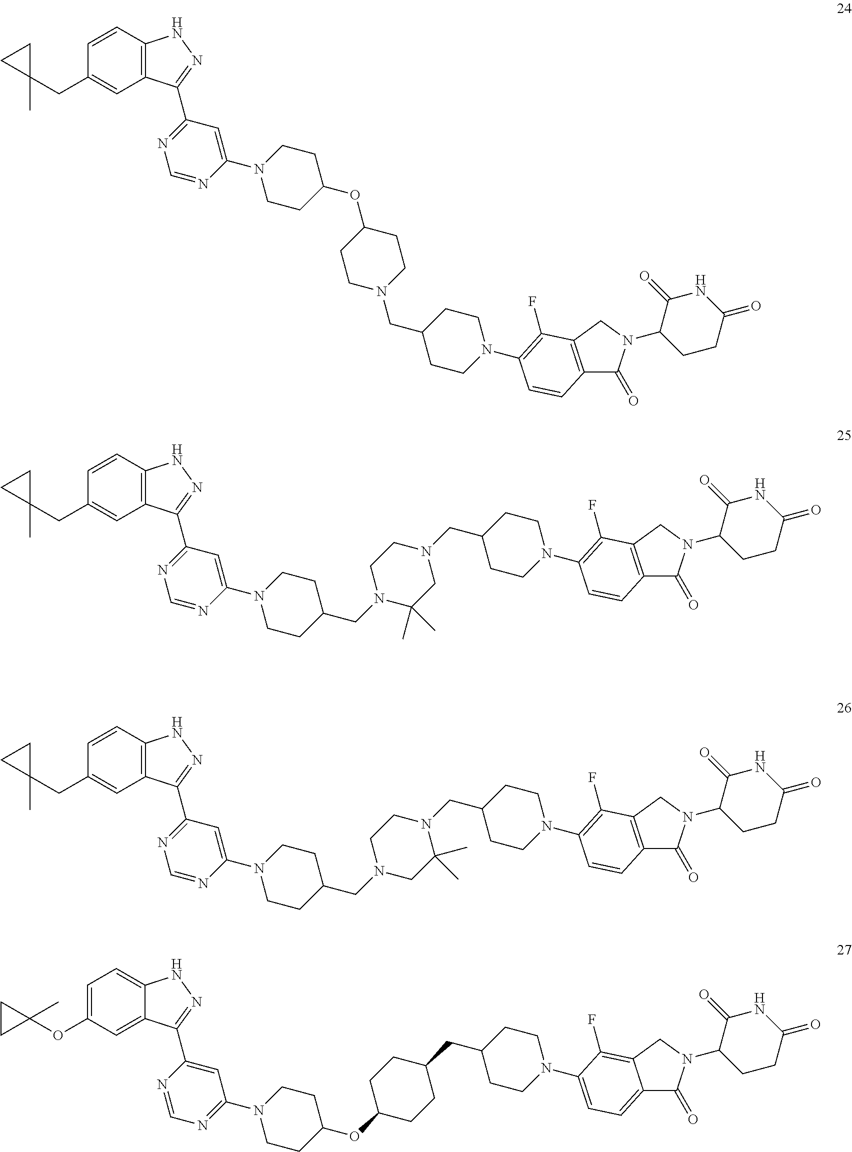 [SMILES: CC1(Cc2ccc3[nH]nc(-c4cc(N5CCC(OC6CCN(CC7CCN(c8ccc9c(c8F)CN(C8CCC(=O)NC8=O)C9=O)CC7)CC6)CC5)ncn4)c3c2)CC1 ; CC1(Cc2ccc3[nH]nc(-c4cc(N5CCC(CN6CCN(CC7CCN(c8ccc9c(c8F)CN(C8CCC(=O)NC8=O)C9=O)CC7)CC6(C)C)CC5)ncn4)c3c2)CC1 ; CC1(Cc2ccc3[nH]nc(-c4cc(N5CCC(CN6CCN(CC7CCN(c8ccc9c(c8F)CN(C8CCC(=O)NC8=O)C9=O)CC7)C(C)(C)C6)CC5)ncn4)c3c2)CC1 ; CC1(Oc2ccc3[nH]nc(-c4cc(N5CCC(O[C@H]6CC[C@@H](CC7CCN(c8ccc9c(c8F)CN(C8CCC(=O)NC8=O)C9=O)CC7)CC6)CC5)ncn4)c3c2)CC1]

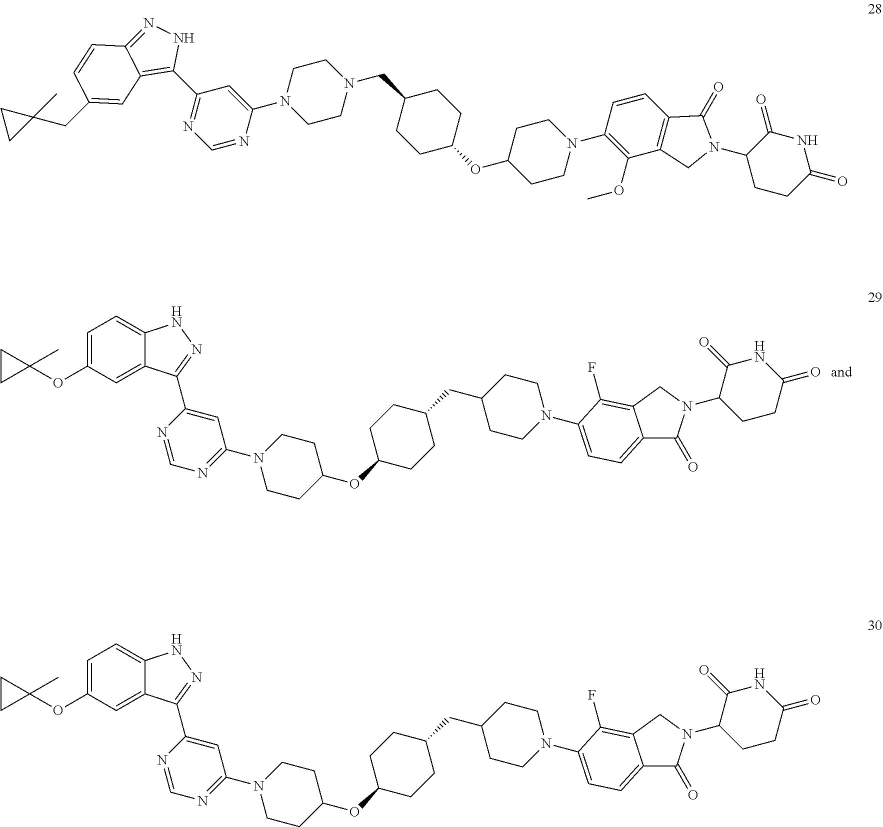 [SMILES: COc1c(N2CCC(O[C@H]3CC[C@H](CN4CCN(c5cc(-c6[nH]nc7ccc(CC8(C)CC8)cc67)ncn5)CC4)CC3)CC2)ccc2c1CN(C1CCC(=O)NC1=O)C2=O ; CC1(Oc2ccc3[nH]nc(-c4cc(N5CCC(O[C@H]6CC[C@H](CC7CCN(c8ccc9c(c8F)CN(C8CCC(=O)NC8=O)C9=O)CC7)CC6)CC5)ncn4)c3c2)CC1 ; "and" ; CC1(Oc2ccc3[nH]nc(-c4cc(N5CCC(O[C@H]6CC[C@H](CC7CCN(c8ccc9c(c8F)CN(C8CCC(=O)NC8=O)C9=O)CC7)CC6)CC5)ncn4)c3c2)CC1]

or a pharmaceutically acceptable salt thereof.

These compounds are also referred to herein as “the compounds described herein” or “the compounds disclosed herein,” etc.

The compounds disclosed herein may exist as tautomers and optical isomers (e.g., enantiomers, diastereomers, diastereomeric mixtures, racemic mixtures, and the like).

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds disclosed herein can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In the compounds provided herein, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Also, unless otherwise stated, when a position is designated specifically as “D” or “deuterium”, the position is understood to have deuterium at an abundance that is at least 3000 times greater than the natural abundance of deuterium, which is 0.015% (i.e., at least 45% incorporation of deuterium).

In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Pharmaceutical Compositions

Provided herein are pharmaceutical compositions comprising a compound described herein, or a pharmaceutically acceptable salt thereof, and a pharmaceutically acceptable carrier.

In embodiments, the pharmaceutical composition further comprises an additional bioactive agent. In embodiments, the additional bioactive agent is an anti-inflammatory, a chemotherapy agent, or an immunomodulatory agent.

The pharmaceutical compositions effect targeted protein degradation in a patient or subject, for example, an animal such as a human, and can be used for treating or ameliorating disease states or conditions that are modulated by degrading the target protein. In embodiments, the therapeutic compositions as described herein may be used to effectuate the degradation of protein for the treatment or amelioration of LRRK2-mediated inflammatory diseases, autoimmune diseases, or cancer. In embodiments, the disease is idiopathic PD, LRRK2 mutation-associated PD (e.g., PD associated with one or more LRRK2 activating mutations), primary tauopathies (e.g., supranuclear palsy (PSP) or corticobasal degeneration (CBD)), lewy body dementia, Crohn's Disease, Leprosy (e.g., Leprosy with type 1 inflammatory reactions), systemic lupus erythematosus (SLE), and/or neuroinflammation.

### Methods of Treatment

Provided herein are methods of treating a disease, a disorder, or a symptom causally related to LRRK2 comprising administering to a subject an effective amount of a compound provided herein.

In embodiments, the disease or disorder is Parkinson's disease (PD), LRRK2 mutation associated PD, primary tauopathies, lewy body dementia, Crohn's Disease, Leprosy, neuroinflammation, Progressive Supranuclear Palsy, Picks disease, FTDtau, TDP-43 Frontal Temporal Dementia, TDP-43 ALS, c9orf ALS, Huntington's disease, spinocerebellar ataxias (SCAs) 1, 2, 3, 6, 7 and 17, dentatorubral pallidoluysian atrophy (DRPLA), systemic lupus erythematosus (SLE), or Kennedy's disease. In embodiments, the disease is PD. In embodiments, the disease is LRRK2 mutation associated PD. In embodiments, the disease is lewy body dementia.

In embodiments, the disease or disorder is Parkinson's disease (PD), LRRK2 mutation associated PD, primary tauopathies, lewy body dementia, Crohn's Disease, Leprosy, neuroinflammation, Progressive Supranuclear Palsy, Picks disease, FTDtau, TDP-43 Frontal Temporal Dementia, TDP-43 ALS, c9orf ALS, Huntington's disease, spinocerebellar ataxias (SCAs) 1, 2, 3, 6, 7 and 17, dentatorubral pallidoluysian atrophy (DRPLA), or Kennedy's disease. In embodiments, the disease is PD. In embodiments, the disease is LRRK2 mutation associated PD. In embodiments, the disease is lewy body dementia.

Provided herein are methods of treating Parkinson's disease comprising administering to a subject an effective amount of a compound of the present disclosure.

In embodiments, the Parkinson's disease is LRRK2 mutation associated Parkinson's disease.

The present disclosure also relates to methods for treating a disease state or ameliorating one or more symptoms of a disease or condition in a subject in need thereof by degrading the LRRK2 protein (e.g., a wildtype LRRK2 protein or an LRRK2 mutant protein (e.g., a LRRK2 mutant protein including one or more mutation selected from G2019S, I2020T, N1437H, R1441G/C/H, and Y1699C) comprising administering to said patient or subject an effective amount of at least one compound as described herein, optionally in combination with a pharmaceutically acceptable carrier, additive or excipient, and optionally coadministered with an additional bioactive agent, wherein the composition is effective for treating or ameliorating the disease or disorder or one or more symptoms thereof in the subject.

The method according to the present disclosure may be used to treat certain disease states, conditions or symptoms including inflammatory disease, autoimmune disease, or cancer, by virtue of the administration of effective amounts of at least one compound described herein. For example, the method according to the present disclosure may be used to reat one or more of Parkinson's Disease (PD), idiopathic PD, LRRK2 mutation associated PD (e.g., PD associated with one or more LRRK2 activating mutations), primary tauopathies (e.g., supranuclear palsy (PSP) or corticobasal degeneration (CBD)), lewy body dementia, Crohn's Disease, Leprosy (e.g., Leprosy with type 1 inflammatory reactions), systemic lupus erythematosus (SLE), and neuroinflammation (such as is observed in Alzheimer's disease, PD, multiple sclerosis, traumatic brain injury, spinal cord injury, etc.). In another example, the method according to the present disclosure may be used to reat one or more of Parkinson's Disease (PD), idiopathic PD, LRRK2 mutation associated PD (e.g., PD associated with one or more LRRK2 activating mutations), primary tauopathies (e.g., supranuclear palsy (PSP) or corticobasal degeneration (CBD)), lewy body dementia, Crohn's Disease, Leprosy (e.g., Leprosy with type 1 inflammatory reactions), and neuroinflammation (such as is observed in Alzheimer's disease, PD, multiple sclerosis, traumatic brain injury, spinal cord injury, etc.).

The description also provides therapeutic methods comprising administration of an effective amount of a compound as described herein or salt form thereof, and a pharmaceutically acceptable carrier. The therapeutic methods are useful to effect protein degradation in a patient or subject, for example, an animal such as a human, for treating or ameliorating a disease state, condition or related symptom that may be treated through targeted protein degradation.

The description provides therapeutic methods for effectuating the degradation of proteins of interest for the treatment or amelioration of a disease, e.g., Parkinson's Disease (PD), primary tauopathies, lewy body dementia, Crohn's Disease, systemic lupus erythematosus (SLE), Leprosy, and/or neuroinflammation. In embodiments, the disease is idiopathic PD, LRRK2 mutation associated PD (e.g., PD associated with one or more LRRK2 activating mutations), PSP, CBD, Leprosy with type 1 inflammatory reactions, Alzheimer's disease, PD, multiple sclerosis, traumatic brain injury, systemic lupus erythematosus (SLE), and/or spinal cord injury. The description provides methods of ubiquitinating/degrading a target protein in a cell. In embodiments, the method comprises administering a compound disclosed herein. The control or reduction of specific protein levels in cells of a subject as afforded by the present disclosure provides treatment of a disease state, condition, or symptom. In embodiments, the methods comprise administering an effective amount of a compound as described herein, optionally including a pharmaceutically acceptable excipient, carrier, adjuvant, another bioactive agent, or combination thereof.

The description provides therapeutic methods for effectuating the degradation of proteins of interest for the treatment or amelioration of a disease, e.g., Parkinson's Disease (PD), primary tauopathies, lewy body dementia, Crohn's Disease, Leprosy, and/or neuroinflammation. In embodiments, the disease is idiopathic PD, LRRK2 mutation associated PD (e.g., PD associated with one or more LRRK2 activating mutations), PSP, CBD, Leprosy with type 1 inflammatory reactions, Alzheimer's disease, PD, multiple sclerosis, traumatic brain injury, and/or spinal cord injury. The description provides methods of ubiquitinating/degrading a target protein in a cell. In embodiments, the method comprises administering a compound disclosed herein. The control or reduction of specific protein levels in cells of a subject as afforded by the present disclosure provides treatment of a disease state, condition, or symptom. In embodiments, the methods comprise administering an effective amount of a compound as described herein, optionally including a pharmaceutically acceptable excipient, carrier, adjuvant, another bioactive agent, or combination thereof.

Provided herein are a process for making a small molecule that can cause degradation of LRRK2 in a cell, comprising the steps of: (i) providing a small molecule that binds to the LRRK2 or a mutated form thereof; (ii) providing an E3 ubiquitin ligase binding moiety (ULM), preferably a CLM such as thalidomide, pomalidomide, lenalidomide or an analog thereof; and (iii) covalently coupling the small molecule of step (i) to the ULM of step (ii) via a chemical linking group (L) to form a compound which binds to both a cereblon E3 ubiquitin ligase and LRRK2 protein and/or mutated form in the cell, such that the cereblon E3 ubiquitin ligase is in proximity to, and ubiquitinates the LRRK2 protein bound thereto, such that the ubiquitinated LRRK2 protein is then degraded.

Provided herein are methods for detecting whether a small molecule can trigger degradation of a LRRK2 protein in a cell, the method comprising the steps of: (i) providing a small molecule for which the ability to trigger degradation of LRRK2 protein in a cell is to be detected, said small molecule comprising the structure: CLM-L-PTM, wherein CLM is a cereblon E3 ubiquitin ligase binding moiety capable of binding a cereblon E3 ubiquitin ligase in a cell, which CLM is thalidomide, pomalidomide, lenalidomide, or an analog thereof; PTM is a protein targeting moiety, which is a small molecule that binds to LRRK2 and/or mutated LRRK2 form thereof, said LRRK2 having at least one lysine residue available to be ubiquitinated by a cereblon E3 ubiquitin ligase bound to the CLM of the molecule; and L is a chemical linking group that covalently links the CLM to the PTM to form the small molecule; (ii) incubating a LRRK2 protein-expressing cell in the presence of the small molecule of step (i); and (iii) detecting whether the LRRK2 protein in the cell has been degraded.

In embodiments, the small molecule capable of binding LRRK2, is a small molecule that binds LRRK2. In embodiments, the small molecule that binds the LRRK2 protein is as described herein.

Provided herein are methods of treating a human patient in need of said treatment of a disease state, condition, or symptom causally related to LRRK2, and/or LRRK2 mutated form, expression, over-expression, mutation, aggregation, accumulation, misfolding or dysregulation where the degradation of the LRRK2 protein will produce a therapeutic effect in the patient, the method comprising administering to the patient an effective amount of a compound according to the present disclosure, optionally in combination with another bioactive agent.

Provided herein are methods of treating a human patient in need of said treatment of a disease state, condition, or symptom causally related to alpha-synuclein expression, over-expression, mutation, aggregation, accumulation, misfolding or dysregulation where the degradation of the LRRK2 protein and/or mutated form thereof will produce a therapeutic effect in the patient, the method comprising administering to the patient an effective amount of a compound according to the present disclosure, optionally in combination with another bioactive agent.

Provided herein are methods of treating a human patient in need of said treatment of a disease state, condition, or symptom causally related to alpha-synuclein expression, over-expression, mutation, aggregation, misfolding or dysregulation where the degradation of the LRRK2 protein and/or mutated form thereof will produce a therapeutic effect in the patient, the method comprising administering to the patient an effective amount of a compound according to the present disclosure, optionally in combination with another bioactive agent.

Provided herein are methods of treating a human patient in need of said treatment of a disease state, condition, or symptom causally related to Tau expression, over-expression, mutation, aggregation, misfolding or dysregulation where the degradation of the LRRK2 protein and/or mutated form thereof will produce a therapeutic effect in the patient, the method comprising administering to the patient an effective amount of a compound according to the present disclosure, optionally in combination with another bioactive agent.

In embodiments, the disease state, condition, or symptom may be caused by a microbial agent or other exogenous agent such as a virus, bacteria, fungus, protozoa, or other microbe, or may be a disease state, which is caused by expression, overexpression, mutation, misfolding, or dysregulation of the protein, which leads to a disease state, condition, or symptom.

In embodiments, the disease state, condition, or symptom which may be treated using compounds according to the present disclosure include, for example, Parkinson's Disease (PD), idiopathic PD, LRRK2 mutation associated PD (e.g., PD associated with one or more LRRK2 activating mutations), primary tauopathies (e.g., supranuclear palsy (PSP) or corticobasal degeneration (CBD)), lewy body dementia, Crohn's Disease, Leprosy (e.g., Leprosy with type 1 inflammatory reactions), systemic lupus erythematosus (SLE), and/or neuroinflammation (such as is observed in Alzheimer's disease, PD, multiple sclerosis, traumatic brain injury, spinal cord injury, etc.).

In embodiments, the disease state, condition, or symptom which may be treated using compounds according to the present disclosure include, for example, Parkinson's Disease (PD), idiopathic PD, LRRK2 mutation associated PD (e.g., PD associated with one or more LRRK2 activating mutations), primary tauopathies (e.g., supranuclear palsy (PSP) or corticobasal degeneration (CBD)), lewy body dementia, Crohn's Disease, Leprosy (e.g., Leprosy with type 1 inflammatory reactions), and/or neuroinflammation (such as is observed in Alzheimer's disease, PD, multiple sclerosis, traumatic brain injury, spinal cord injury, etc.).

Provided herein are methods of treating or ameliorating at least one symptom of a disease or condition in a subject, comprising the steps of:
a) providing a subject identified as having a symptom of a disease or condition causally related to expression, overexpression, mutation, misfolding, or dysregulation of the LRRK2 protein and/or mutated form thereof in the subject, and the symptom of the disease or condition is treated or ameliorated by degrading the LRRK2 protein and/or mutated form thereof in cells of the subject; and
b) administering to the subject an effective amount of a compound comprising a small molecule of the present disclosure such that the LRRK2 protein and/or mutated form thereof is degraded, thereby treating, or ameliorating at least one symptom of a disease or condition in the subject.

### Administration/Dosages/Formulations

Liquid dosage forms for oral administration include pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

To prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants, or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams, and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compounds of the present disclosure can be administered intratympanically, wherein a long, narrow, bore needle is passed through the ear canal and through the eardrum to administer medications into the middle ear space where they are absorbed by the inner ear.

According to the methods of treatment of the present disclosure, disorders are treated or prevented in a subject, such as a human or other animal, by administering to the subject an effective amount of a compound of the disclosure, in such amounts and for such time as is necessary to achieve the desired result.

In general, compounds of the disclosure will be administered in effective amounts via any of the usual and acceptable modes known in the art, either singly or in combination with one or more therapeutic agents. An effective amount may vary widely depending on the severity of the disease, the age and relative health of the subject, the potency of the compound used and other factors. In general, satisfactory results are indicated to be obtained systemically at daily dosages from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, the amount or dose of the compounds of the present disclosure may range from about 0.1 mg/Kg to about 500 mg/Kg, alternatively from about 1 to about 50 mg/Kg. In general, treatment regimens according to the present disclosure comprise administration to a patient in need of such treatment from about 10 mg to about 1000 mg of the compound(s) of this disclosure per day in single or multiple doses. The amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present disclosure.

### Kits

Provided herein are kits comprising a compound capable of causing the degradation of LRRK2 in a subject in need thereof comprising one or more compounds disclosed herein, or pharmaceutically acceptable salts thereof, and instructions for use in treating a disorder associated with LRRK2.

Provided herein are kits comprising a compound disclosed herein, or a pharmaceutically acceptable salt thereof for the treatment of any of the indications disclosed herein.

Those skilled in the art will recognize or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### Examples

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### General Schemes:

Indazole-based PTMs were made in the following generic manner shown in General Scheme 1. Nucleophilic aromatic substitution (SnAr) of appropriately substituted fluoronitrobenzenes with metal alkoxides or alkylation of substituted phenolic nitrobenzenes provided aromatic ethers which were subsequently converted to methyl derivatives by transition metal catalyzed coupling with trimethylboroxine. Nitro reduction followed by acetylation and treatment with alkyl nitrite in the presence of KOAc and Ac2O afforded the appropriately substituted indazole intermediates. The indazoles could then be protected at either N1 or N2 and subjected to C3 metalation followed by transition metal mediated coupling to provide the appropriately substituted 1-protected-3-(6-chloropyrimidin-4-yl)-1H-indazoles or substituted 2-protected-3-(6-chloropyrimidyl-4-yl)-2H-indazoles.

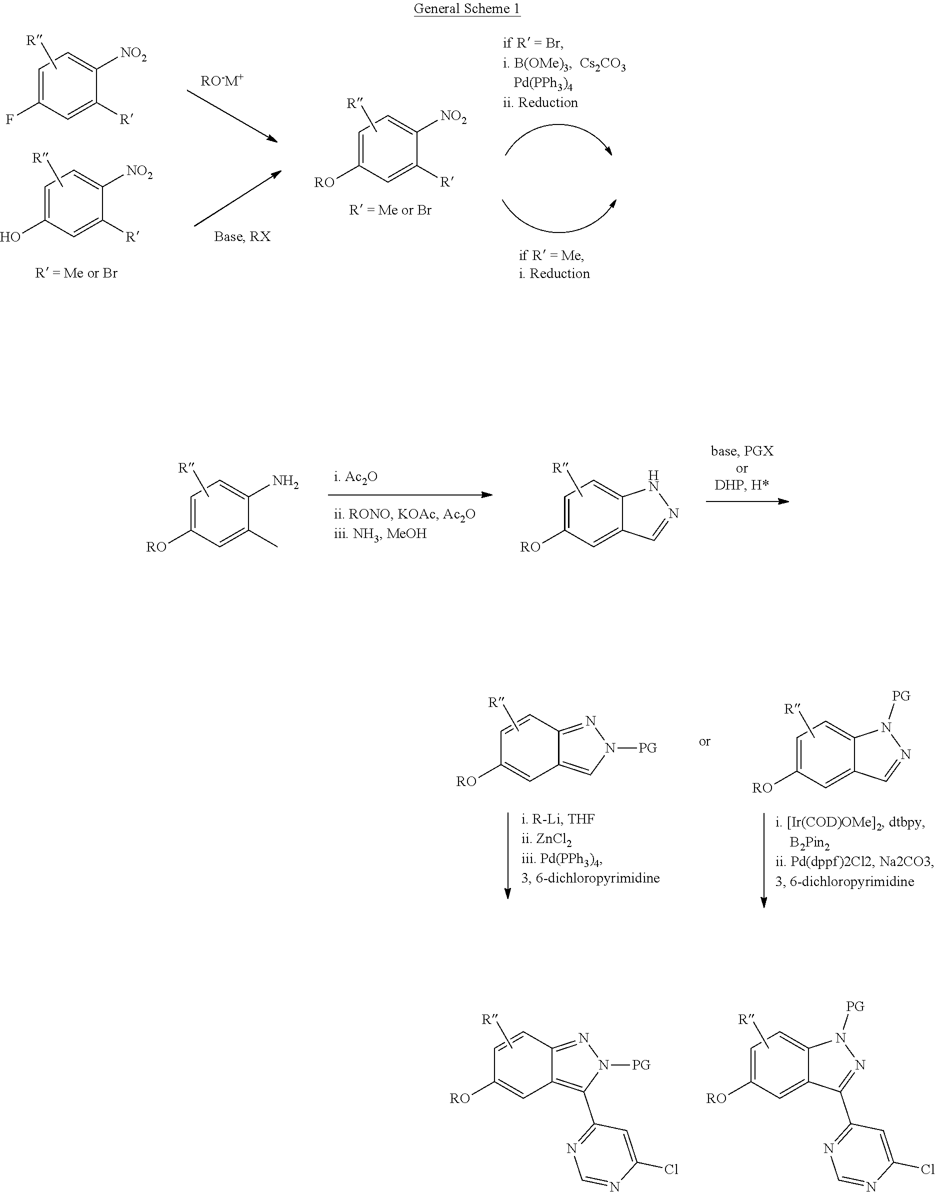

The substituted-protected-indazoles could then be elaborated as shown in General Scheme 2. Protecting group removal followed by SnAr with an appropriately elaborated amine can directly afford the final compound or the steps can be reversed with deprotection following the SnAr reaction to deliver the final compound.

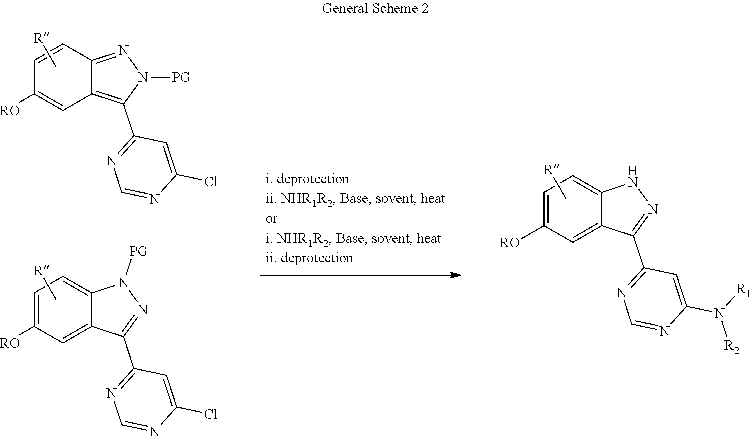

Alternatively, the amines used in the SnAr reaction can be intermediates with a reactive functional group (Scheme 3) which allows formation of the final compound by a subsequent coupling reaction. In other cases, a protected functional group on the amine maybe required which will need subsequent deprotection prior to coupling.

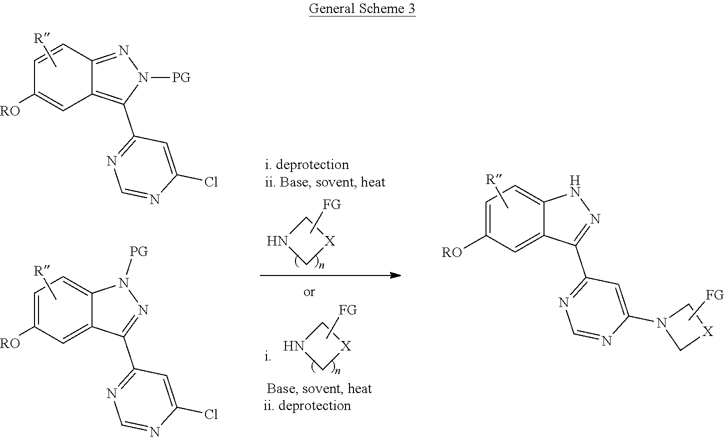

Generic syntheses of the ubiquitin ligase moiety (ULM) is shown in General Scheme 4. Nucleophilic displacement (SnAr) of an appropriately substituted arylfluoride or transition metal mediated coupling of an aryl bromide with the appropriately substituted amine provided the desired ULM intermediates or target compounds.

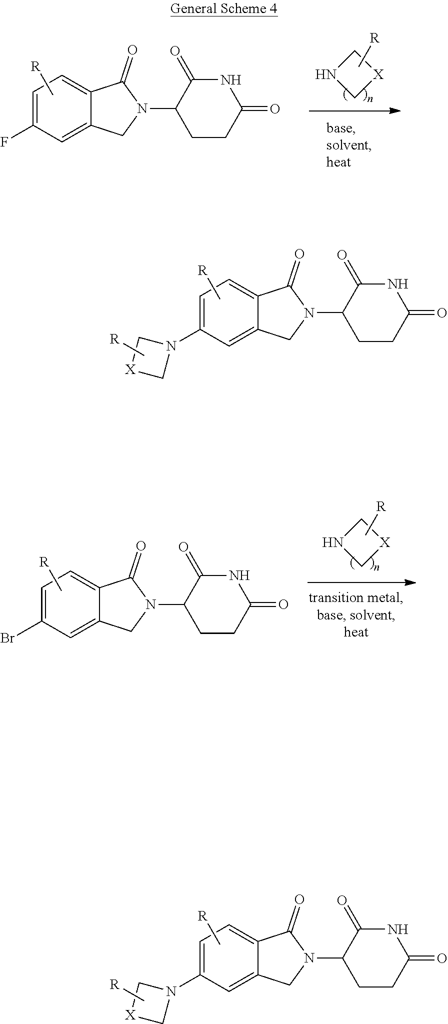

### Exemplary Synthesis of Intermediate 1: 5-(1-methylcyclopropoxy)-3-(6-piperazin-1-ylpyrimidin-4-yl)-1H-indazole

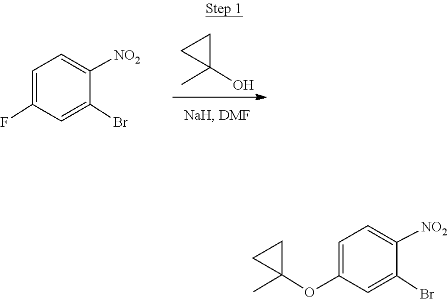

To a solution of 2-bromo-4-fluoro-1-nitro-benzene (16.78 g, 76.28 mmol, 1.1 eq) and 1-methylcyclopropanol (5 g, 69.34 mmol, 1 eq) in DMF (160 mL) was added NaH (4.16 g, 104.01 mmol, 60% in mineral oil, 1.5 eq) in one portion at 0° C. under N₂. Then the mixture was heated to 20° C. and stirred for 4 hours. TLC showed there were new spots. The residue was poured into water (200 mL) and stirred for 10 min. The aqueous phase was extracted with ethyl acetate (3×300 mL). The combined organic phase was washed with brine (2×200 mL), dried with anhydrous Na₂SO₄, filtered and concentrated in vacuum. The residue was purified by silica gel column chromatography (100-200 mesh silica gel, 0-2% of Ethyl acetate in Petroleum ether) to afford 2-bromo-4-(1-methylcyclopropoxy)-1-nitro-benzene (14.3 g, 52.56 mmol, 75.79% yield) as a yellow oil.

### Step 2

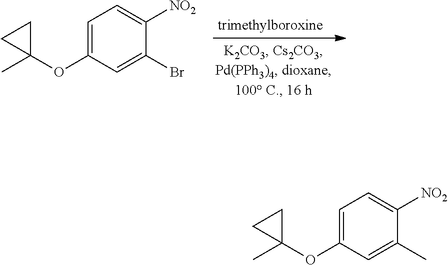

To a mixture of 2-bromo-4-(1-methylcyclopropoxy)-1-nitro-benzene (14.3 g, 52.56 mmol, 1 eq), K₂CO₃ (14.53 g, 105.11 mmol, 2 eq) and Cs₂CO₃ (17.12 g, 52.56 mmol, 1 eq) in 1,4-dioxane (100 mL) was added 2,4,6-trimethyl-1,3,5,2,4,6-trioxatriborinane (32.99 g, 131.39 mmol, 36.73 mL, 50% purity in EtOAc, 2.5 eq) and Pd(PPh₃)₄ (6.07 g, 5.26 mmol, 0.1 eq)) at 20° C., then heated to 100° C. and stirred for 16 hours to give yellow solution. TLC showed the reaction was completed. The reaction was cooled to 20° C. and concentrated under vacuum. To this residue was added PE:EtOAc (10:1, 100 mL), and the mixture was filtered through a pad of silica. The filter pad was washed with petroleum ether:EtOAc (10:1, 1000 mL) solvent. The residue was purified by silica gel chromatography (100-200 mesh silica gel, 0-1% of Ethyl acetate in Petroleum ether) to afford 2-methyl-4-(1-methylcyclopropoxy)-1-nitro-benzene (11 g, crude) as a yellow oil.

### Step 3

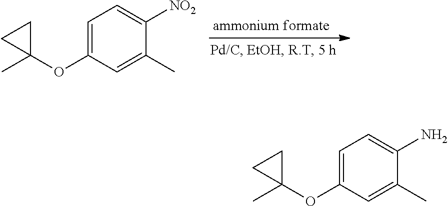

To a mixture of 2-methyl-4-(1-methylcyclopropoxy)-1-nitro-benzene (11 g, 53.08 mmol, 1 eq) in EtOH (100 mL) was added 10% of Pd/C (4 g, 5.31 mmol, 0.1 eq) and ammonium formate (40.17 g, 636.99 mmol, 12 eq) in one portion at 20° C. under N₂. The mixture was stirred at 20° C. for 2 h to give a black mixture. TLC showed the reaction was completed. The mixture was filtered through a pad of silica gel, washed with EtOAc (3×200 mL) and concentrated in vacuum. The residue was purified by silica gel chromatography (0-10% of Ethyl acetate in Petroleum ether) to afford 2-methyl-4-(1-methylcyclopropoxy) aniline (9.8 g, crude) as a red oil.

### Step 4

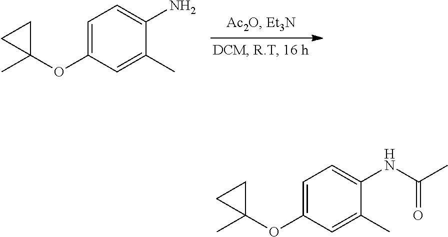

To a mixture of 2-methyl-4-(1-methylcyclopropoxy) aniline (9.8 g, 55.29 mmol, 1 eq) and Et₃N (13.99 g, 138.23 mmol, 19.24 mL, 2.5 eq) in DCM (100 mL) was added Ac₂O (11.29 g, 110.58 mmol, 10.36 mL, 2 eq) in one portion at 0° C. under N₂. The mixture was stirred at 0° C. for 30 min, then heated to 20° C. and stirred for 16 hours. TLC showed the reaction was completed. The reaction was quenched with a saturated solution of aqueous NaHCO₃ (30 mL) to adjusted pH=7-8 and extracted with DCM (3×50 mL). The combined organic phase was washed with brine (3×50 mL), dried with anhydrous Na₂SO₄, filtered and concentrated in vacuum. The residue was purified by silica gel chromatography (20-40% Ethyl acetate in Petroleum ether) to afford N-[2-methyl-4-(1-methylcyclopropoxy)phenyl]acetamide (9.3 g, 42.41 mmol, 76.71% yield) as a yellow oil.

### Step 5

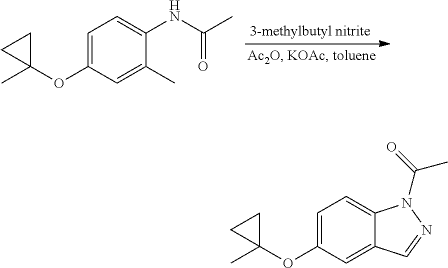 [SMILES: CC(=O)Nc1ccc(OC2(C)CC2)cc1C ; CC(=O)n1ncc2cc(OC3(C)CC3)ccc21]

To a solution of N-[2-methyl-4-(1-methylcyclopropoxy)phenyl]acetamide (9.3 g, 42.41 mmol, 1 eq) in toluene (100 mL) was added KOAc (6.24 g, 63.62 mmol, 1.5 eq) and Ac₂O (19.92 g, 195.09 mmol, 18.27 mL, 4.6 eq) at 20° C., the solution was heated to 80° C., then 3-methylbutyl nitrite (19.87 g, 169.65 mmol, 22.84 mL, 4 eq) was added dropwise. After addition, the mixture was stirred at 80° C. for 2 h. TLC showed the reaction was completed. The reaction was then filtered, the wet cake was washed with EtOAc (70 mL), and the filtrate was concentrated in vacuum. The residue was purified by silica gel chromatography (100-200 mesh silica gel, 0-10% Ethyl acetate in Petroleum ether) to afford 1-[5-(1-methylcyclopropoxy) indazol-1-yl]ethanone (8 g, crude) as a yellow solid.

### Step 6

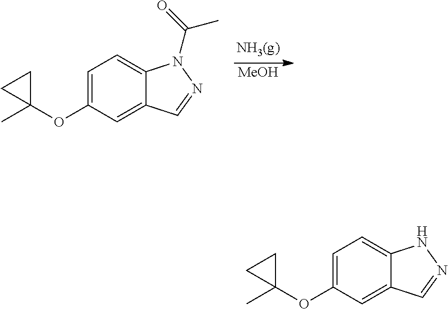 [SMILES: "MeOH" ; CC(=O)n1ncc2cc(OC3(C)CC3)ccc21 ; CC1(Oc2ccc3[nH]ncc3c2)CC1]

To a mixture of 1-[5-(1-methylcyclopropoxy)indazol-1-yl]ethanone (8 g, 34.74 mmol, 1 eq) in MeOH (80 mL) was added NH₃(g/)MeOH (7 M, 24.82 mL, 5 eq) in one portion at 20° C. The mixture was stirred at 20° C. for 2 hours to give a yellow solution. TLC showed the reaction was completed. The solution was concentrated in vacuum to afford 5-(1-methylcyclopropoxy)-1H-indazole (7.8 g, crude) as a yellow solid.

### Step 7

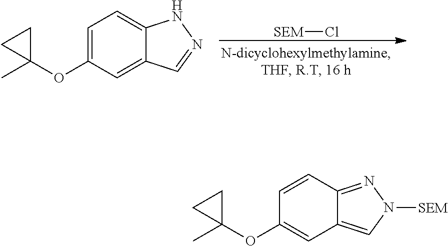 [SMILES: CC1(Oc2ccc3[nH]ncc3c2)CC1 ; C[Si](C)(C)CCOCCl ; CC1(Oc2ccc3nn(COCC[Si](C)(C)C)cc3c2)CC1]

To a mixture of 5-(1-methylcyclopropoxy)-1H-indazole (7.8 g, 41.44 mmol, 1 eq) in THF (80 mL) was added N-dicyclohexylmethylamine (10.52 g, 53.87 mmol, 1.3 eq) and SEM-CI (8.29 g, 49.73 mmol, 8.80 mL, 1.2 eq) in one portion at 20° C. The mixture was stirred at 20° C. for 16 hours to give an orange solution. TLC showed the reaction was completed. The residue was poured into water (60 mL). The aqueous phase was extracted with ethyl acetate (3×50 mL). The combined organic phase was washed with brine (2×50 mL), dried with anhydrous Na₂SO₄, filtered and concentrated in vacuum. The residue was purified by silica gel chromatography (100-200 mesh silica gel, 0-10% of ethyl acetate in Petroleum ether) to afford trimethyl-[2-[[5-(1-methylcyclopropoxy) indazol-2-yl]methoxy]ethyl]silane (5.4 g, 16.96 mmol, 40.92% yield) as a yellow oil.

### Step 8

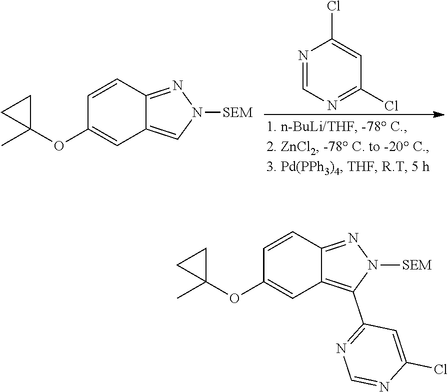 [SMILES: CC1(Oc2ccc3nn(COCC[Si](C)(C)C)cc3c2)CC1 ; Clc1cc(Cl)ncn1 ; CC1(Oc2ccc3nn(COCC[Si](C)(C)C)c(-c4cc(Cl)ncn4)c3c2)CC1]

To a mixture of trimethyl-[2-[[5-(1-methylcyclopropoxy)indazol-2-yl]methoxy]ethyl]silane (4.36 g, 13.70 mmol, 5.32e-1 eq) in THF (6 mL) was dropwise added n-BuLi (2.5 M, 13.40 mL, 1.3 eq) dropwise at −70° C. under N₂. The mixture was then stirred at −20° C. for 1 h, and a solution of ZnCl₂ (0.7 M, 55.20 mL, 1.5 eq) was dropwise added at −70° C. The mixture was stirred for 1 h at −40° C. A mixture of 4,6-dichloropyrimidine (4.22 g, 28.34 mmol, 1.1 eq) and Pd(PPh₃)₄ (1.49 g, 1.29 mmol, 0.05 eq) in THF (4 mL) was stirred at 20° C. for 1 h and was added to that solution. The cold bath was removed, and the mixture was stirred at 20° C. for 16 h to give a yellow solution. TLC showed there was starting material remained and at the same time some new spots were formed. The residue was poured into water (10 mL). The aqueous phase was extracted with ethyl acetate (3×20 mL). The combined organic phase was washed with brine (2×20 mL), dried with anhydrous Na₂SO₄, filtered and concentrated in vacuum. The residue was purified by silica gel chromatography (100-200 mesh silica gel, 0-10% of Ethyl acetate in Petroleum ether) to afford 2-[[3-(6-chloropyrimidin-4-yl)-5-(1-methylcyclopropoxy) indazol-2-yl]methoxy]ethyl-trimethyl-silane (2.9 g, crude) as a yellow oil.

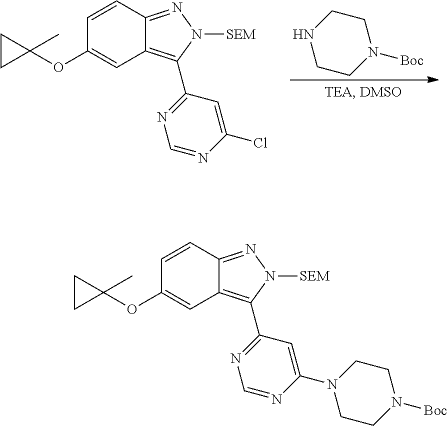 [SMILES: CC1(Oc2ccc3nn(COCC[Si](C)(C)C)c(-c4cc(Cl)ncn4)c3c2)CC1 ; CC(C)(C)OC(=O)N1CCNCC1 ; CC(C)(C)OC(=O)N1CCN(c2cc(-c3c4cc(OC5(C)CC5)ccc4nn3COCC[Si](C)(C)C)ncn2)CC1]

To a solution of 2-[[3-(6-chloropyrimidin-4-yl)-5-(1-methylcyclopropoxy)indazol-2-yl]methoxy]ethyl-trimethyl-silane (1 g, 2.32 mmol, 1 eq) and tert-butyl piperazine-1-carboxylate (648.20 mg, 3.48 mmol, 1.5 eq) in DMSO (5 mL) was added Et3N (704.34 mg, 6.96 mmol, 968.82 uL, 3 eq). After addition, the reaction mixture was stirred at 100° C. for 1 h. After cooling, the reaction mixture was diluted with water (10 mL) and extracted with ethyl acetate (3×10 mL). The organic layer was dried over sodium sulfate and concentrated under reduced pressure. The residue was purified by silica gel column chromatography (0 to 50% ethyl acetate in petroleum ether) to afford tert-butyl 4-[6-[5-(1-methylcyclopropoxy)-2-(2-trimethylsilylethoxymethyl)indazol-3-yl]pyrimidin-4-yl]piperazine-1-carboxylate (1.2 g, 1.96 mmol, 84.60% yield, 95% purity) as a light yellow solid.

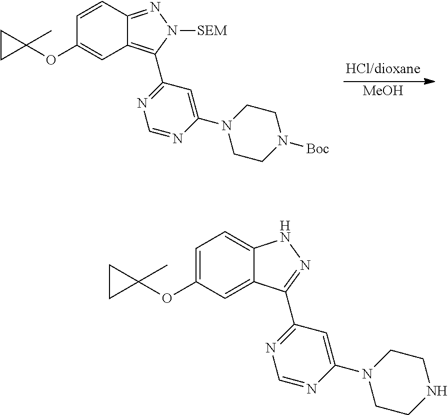 [SMILES: "MeOH" ; CC(C)(C)OC(=O)N1CCN(c2cc(-c3c4cc(OC5(C)CC5)ccc4nn3COCC[Si](C)(C)C)ncn2)CC1 ; CC1(Oc2ccc3[nH]nc(-c4cc(N5CCNCC5)ncn4)c3c2)CC1]

### Step 10

To a solution of tert-butyl 4-[6-[5-(1-methylcyclopropoxy)-2-(2-trimethylsilylethoxymethyl)indazol-3-yl]pyrimidin-4-yl]piperazine-1-carboxylate (1.2 g, 2.07 mmol, 1 eq) in MeOH (5 mL) was added HCl/dioxane (4 M, 5 mL, 9.68 eq). After addition, the reaction solution was stirred at 65° C. for 1 h. After cooling, the reaction mixture was concentrated under reduced pressure to afford 5-(1-methylcyclopropoxy)-3-(6-piperazin-1-ylpyrimidin-4-yl)-1H-indazole (770 mg, 1.81 mmol, 87.37% yield, 90.7% purity, HCl) as a yellow solid. The crude product was used for next step directly.

### Exemplary Synthesis of Intermediate 2: 3-(4,5-difluoro-1-oxo-isoindolin-2-yl)piperidine-2,6-dione

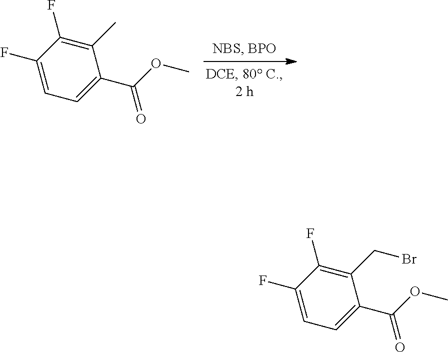

### Step 1:

To a mixture of methyl 3,4-difluoro-2-methyl-benzoate (40.00 g, 214.87 mmol, 1 eq) in 1,2-dichloroethane (400 mL) was added n-bromosuccinimide (57.37 g, 322.31 mmol, 1.5 eq) and benzoyl peroxide (520 mg, 2.15 mmol, 0.01 eq). The mixture was degassed and purged with nitrogen for 3 times, and then the mixture was stirred at 80° C. for 2 h. Thin layer chromatography indicated the reaction was completed. The mixture was cooled to 20° C., then it was filtered and concentrated under reduce pressure to give a residue. The residue was purified by silica gel column chromatography (petroleum ether:ethyl acetate=1:0 to 20:1) to give methyl 2-(bromomethyl)-3,4-difluoro-benzoate (51.00 g, 192.42 mmol, 89% yield) as a colorless oil.

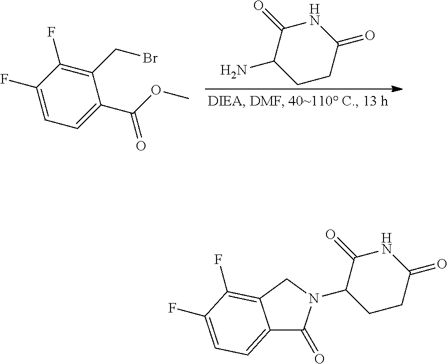 [SMILES: COC(=O)c1ccc(F)c(F)c1CBr ; NC1CCC(=O)NC1=O ; O=C1CCC(N2Cc3c(ccc(F)c3F)C2=O)C(=O)N1]

### Step 2:

To a mixture of methyl 2-(bromomethyl)-3,4-difluoro-benzoate (51.00 g, 192.42 mmol, 1 eq) and 3-aminopiperidine-2,6-dione (33.25 g, 202.04 mmol, 1.05 eq, hydrochloride) in N,N-dimethylformamide (600 mL) was added diisopropylethylamine (74.61 g, 577.25 mmol, 100.5 mL, 3 eq). The mixture was stirred at 40° C. for 1 h, and then heated to 110° C. for 12 h. Thin layer chromatography indicated the reaction was completed. The mixture was poured into water (800 mL), filtered and the solid was obtained. solid was dissolved in ethyl acetate (500 mL), filtered and the solid was obtained. Compound 3-(4,5-difluoro-1-oxo-isoindolin-2-yl)piperidine-2,6-dione (29.70 g, 105.99 mmol, 55% yield) was obtained as a gray solid.

### Exemplary Synthesis of Exemplary Compound 1:

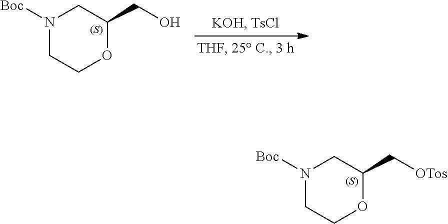

### Step 1:

To a mixture of tert-butyl (2S)-2-(hydroxymethyl)morpholine-4-carboxylate (5 g, 23.01 mmol, 1 eq) and potassium hydroxide (6.46 g, 115.07 mmol, 5 eq) in tetrahydrofuran (50 mL) was added 4-methylbenzenesulfonyl chloride (5.27 g, 27.62 mmol, 1.2 eq) in one portion at 25° C. The mixture was stirred at 25° C. for 3 hours. The mixture was added water (50 mL), then extracted with ethyl acetate (80 mL×3). The combined organic phase was washed with brine (30 mL×2), dried with anhydrous sodium sulfate, filtered and concentrated in vacuum. Compound tert-butyl (2S)-2-(p-tolylsulfonyloxymethyl)morpholine-4-carboxylate (8.5 g, 22.88 mmol, 99% yield) was obtained as a white oil into the next step without further purification.

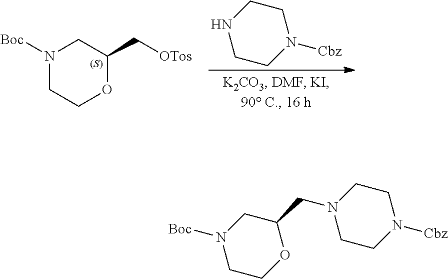

### Step 2:

To a mixture of tert-butyl (2S)-2-(p-tolylsulfonyloxymethyl)morpholine-4-carboxylate (3 g, 8.08 mmol, 1 eq), potassium carbonate (2.23 g, 16.15 mmol, 2 eq) and benzyl piperazine-1-carboxylate (5.34 g, 24.23 mmol, 4.68 mL, 3 eq) in dimethylformamide (30 mL) was added potassium iodide (2.68 g, 16.15 mmol, 2 eq) in one portion at 25° C. The mixture was stirred at 90° C. for 16 h and cooled. Water (50 mL) was added and the mixture was then extracted with ethyl acetate (50 mL×3). The combined organic phase was washed with brine (30 mL×2), dried with anhydrous sodium sulfate, filtered, and concentrated under vacuum. The residue was purified by column chromatography (Petroleum ether/Ethyl acetate=10/1 to 1). Compound tert-butyl (2R)-2-[(4-benzyloxycarbonylpiperazin-1-yl)methyl]morpholine-4-carboxylate (2.5 g, 5.96 mmol, 73% yield) was obtained as a yellow oil.

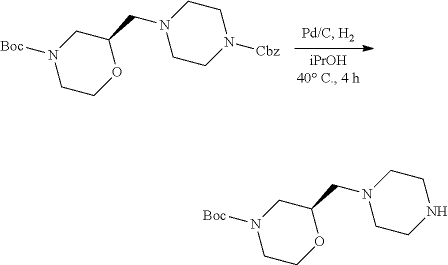 [SMILES: CC(C)(C)OC(=O)N1CCO[C@H](CN2CCN(C(=O)OCc3ccccc3)CC2)C1 ; CC(C)(C)OC(=O)N1CCO[C@H](CN2CCNCC2)C1]

### Step 3:

To a solution of tert-butyl (2R)-2-[(4-benzyloxycarbonylpiperazin-1-yl)methyl]morpholine-4-carboxylate (1 g, 2.38 mmol, 1 eq) in Isopropyl alcohol (20 mL) was added palladium on carbon (100 mg, 10% purity, 1.00 eq) under nitrogen. The suspension was degassed under vacuum and purged with hydrogen several times. The mixture was stirred under hydrogen (15 Psi) at 40° C. for 4 h. The reaction mixture was filtered and concentrated to give a product. Compound tert-butyl (2R)-2-(piperazin-1-ylmethyl)morpholine-4-carboxylate (0.63 g, 2.21 mmol, 92% yield) was obtained as a white oil into the next step without further purification.

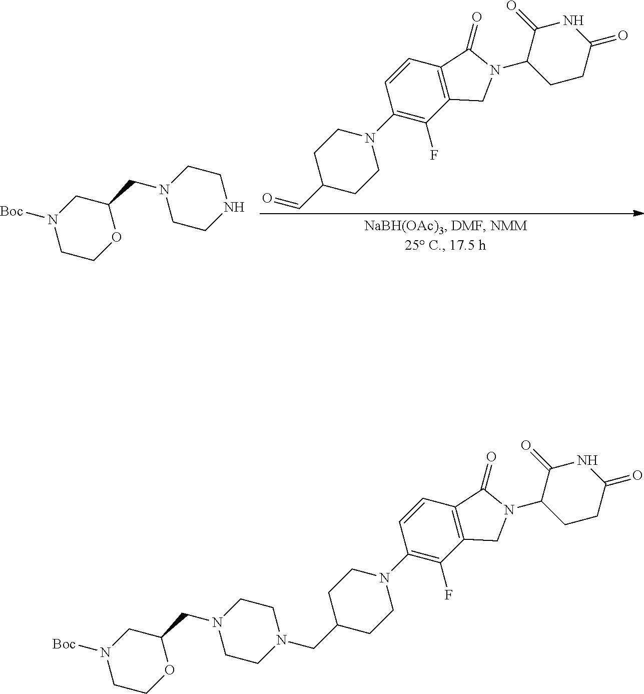

### Step 4:

To a solution of 1-[2-(2,6-dioxo-3-piperidyl)-4-fluoro-1-oxo-isoindolin-5-yl]piperidine-4-carbaldehyde (250 mg, 0.67 mmol, 1 eq) in dimethylformamide (3 mL) was added tert-butyl (2R)-2-(piperazin-1-ylmethyl)morpholine-4-carboxylate (248.41 mg, 0.87 mmol, 1.3 eq) in one portion at 25° C. The mixture was stirred at 25° C. for 0.5 h. Then sodium cyanoborohydride (84.15 mg, 1.34 mmol, 2 eq) was added into the mixture and stirred at 25° C. for 16 h. Water (5 mL) was added to the reaction solution, then the mixture was filtered to get the filter cake. The filtered cake was purified by prep-HPLC (column: 3_Phenomenex Luna C18 75*30 mm*3 um; mobile phase: [water(Trifluoroacetic acid)-acetonitrile]; B %: 17%-37%, 9 min). Compound tert-butyl (2R)-2-[[4-[[1-[2-(2,6-dioxo-3-piperidyl)-4-fluoro-1-oxo-isoindolin-5-yl]-4-piperidyl]methyl]piperazin-1-yl]methyl]morpholine-4-carboxylate (0.22 g, 0.34 mmol, 51% yield) was obtained as a white solid.

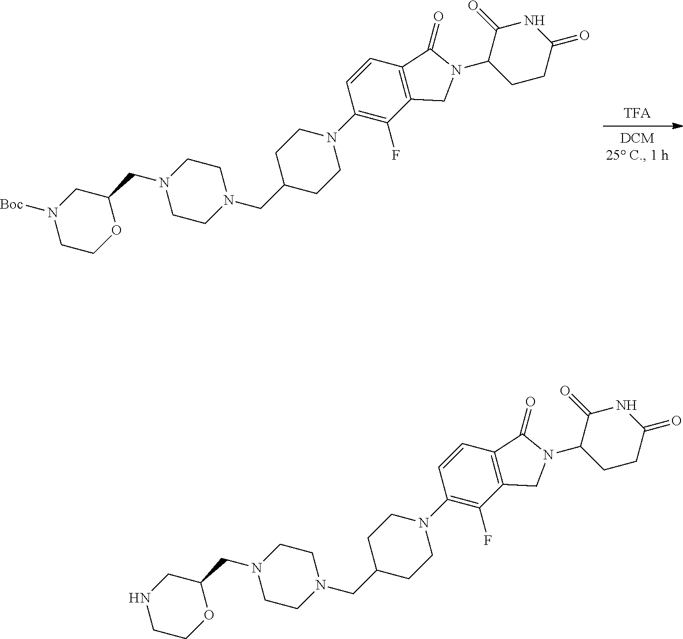 [SMILES: CC(C)(C)OC(=O)N1CCO[C@H](CN2CCN(CC3CCN(c4ccc5c(c4F)CN(C4CCC(=O)NC4=O)C5=O)CC3)CC2)C1 ; O=C1CCC(N2Cc3c(ccc(N4CCC(CN5CCN(C[C@@H]6CNCCO6)CC5)CC4)c3F)C2=O)C(=O)N1]

### Step 5:

To a mixture of tert-butyl (2R)-2-[[4-[[1-[2-(2,6-dioxo-3-piperidyl)-4-fluoro-1-oxo-isoindolin-5-yl]-4-piperidyl]methyl]piperazin-1-yl]methyl]morpholine-4-carboxylate (205 mg, 0.31 mmol, 1 eq) in dichloromethane (4 mL) was added trifluoroacetic acid (1.54 g, 13.51 mmol, 1.00 mL, 42.3 eq) in one portion at 25° C. The mixture was stirred at 25° C. for 1 hour. The reaction was concentrated under vacuum to afford 3-[4-fluoro-5-[4-[[4-[[(2S)-morpholin-2-yl]methyl]piperazin-1-yl]methyl]-1-piperidyl]-1-oxo-isoindolin-2-yl]piperidine-2,6-dione (170 mg, 0.31 mmol, 98% yield) as a white oil which was used directly in the next step without further purification.

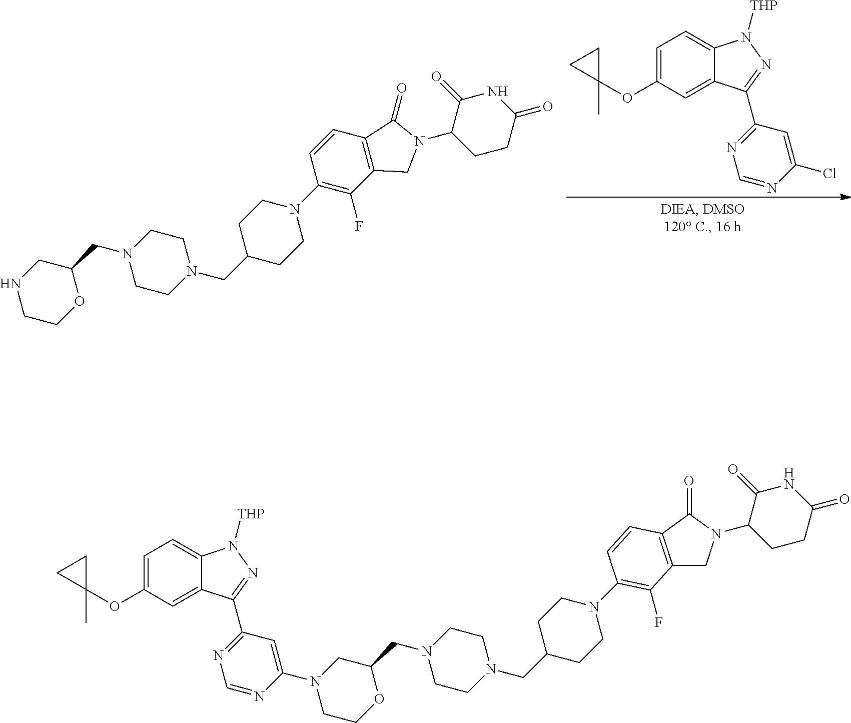

### Step 6:

To a mixture of 3-[4-fluoro-5-[4-[[4-[[(2S)-morpholin-2-yl]methyl]piperazin-1-yl]methyl]-1-piperidyl]-1-oxo-isoindolin-2-yl]piperidine-2,6-dione (170 mg, 0.31 mmol, 1 eq) and 3-(6-chloropyrimidin-4-yl)-5-(1-methylcyclopropoxy)-1-tetrahydropyran-2-yl-indazole (132.63 mg, 0.34 mmol, 1.1 eq) in dimethylsulfoxide (3 mL) was added diisopropylethylamine (161.95 mg, 1.25 mmol, 0.2 mL, 4 eq) in one portion at 25° C. The mixture was stirred at 120° C. for 16 h and cooled. Adding 5 mL of water to the reaction produced a solid, which was then filtered. The resulting material was purified by prep-HPLC (column: Phenomenex Synergi Polar-RP 100*25 mm*4 um; mobile phase: [water(Trifluoroacetic acid)-acetonitrile]; B %: 32%-52%, 9 min). Compound 3-[4-fluoro-5-[4-[[4-[[(2R)-4-[6-[5-(1-methylcyclopropoxy)-1-tetrahydropyran-2-yl-indazol-3-yl]pyrimidin-4-yl]morpholin-2-yl]methyl]piperazin-1-yl]methyl]-1-piperidyl]-1-oxo-isoindolin-2-yl]piperidine-2,6-dione (0.13 g, 0.14 mmol, 46% yield) was obtained as a white solid.

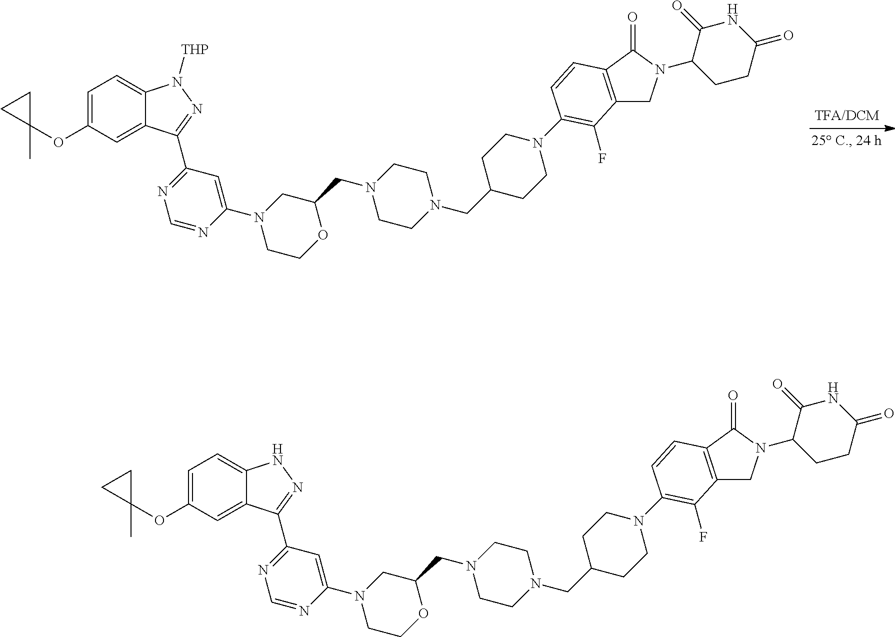

### Step 7:

To a mixture of 3-[4-fluoro-5-[4-[[4-[[(2R)-4-[6-[5-(1-methylcyclopropoxy)-1-tetrahydropyran-2-yl-indazol-3-yl]pyrimidin-4-yl]morpholin-2-yl]methyl]piperazin-1-yl]methyl]-1-piperidyl]-1-oxo-isoindolin-2-yl]piperidine-2,6-dione (120 mg, 0.13 mmol, 1 eq) in dichloromethane (2 mL) was added trifluoroacetic acid (2.31 g, 20.26 mmol, 1.5 mL, 150.43 eq) in one portion at 25° C. The mixture was stirred at 25° C. for 24 h and then concentrated under reduced pressure to remove solvent. The residue was purified by prep-HPLC (column: Unisil 3-100 C18 Ultra 150*50 mm*3 um; mobile phase: [water(formic acid)-acetonitrile]; B %: 11%-41%, 7 min). Compound 3-[4-fluoro-5-[4-[[4-[[(2R)-4-[6-[5-(1-methylcyclopropoxy)-1H-indazol-3-yl]pyrimidin-4-yl]morpholin-2-yl]methyl]piperazin-1-yl]methyl]-1-piperidyl]-1-oxo-isoindolin-2-yl]piperidine-2,6-dione (44.7 mg, 0.05 mmol, 41% yield, formate) was obtained as a white solid.

Exemplary Synthesis of Exemplary Compound 2: Compound 2 was prepared in a manner analogous to compound 1 starting with (2R)-2-(hydroxymethyl)morpholine-4-carboxylate.

Exemplary Synthesis of Exemplary Compound 3: Compound 3 was prepared in a manner analogous to compound 1.

### Exemplary Synthesis of Exemplary Compound 4:

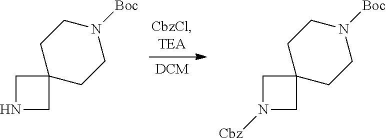 [SMILES: CC(C)(C)OC(=O)N1CCC2(CC1)CNC2 ; CC(C)(C)OC(=O)N1CCC2(CC1)CN(C(=O)OCc1ccccc1)C2]

### Step 1

To a solution of tert-butyl 2,7-diazaspiro[3.5]nonane-7-carboxylate (3 g, 13.26 mmol, 1 eq) in DCM (30 mL) was added CbzCl (2.88 g, 16.88 mmol, 2.4 mL, 1.27 eq) and TEA (4.02 g, 39.77 mmol, 5.54 mL, 3 eq) at 0° C. The mixture was stirred at 20° C. for 2 hr. The reaction mixture was diluted with H₂O (50 mL) and extracted with DCM (20 mL×3). The combined organic layers were washed with brine, dried over Na₂SO₄, filtered, and concentrated under reduced pressure to give a residue. The residue was purified by flash silica gel chromatography (ISCO®; 40 g SepaFlash® Silica Flash Column, Eluent of 0-32% Ethyl acetate/Petroleum ether gradient @ 100 mL/min) to give 02-benzyl 07-tert-butyl 2,7-diazaspiro[3.5]nonane-2,7-dicarboxylate (2.77 g, 7.68 mmol, 57.97% yield) as a colorless oil.

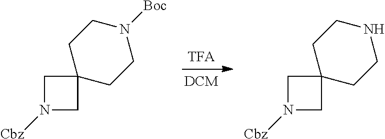 [SMILES: CC(C)(C)OC(=O)N1CCC2(CC1)CN(C(=O)OCc1ccccc1)C2 ; O=C(OCc1ccccc1)N1CC2(CCNCC2)C1]

### Step 2

To a solution of 02-benzyl 07-tert-butyl 2,7-diazaspiro[3.5]nonane-2,7-dicarboxylate (2.77 g, 7.68 mmol, 1 eq) in DCM (10 mL) was added TFA (7.70 g, 67.53 mmol, 5.00 mL, 8.79 eq). The mixture was stirred at 20° C. for 0.5 hr. The reaction mixture was concentrated under reduced pressure to remove DCM to give benzyl 2,7-diazaspiro[3.5]nonane-2-carboxylate (3.2 g, crude, TFA) as a yellow oil.

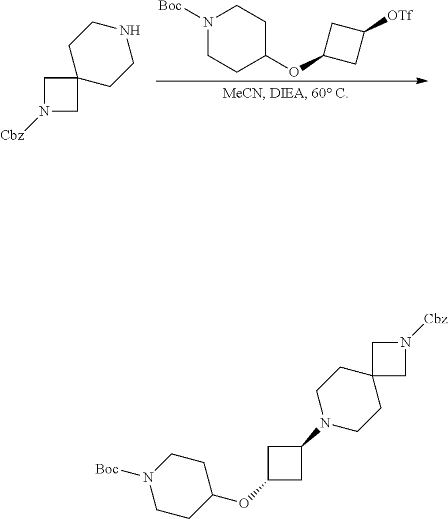 [SMILES: O=C(OCc1ccccc1)N1CC2(CCNCC2)C1 ; CC(C)(C)OC(=O)N1CCC(O[C@H]2C[C@@H](OS(=O)(=O)C(F)(F)F)C2)CC1 ; CC(C)(C)OC(=O)N1CCC(O[C@H]2C[C@H](N3CCC4(CC3)CN(C(=O)OCc3ccccc3)C4)C2)CC1]

### Step 3:

To a solution of benzyl 2,7-diazaspiro[3.5]nonane-2-carboxylate (1.25 g, 4.80 mmol, 1 eq) and tert-butyl 4-[3-(trifluoromethylsulfonyloxy)cyclobutoxy]piperidine-1-carboxylate (2.52 g, 6.24 mmol, 1.3 eq) in MeCN (10 mL) was added DIEA (3.10 g, 24.01 mmol, 4.18 mL, 5 eq). The mixture was stirred at 60° C. for 12 h and cooled. The reaction mixture was added silica powder and concentrated under reduced pressure to remove MeCN. The residue was purified by flash silica gel chromatography (ISCO®; 40 g SepaFlash® Silica Flash Column, Eluent of 0˜8% Methanol/Dichloromethane gradient @ 100 mL/min) to give a benzyl 7-[3-[(1-tert-butoxycarbonyl-4-piperidyl)oxy]cyclobutyl]-2,7-diazaspiro[3.5]nonane-2-carboxylate (2 g, 3.89 mmol, 81.09% yield) as a yellow solid.

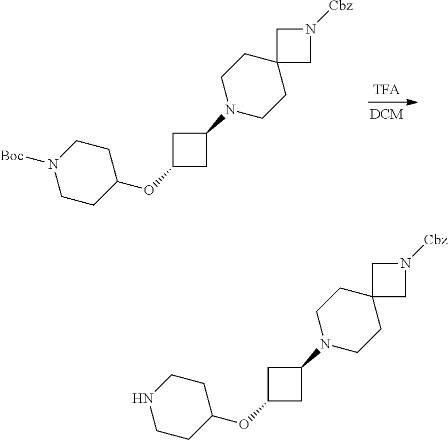 [SMILES: CC(C)(C)OC(=O)N1CCC(O[C@H]2C[C@H](N3CCC4(CC3)CN(C(=O)OCc3ccccc3)C4)C2)CC1 ; O=C(OCc1ccccc1)N1CC2(CCN([C@H]3C[C@H](OC4CCNCC4)C3)CC2)C1]

### Step 4:

To a solution of benzyl 7-[3-[(1-tert-butoxycarbonyl-4-piperidyl)oxy]cyclobutyl]-2,7-diazaspiro[3.5]nonane-2-carboxylate (770 mg, 1.50 mmol, 1 eq) in DCM (3 mL) was added TFA (854.62 mg, 7.50 mmol, 554.95 uL, 5 eq). The mixture was stirred at 20° C. for 1 h. The reaction mixture was concentrated under reduced pressure to remove DCM. The residue was diluted with H₂O (20 mL) and extracted with EtOAc (20 mL×1). The H₂O phase was added Na₂CO₃ to adjust pH>8 and extracted with DCM (20 mL×4). The combined organic layers were washed with brine, dried over Na₂SO₄, filtered and concentrated under reduced pressure to give a benzyl 7-[3-(4-piperidyloxy)cyclobutyl]-2,7-diazaspiro[3.5]nonane-2-carboxylate (300 mg, 725.42 umol, 48.39% yield) as a yellow oil.

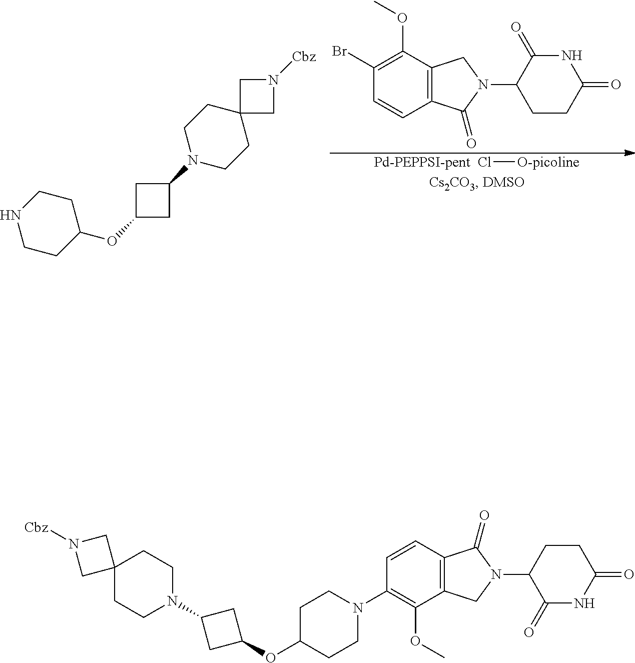

### Step 5:

To a solution of benzyl 7-[3-(4-piperidyloxy)cyclobutyl]-2,7-diazaspiro[3.5]nonane-2-carboxylate (200 mg, 483.62 umol, 1 eq) and 3-(5-bromo-4-methoxy-1-oxo-isoindolin-2-yl)piperidine-2,6-dione (204.96 mg, 580.34 umol, 1.2 eq) in DMSO (5 mL) was added Pd-PEPPSI-pent CI-O-picoline (26.26 mg, 48.36 umol, 0.1 eq) and Cs₂CO₃ (630.28 mg, 1.93 mmol, 4 eq). The mixture was stirred at 90° C. for 20 h and cooled. The mixture was purified by flash silica gel chromatography (ISCO®; 12 g SepaFlash® Silica Flash Column, Eluent of 0-100% Ethyl acetate/Petroleum ether gradient @ 100 mL/min) to give the crude product. The crude product was purified by prep-HPLC (column: Phenomenex C18 75*30 mm*3 um; mobile phase: [water(FA)-ACN]; B %: 18%-48%, 28 min) to give 160 mg crude product. The 160 mg crude product was purified by prep-HPLC (column: Phenomenex C18 75*30 mm*3 um; mobile phase: [water(TFA)-ACN]; B %: 8%-38%, 25 min) to give benzyl 7-[3-[[1-[2-(2,6-dioxo-3-piperidyl)-4-methoxy-1-oxo-isoindolin-5-yl]-4-piperidyl]oxy]cyclobutyl]-2,7-diazaspiro[3.5]nonane-2-carboxylate (50 mg, 62.70 umol, 12.96% yield, 86% purity) as a white solid.

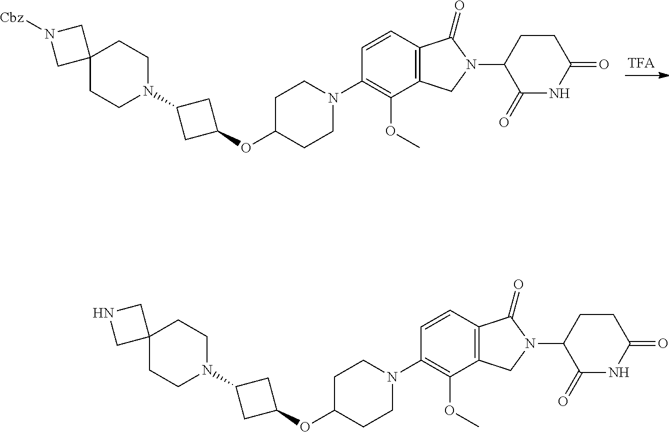 [SMILES: COc1c(N2CCC(O[C@H]3C[C@H](N4CCC5(CC4)CN(C(=O)OCc4ccccc4)C5)C3)CC2)ccc2c1CN(C1CCC(=O)NC1=O)C2=O ; COc1c(N2CCC(O[C@H]3C[C@H](N4CCC5(CC4)CNC5)C3)CC2)ccc2c1CN(C1CCC(=O)NC1=O)C2=O]

### Step 6:

To a solution of benzyl 7-[3-[[1-[2-(2,6-dioxo-3-piperidyl)-4-methoxy-1-oxo-isoindolin-5-yl]-4-piperidyl]oxy]cyclobutyl]-2,7-diazaspiro[3.5]nonane-2-carboxylate (50 mg, 72.91 umol, 1 eq) in TFA (24.94 mg, 218.72 umol, 16.19 uL, 3 eq). The mixture was stirred at 70° C. for 1 h and cooled. The reaction mixture was concentrated under reduced pressure to remove TFA to give a 3-[5-[4-[3-(2,7-diazaspiro[3.5]nonan-7-yl)cyclobutoxy]-1-piperidyl]-4-methoxy-1-oxo-isoindolin-2-yl]piperidine-2,6-dione (70 mg, crude, 4TFA) was obtained as a yellow oil.

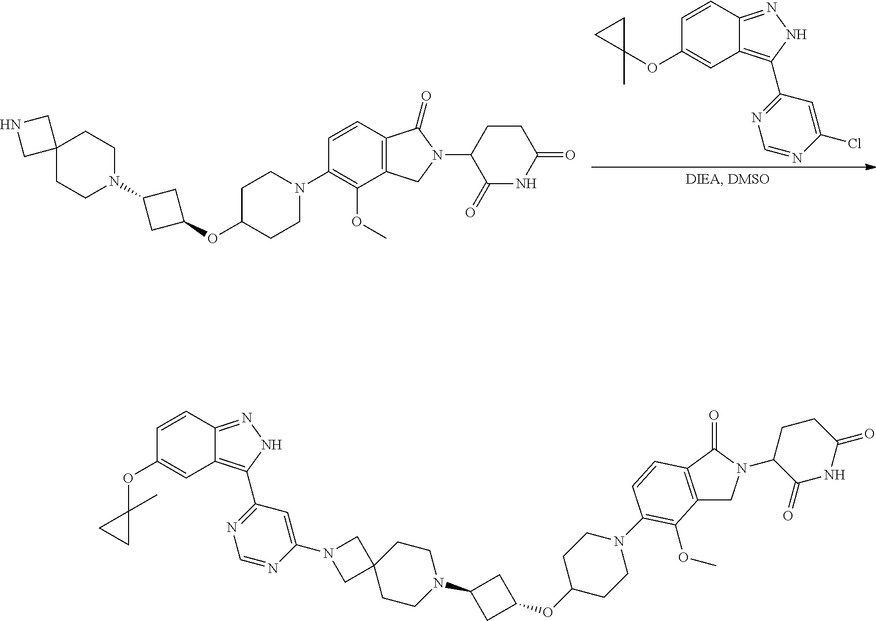 [SMILES: COc1c(N2CCC(O[C@H]3C[C@H](N4CCC5(CC4)CNC5)C3)CC2)ccc2c1CN(C1CCC(=O)NC1=O)C2=O ; CC1(Oc2ccc3n[nH]c(-c4cc(Cl)ncn4)c3c2)CC1 ; COc1c(N2CCC(O[C@H]3C[C@H](N4CCC5(CC4)CN(c4cc(-c6[nH]nc7ccc(OC8(C)CC8)cc67)ncn4)C5)C3)CC2)ccc2c1CN(C1CCC(=O)NC1=O)C2=O]

### Step 7:

To a solution of 3-[5-[4-[3-(2,7-diazaspiro[3.5]nonan-7-yl)cyclobutoxy]-1-piperidyl]-4-methoxy-1-oxo-isoindolin-2-yl]piperidine-2,6-dione (40 mg, 72.51 umol, 1 eq) and 3-(6-chloropyrimidin-4-yl)-5-(1-methylcyclopropoxy)-2H-indazole (21.81 mg, 72.51 umol, 1 eq) in DMSO (2 mL) was added DIEA (46.85 mg, 362.53 umol, 63.15 uL, 5 eq). The mixture was stirred at 80° C. for 12 h and cooled. The residue was purified by prep-HPLC (column: Phenomenex C18 75*30 mm*3 um; mobile phase: [water(FA)-ACN]; B %: 0%-30%, 25 min) to give 3-[4-methoxy-5-[4-[3-[2-[6-[5-(1-methylcyclopropoxy)-2H-indazol-3-yl]pyrimidin-4-yl]-2,7-diazaspiro[3.5]nonan-7-yl]cyclobutoxy]-1-piperidyl]-1-oxo-isoindolin-2-yl]piperidine-2,6-dione (20.5 mg, 25.07 umol, 34.58% yield, 99.794% purity) as a white solid.

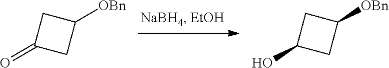 [SMILES: O=C1CC(OCc2ccccc2)C1 ; O[C@H]1C[C@@H](OCc2ccccc2)C1]

### Step 8:

To a solution of 3-benzyloxycyclobutanone (10 g, 56.75 mmol, 1 eq) in EtOH (50 mL) was added NaBH₄ (2.6 g, 68.73 mmol, 1.21 eq) at 0° C. under N₂. Then the mixture was stirred at 0° C. for 1 h to give colorless solution. TLC (Petroleum ether:Ethyl acetate=2:1) showed the starting material was consumed completely. The mixture was poured into HCl (100 mL, 2 M) and extracted with EtOAc (50 mL*2). The organic layer was washed with brine (50 mL), dried over anhydrous Na₂SO₄, filtered, and concentrated under vacuum. The residue was purified by silica gel chromatography (120 g, diameter: 100 mm, 100-200 mesh silica gel, 0-50% (5 min) of Ethyl acetate in Petroleum ether, 50% (5 min) of Ethyl acetate in Petroleum ether) to give 3-benzyloxycyclobutanol (9.7 g, 54.42 mmol, 95.90% yield) as a yellow oil.

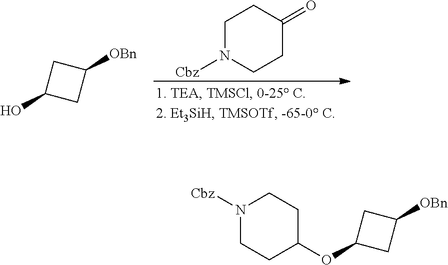 [SMILES: O[C@H]1C[C@@H](OCc2ccccc2)C1 ; O=C1CCN(C(=O)OCc2ccccc2)CC1 ; O=C(OCc1ccccc1)N1CCC(O[C@H]2C[C@@H](OCc3ccccc3)C2)CC1]

### Step 9:

To a solution of (1s,3s)-3-(benzyloxy)cyclobutanol (9.7 g, 54.42 mmol, 1 eq) in DCM (500 mL) was added TEA (6.33 g, 62.59 mmol, 8.71 mL, 1.15 eq) and TMSCI (6.50 g, 59.87 mmol, 7.60 mL, 1.1 eq) at 0° C., and the reaction mixture was stirred at 25° C. for 0.5 h. The reaction mixture was filtered and the filtrate was concentrated under reduced pressure to give a residue. Then to a stirred solution of above residue and benzyl 4-oxopiperidine-1-carboxylate (16.50 g, 70.75 mmol, 14.11 mL, 1.3 eq) in THF (500 mL) was added Et₃SiH (7.28 g, 62.59 mmol, 10.00 mL, 1.15 eq) and TMSOTf (604.82 mg, 2.72 mmol, 491.72 uL, 0.05 eq) dropwise at −60° C., and the reaction mixture was stirred at 0° C. under N₂ for 1.5 h. The reaction mixture was quenched by addition sat. NaHCO₃ adjust pH=8 at 0° C., and then diluted with extracted with Ethyl acetate (500 mL*3). The combined organic layers were washed with brine (500 mL), dried over Na₂SO₄, filtered, and concentrated under reduced pressure to give a residue. The residue was purified by silica gel chromatography (120 g, 100 mL/min, 0-30% (10 min) of Ethyl acetate in Petroleum ether, 30% (10 min) of Ethyl acetate in Petroleum ether) to give benzyl 4-(3-benzyloxycyclobutoxy)piperidine-1-carboxylate (20 g, 50.57 mmol, 92.92% yield) as a yellow oil.

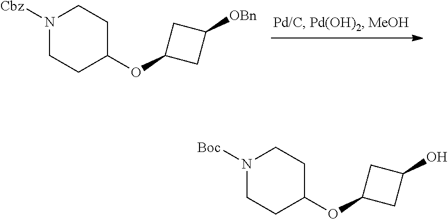 [SMILES: O=C(OCc1ccccc1)N1CCC(O[C@H]2C[C@@H](OCc3ccccc3)C2)CC1 ; CC(C)(C)OC(=O)N1CCC(O[C@H]2C[C@@H](O)C2)CC1]

### Step 10:

To a solution of benzyl 4-(3-benzyloxycyclobutoxy)piperidine-1-carboxylate (20 g, 50.57 mmol, 1 eq) and tert-butoxycarbonyl tert-butyl carbonate (13.46 g, 61.70 mmol, 14.17 mL, 1.22 eq) in MeOH (300 mL) was added Pd/C (3 g, 50.57 mmol, 10% purity, 1 eq) and Pd(OH)₂ (1.83 g, 1.30 mmol, 10% purity, 2.57e-2 eq) under N₂. The suspension was degassed under vacuum and purged with H₂ several times. The mixture was stirred under H₂ (50 psi) at 60° C. for 16 h. TLC (Petroleum ether:Ethyl acetate=0/1, RF=0.35) indicated no reactant remained and one major new spot was detected. The reaction mixture was filtered and the filtrate was concentrated. The crude product was purified by column chromatography on silica gel (column height: 80 g, 100-200 mesh silica gel, 0-100% (30 min) of Ethyl acetate in Petroleum ether) to give tert-butyl 4-(3-hydroxycyclobutoxy)piperidine-1-carboxylate (9.8 g, 36.12 mmol, 71.42% yield) as a colorless oil.

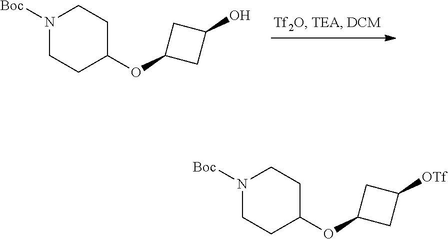 [SMILES: CC(C)(C)OC(=O)N1CCC(O[C@H]2C[C@@H](O)C2)CC1 ; CC(C)(C)OC(=O)N1CCC(O[C@H]2C[C@@H](OS(=O)(=O)C(F)(F)F)C2)CC1]

### Step 11:

To a solution of tert-butyl 4-(3-hydroxycyclobutoxy)piperidine-1-carboxylate (1 g, 3.69 mmol, 1 eq) in DCM (15 mL) was added TEA (372.91 mg, 3.69 mmol, 512.94 uL, 1 eq) at −40° C. and then Tf₂O (1.04 g, 3.69 mmol, 608.04 uL, 1 eq) was added at −40° C. under N₂, The mixture was stirred at −40° C. for 0.5 h. TLC (Petroleum ether:Ethyl acetate=1:1) indicated the reactant was consumed completely. The reaction mixture was diluted with H₂O (20 mL) and extracted with DCM (20 mL*3). The combined organic layers were washed with brine, dried over Na₂SO₄, filtered, and concentrated under reduced pressure to give a crude product. The residue was purified by silica gel chromatography (0-20% of Ethyl acetate in Petroleum ether) to give tert-butyl 4-[3-(trifluoromethylsulfonyloxy)cyclobutoxy]piperidine-1-carboxylate (500 mg, 1.24 mmol, 33.63% yield) as a white solid.

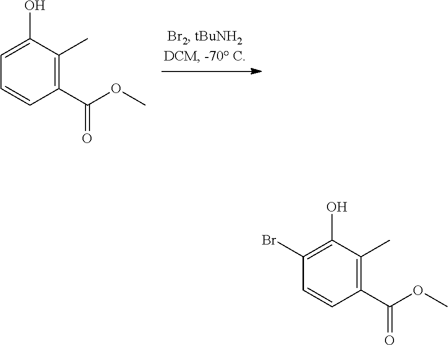

### Step 12:

To a solution of 2-methylpropan-2-amine (8.80 g, 120.36 mmol, 12.65 mL, 1 eq) in DCM (50 mL) at −70° C. was added a solution of Br₂ (19.23 g, 120.36 mmol, 6.20 mL, 1 eq) in DCM (50 mL) dropwise and the mixture was stirred at −70° C. for 1 h. A solution of methyl 3-hydroxy-2-methyl-benzoate (20 g, 120.36 mmol, 1 eq) in DCM (200 mL) was then added dropwise and the resulting mixture allowed to warm to 20° C. and stirred for 8 h. TLC (petroleum ether:ethyl acetate=5:1) indicated the reaction was consumed completely. The mixture was quenched with water (100 mL), extracted with DCM (500 mL), washed with brine (500 mL), dried over Na₂SO₄, and concentrated under reduced pressure. It was purified by silica gel chromatography (0-3% EtOAc in Petroleum ether) to afford methyl 4-bromo-3-hydroxy-2-methyl-benzoate (7.77 g, 31.71 mmol, 26.34% yield) as a yellow solid.

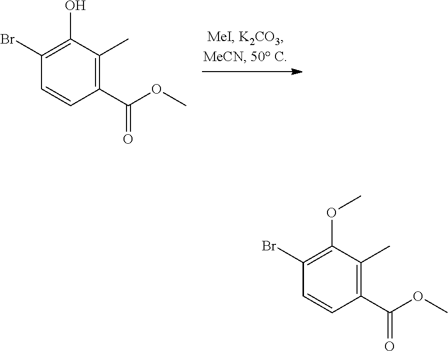

### Step 13:

To a solution of methyl 4-bromo-3-hydroxy-2-methyl-benzoate (7.77 g, 31.71 mmol, 1 eq) in MeCN (70 mL) was added Mel (13.50 g, 95.12 mmol, 5.92 mL, 3 eq) and K₂CO₃ (5.26 g, 38.05 mmol, 1.2 eq). The mixture was stirred at 50° C. for 5 h and cooled. TLC (Petroleum ether:Ethyl acetate=20:1) indicated the reaction was consumed completely. The mixture was quenched with water (10 mL), extracted with EtOAc (50 mL), washed with brine (50 mL), dried over Na₂SO₄, and concentrated under reduced pressure. It was purified by silica gel chromatography (0-3% EtOAc in Petroleum ether) to afford methyl 4-bromo-3-methoxy-2-methyl-benzoate (7.44 g, 28.72 mmol, 90.57% yield) as yellow oil.

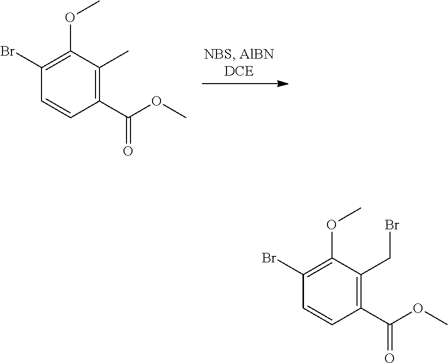

### Step 14:

To a stirred solution of methyl 4-bromo-3-methoxy-2-methyl-benzoate (2 g, 7.72 mmol, 1 eq) in DCE (10 mL) under an atmosphere of nitrogen was added NBS (1.65 g, 9.26 mmol, 1.2 eq) followed by 2-[(E)-(1-cyano-1-methyl-ethyl)azo]-2-methyl-propanenitrile (63.38 mg, 385.96 umol, 0.05 eq) and the resulting mixture was stirred vigorously at 80° C. for 1 h to give yellow solution. The reaction mixture was concentrated under reduced pressure. The residue was purified by silica gel chromatography (10% of Ethyl acetate in Petroleum ether) to give methyl 4-bromo-2-(bromomethyl)-3-methoxy-benzoate (2.3 g, 6.80 mmol, 88.16% yield) as a yellow oil.

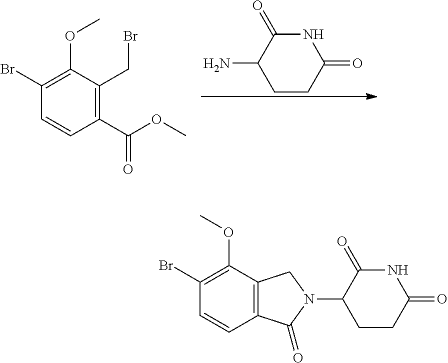 [SMILES: COC(=O)c1ccc(Br)c(OC)c1CBr ; NC1CCC(=O)NC1=O ; COc1c(Br)ccc2c1CN(C1CCC(=O)NC1=O)C2=O]

### Step 15

To a mixture of methyl 4-bromo-2-(bromomethyl)-3-methoxy-benzoate (2.3 g, 6.80 mmol, 1 eq) and 3-aminopiperidine-2,6-dione (1.34 g, 8.17 mmol, 1.2 eq, HCl) in DMF (10 mL) was added DIEA (4.40 g, 34.02 mmol, 5.93 mL, 5 eq) in one portion at 20° C. under N₂. The mixture was stirred at 85° C. for 48 h and cooled. The reaction mixture was concentrated under vacuum. The crude product was triturated with MeCN (20 mL) and H₂O (20 mL). The crude was filtered and the resulting solid was dried under vacuum to give 3-(5-bromo-4-methoxy-1-oxo-isoindolin-2-yl)piperidine-2,6-dione (2 g, 5.66 mmol, 83.22% yield) as a dark gray solid

Exemplary Synthesis of Exemplary Compound 5: Compound 5 was prepared in a manner analogous to compound 1.

### Exemplary Synthesis of Exemplary Compound 6:

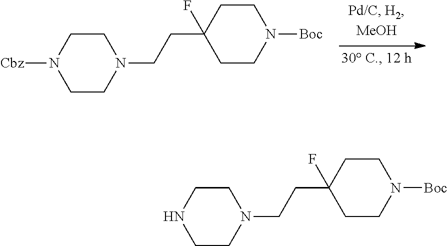 [SMILES: CC(C)(C)OC(=O)N1CCC(F)(CCN2CCN(C(=O)OCc3ccccc3)CC2)CC1 ; CC(C)(C)OC(=O)N1CCC(F)(CCN2CCNCC2)CC1]

### Step 1:

To a solution of benzyl 4-[2-(1-tert-butoxycarbonyl-4-fluoro-4-piperidyl)ethyl]piperazine-1-carboxylate (100 mg, 0.22 mmol, 1 eq) in methanol (10 mL) was added palladium/carbon (10 mg, 10% purity) under nitrogen. The suspension was degassed under vacuum and purged with hydrogen several times. The mixture was stirred under Hydrogen (15 psi) at 30° C. for 12 h. TLC (Dichloromethane:Methanol=10:1) showed the reaction was completed. The reaction mixture was filtered and concentrated under reduced pressure to give a residue. Compound tert-butyl 4-fluoro-4-(2-piperazin-1-ylethyl)piperidine-1-carboxylate (79 mg, crude) was obtained as a colorless oil.

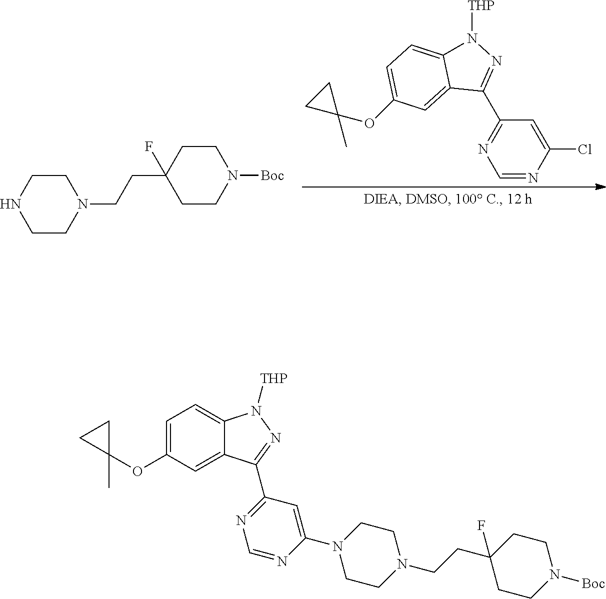

### Step 2:

To a solution of 3-(6-chloropyrimidin-4-yl)-5-(1-methylcyclopropoxy)-1-tetrahydropyran-2-yl-indazole (85 mg, 0.22 mmol, 1 eq) in dimethylsulfoxide (2 mL) was added diisopropylethylamine (86 mg, 0.67 mmol, 0.12 mL, 3 eq) and tert-butyl 4-fluoro-4-(2-piperazin-1-ylethyl)piperidine-1-carboxylate (70 mg, 0.22 mmol, 1 eq) and the mixture was stirred at 100° C. for 12 h and cooled. The reaction mixture was quenched by water (10 mL) and extracted with ethyl acetate (20 mL×3), the combined organic layers were washed with brine (30 mL×3), dried over anhydrous sodium sulfate, filtered, and concentrated under reduced pressure to give a residue. The residue was purified by prep-TLC (SiO2, dichloromethane:methanol=10:1). Compound tert-butyl 4-fluoro-4-[2-[4-[6-[5-(1-methylcyclopropoxy)-1-tetrahydropyran-2-yl-indazol-3-yl]pyrimidin-4-yl]piperazin-1-yl]ethyl]piperidine-1-carboxylate (60 mg, 0.09 mmol, 40% yield, 99% purity) was obtained as a colorless oil.

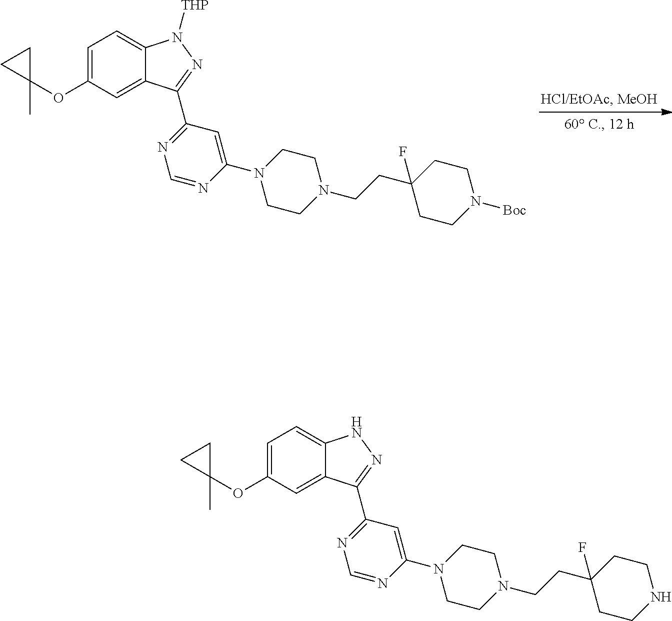

### Step 3:

To a solution of tert-butyl 4-fluoro-4-[2-[4-[6-[5-(1-methylcyclopropoxy)-1-tetrahydropyran-2-yl-indazol-3-yl]pyrimidin-4-yl]piperazin-1-yl]ethyl]piperidine-1-carboxylate (60 mg, 0.09 mmol, 1 eq) in methanol (1 mL) and hydrochloric acid/ethyl acetate (4 M, 1 mL, 44.25 eq) was stirred at 60° C. for 12 h and cooled. The reaction mixture was concentrated under reduced pressure to give a residue. Compound 3-[6-[4-[2-(4-fluoro-4-piperidyl)ethyl]piperazin-1-yl]pyrimidin-4-yl]-5-(1-methylcyclopropoxy)-1H-indazole (53 mg, 0.09 mmol, 98% yield, trifluoroacetate) was obtained as a yellow oil.

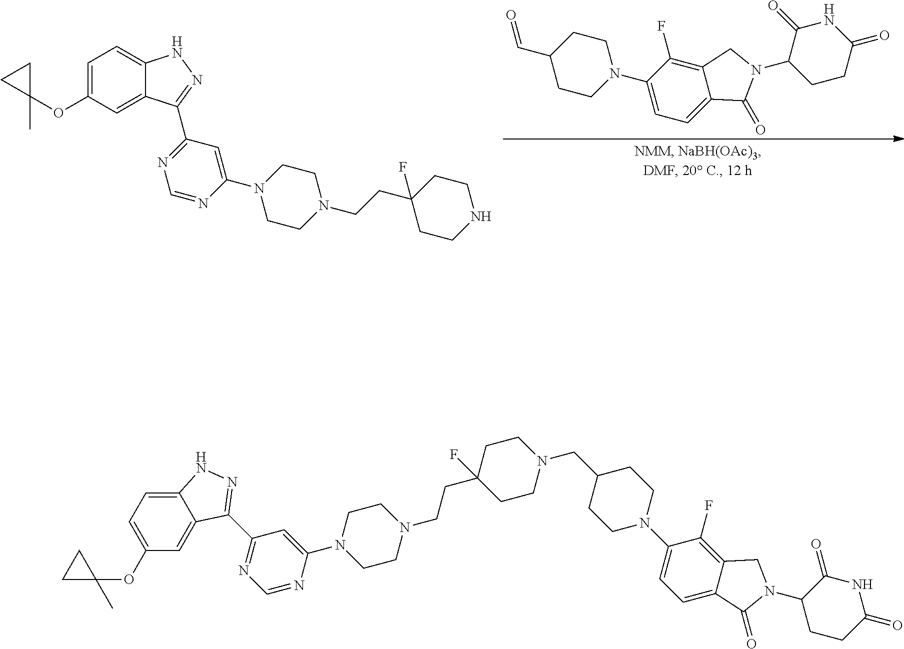 [SMILES: CC1(Oc2ccc3[nH]nc(-c4cc(N5CCN(CCC6(F)CCNCC6)CC5)ncn4)c3c2)CC1 ; O=CC1CCN(c2ccc3c(c2F)CN(C2CCC(=O)NC2=O)C3=O)CC1 ; CC1(Oc2ccc3[nH]nc(-c4cc(N5CCN(CCC6(F)CCN(CC7CCN(c8ccc9c(c8F)CN(C8CCC(=O)NC8=O)C9=O)CC7)CC6)CC5)ncn4)c3c2)CC1]

### Step 4:

To a solution of 1-[2-(2,6-dioxo-3-piperidyl)-4-fluoro-1-oxo-isoindolin-5-yl]piperidine-4-carbaldehyde (31 mg, 0.08 mmol, 1 eq) and 4-methylmorpholine (26 mg, 0.25 mmol, 0.03 mL, 3 eq) in N,N-dimethylformamide (2 mL) was added 3-[6-[4-[2-(4-fluoro-4-piperidyl)ethyl]piperazin-1-yl]pyrimidin-4-yl]-5-(1-methylcyclopropoxy)-1H-indazole (50 mg, 0.08 mmol, 1 eq, trifluoroacetate) was stirred for 0.5 h, sodium triacetoxyborohydride (36 mg, 0.17 mmol, 2 eq) was added to the mixture, the reaction mixture was stirred at 20° C. for 11.5 h. LCMS (EW7574-1671-P1B) showed the reaction was completed. The reaction mixture was quenched by aqueous water (10 mL) at 20° C., and extracted with ethyl acetate (20 mL×3), the combined organic layers were washed with brine (30 mL×3), dried over anhydrous sodium sulfate, filtered and concentrated under reduced pressure to give a residue. The residue was purified by semi-preparative reverse phase HPLC (column: Phenomenex luna C18 150*25 mm*10 um; mobile phase: [water(FA)-ACN]; B %: 8%-38%, 10 min). Compound 3-[4-fluoro-5-[4-[[4-fluoro-4-[2-[4-[6-[5-(1-methylcyclopropoxy)-1H-indazol-3-yl]pyrimidin-4-yl]piperazin-1-yl]ethyl]-1-piperidyl]methyl]-1-piperidyl]-1-oxo-isoindolin-2-yl]piperidine-2,6-dione (35.7 mg, 0.04 mmol, 48% yield, 95% purity, formate) was isolated as a colorless solid.

Exemplary Synthesis of Exemplary Compound 7: Compound 7 was prepared in a manner analogous to general schemes 2, 3, and 4 utilizing benzyl (3s,4r)-3-fluoro-4-formyl-piperidine-1-carboxylate.

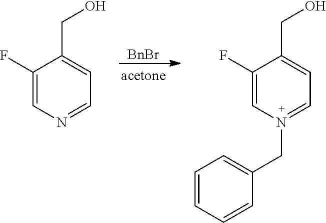

### Step 1:

To a solution of (3-fluoro-4-pyridyl)methanol (14.8 g, 116.43 mmol, 1 eq) in ACETONE (130 mL) was added BnBr (21.90 g, 128.07 mmol, 15.21 mL, 1.1 eq). The mixture was stirred at 65° C. for 16 hr. TLC (Petroleum ether:Ethyl acetate=0:1, Rf=0.1) showed a new spot. The reaction mixture was cooled to room temperature and diluted with MTBE (100 ml). The suspension was filtered, and the wet cake was then stirred with 25% acetone/MTBE v/v (200 ml) and filtered. The filter mass was then dried in vacuo to afford (1-benzyl-3-fluoro-pyridin-1-ium-4-yl)methanol (25 g, 114.55 mmol, 98.39% yield) as a yellow solid.

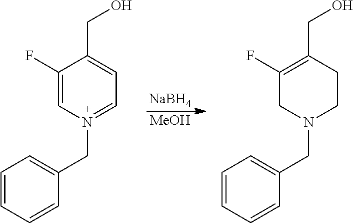

### Step 2

To a solution of (1-benzyl-3-fluoro-pyridin-1-ium-4-yl)methanol (25 g, 114.55 mmol, 1 eq) in MeOH (300 mL) at 0° C. and then was added NaBH₄ (6.50 g, 171.82 mmol, 1.5 eq) at 0° C. The mixture was stirred at 0° C. for 2 hr. TLC (Petroleum ether:Ethyl acetate=0:1, Rf=0.6) showed the reaction new spot. The reaction mixture was quenched with 100 mL of NH₄Cl (sat.). The aqueous phase was extracted with ethyl acetate (3×100 mL). The combined organic layers were washed with brine (3×80 mL), dried over anhydrous sodium sulfate, and concentrated under reduced pressure. The residue was purified by flash silica gel chromatography (ISCO®; 330 g SepaFlash® Silica Flash Column, Eluent of 0-100% Ethyl acetate/Petroleum ether gradient @ 80 mL/min) to afford (1-benzyl-5-fluoro-3,6-dihydro-2H-pyridin-4-yl)methanol (13 g, 58.75 mmol, 51.29% yield) as a yellow gum.

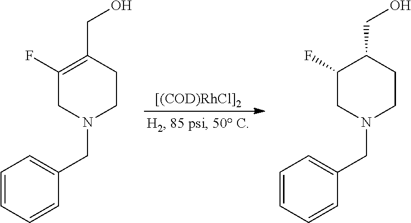

### Step 3

Under N₂ atmosphere, a three-necked round-bottom flask was charged with cyclopentane;dicyclohexyl-[(1R)-1-[2-(2-diphenylphosphanylphenyl)cyclopentyl]ethyl]phosphane;iron (22.5 mg, 33.55 umol, 1.48e-3 eq) chlororhodium;(1Z,5Z)-cycloocta-1,5-diene (7.5 mg, 15.21 umol, 6.73e-4 eq) and dry nitrogen degassed DCM (10 mL). The solution was stirred at ambient temperature for 45 min. A solution of (1-benzyl-5-fluoro-3,6-dihydro-2H-pyridin-4-yl)methanol (5 g, 22.60 mmol, 1 eq) in dry MeOH (50 mL) was added to a nitrogen purged 250 mL stainless steel pressure vessel. Subsequently, the aged catalyst solution from above was added into the vessel under nitrogen flow. The resulting mixture was degassed three times with H₂ and then (2.0 MPa, ca. 300 psi) heated at 50° C. under 2.0 MPa of H₂ for 36 hr with stirring. TLC (Petroleum ether:Ethyl acetate=0:1, Rf=0.3) showed the reaction new spot. The reaction vessel allowed to cool to room temperature and was purged with nitrogen. The reaction mixture was concentrated in vacuo to yield a dark brown oil. This concentrate was taken up in EtOAc (50 mL) and saturated aqueous NaHCO₃ (50 mL) was added. The mixture was stirred at room temperature for 30 min and the organic phase was separated. The aqueous phase was extracted with three times with EtOAc (60 ml). The combined organic phases were washed with brine, dried over Na₂SO₄, and concentrated under vacuum. The residue was purified by flash silica gel chromatography (ISCO®; 330 g SepaFlash® Silica Flash Column, Eluent of 0-100% Ethylacetate/Petroleum ether gradient @ 60 mL/min) to afford (1-benzyl-3-fluoro-4-piperidyl)methanol (2.95 g, 13.21 mmol, 58.47% yield) or the enantiomer thereof as yellow gum.

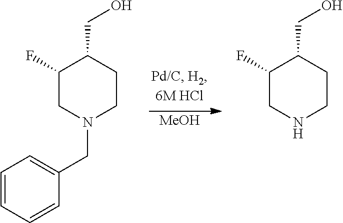 [SMILES: "MeOH" ; OC[C@@H]1CCN(Cc2ccccc2)C[C@@H]1F ; OC[C@@H]1CCNC[C@@H]1F]

### Step 4

To a stirred solution of ((3r,4s)-1-benzyl-3-fluoropiperidin-4-yl)methanol (2.95 g, 13.21 mmol, 1 eq) or the enantiomer thereof in MeOH (10 mL) was added Pd/C (300 mg, 13.21 mmol, 10% purity, 1 eq) and HCl (6 M, 3 mL, 1.36 eq). The mixture was purged with hydrogen three times at 15 psi. After stirring at 50° C. for 6 hours, the starting material was consumed. The reaction mixture then was filtered through celite, the filter pad rinsed with MeOH, and the filtrate concentrated in vacuo to afford [(3s,4r)-3-fluoro-4-piperidyl]methanol (2.2 g, crude, HCl) or the enantiomer thereof as a white solid.

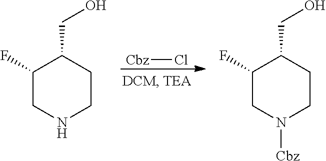

### Step 5

To a solution of [(3s,4r)-3-fluoro-4-piperidyl]methanol (2.2 g, 12.97 mmol, 1.18 eq, HCl) or the enantiomer thereof in DCM (50 mL) was added TEA (5.56 g, 54.96 mmol, 7.65 mL, 5 eq) at 0° C. and stirred at 0° C. for 0.5 h. Then CbzCl (3.60 g, 21.10 mmol, 3 mL, 1.92 eq) was added and stirred at 0° C. for 2 h under N₂. LCMS(EB13-1242-P1A) showed desired product. TLC (Petroleum ether:Ethyl acetate=0:1, Rf=0.5) showed the reaction a new spot. The reaction diluted with water (40 mL). The mixture was extracted with ethyl acetate (50 mL*3). dried over anhydrous Na₂SO₄, concentrated in vacuum to give a residue. The residue was purified by silica gel column chromatography (0-100% Ethyl acetate in Petroleum ether) to give benzyl (3s,4r)-3-fluoro-4-(hydroxymethyl)piperidine-1-carboxylate (2.8 g, 10.48 mmol, 95.30% yield) or the enantiomer thereof as a colorless gum.

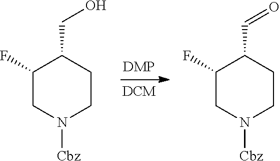 [SMILES: O=C(OCc1ccccc1)N1CC[C@@H](CO)[C@@H](F)C1 ; O=C[C@@H]1CCN(C(=O)OCc2ccccc2)C[C@@H]1F]

### Step 6

To a solution of benzyl (3s,4r)-3-fluoro-4-(hydroxymethyl)piperidine-1-carboxylate (500 mg, 1.87 mmol, 1 eq) or the enantiomer thereof in DCM (10 mL) at 20° C. Then DMP (1000.00 mg, 2.36 mmol, 729.93 uL, 1.26 eq) was added and stirred at 20° C. for 1 h. LCMS (EB13-1244-P1A1) showed desired product. TLC (Petroleum ether:Ethyl acetate=0:1, Rf=0.6) showed the reaction a new spot. The reaction mixture was quenched by addition Sat.NaHCO₃ adjust pH˜8 at 0° C., and extracted with DCM (20 mL*3). The combined organic layers were washed with Sat.NaSO₃ (20 mL*2) and brine (10 mL*2), dried over Na₂SO₄, filtered and concentrated under reduced pressure to give benzyl (3s,4r)-3-fluoro-4-formyl-piperidine-1-carboxylate (450 mg, crude) or the enantiomer thereof as a colorless gum.

Exemplary Synthesis of Exemplary Compound 8: Compound 8 was prepared in a manner analogous to compound 6.

Exemplary Synthesis of Exemplary Compound 9: Compound 9 was prepared in a manner analogous to compound 7.

Exemplary Synthesis of Exemplary Compound 10: Compound 10 was prepared in a manner analogous to compound 4 utilizing intermediate benzyl 4-[[(2R,5R)-5-(hydroxymethyl)tetrahydropyran-2-yl]methyl]piperazine-1-carboxylate or the enantiomer thereof.

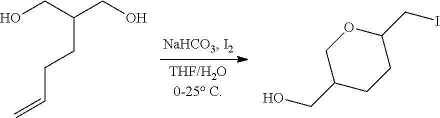

### Step 1

To a stirred suspension of iodine (122.83 g, 483.93 mmol, 97.48 mL, 1.5 eq) and NaHCO₃ (40.65 g, 483.93 mmol, 18.82 mL, 1.5 eq) in tetrahydrofuran (200 mL) and water (80 mL) was added the solution of 2-but-3-enylpropane-1,3-diol (42 g, 322.62 mmol, 1 eq) in THF (200 mL) at 0° C. The mixture was stirred at 25° C. for 12 hours. LCMS showed the desired mass was detected. Saturated Na2S2O3 aqueous (500 mL) was added to the mixture slowly at 0° C. and the mixture was stirred at 25° C. for 10 minutes. The solution was extracted with EtOAc (300 ml*3). The combined organic layer was dried over anhydrous sodium sulfate, filtered, and the filtrate was concentrated under vacuum. The residue was purified by silica gel chromatography (PE:EtOAc=100:1-1:1). [6-(iodomethyl)tetrahydropyran-3-yl]methanol (34 g, 132.77 mmol, 41.15% yield) was obtained as a light yellow oil.

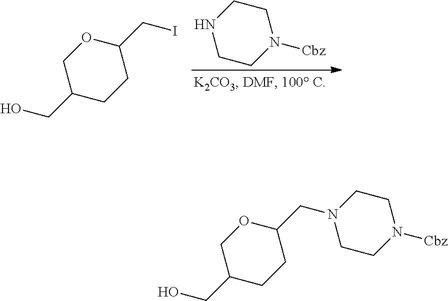

### Step 2

To a stirred solution of [6-(iodomethyl)tetrahydropyran-3-yl]methanol (12.4 g, 48.42 mmol, 1 eq) in DMF (100 mL) was added K2CO3 (16.73 g, 121.06 mmol, 2.5 eq) and benzyl piperazine-1-carboxylate (12.80 g, 58.11 mmol, 11.23 mL, 1.2 eq). The reaction mixture was stirred at 100° C. for 12 hours. LCMS showed desired mass was detected. Water (300 mL) was added to the mixture and the aqueous was extracted with ethyl acetate (200 mL*3). The combined organic layer was washed with brine (200 mL*2), dried over anhydrous Na2SO4, filtered and the filtrate was concentrated under vacuum. The residue was purified by silica gel chromatography (DCM:MeOH=100:1-10:1). Benzyl 4-[[5-hydroxymethyl)tetrahydropyran-2-yl]methyl]piperazine-1-carboxylate (13 g, 37.31 mmol, 77.05% yield) was obtained as a mixture of diasteromers as a colorless oil.

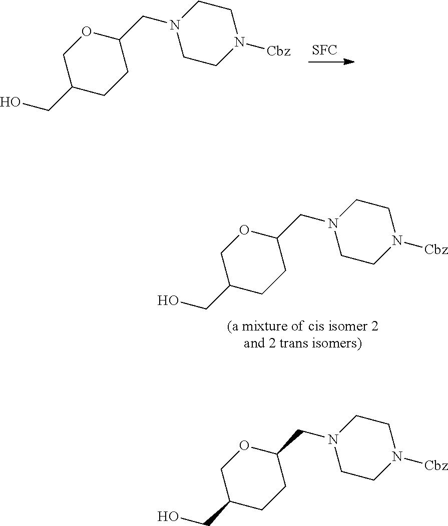

### Step 3

The cis isomer benzyl 4-(((2R,5S)-5-(hydroxymethyl)tetrahydro-2H-pyran-2-yl)methyl)piperazine-1-carboxylate or the enantiomer thereof (1.53 g, 3.92 mmol, 11.01% yield, 89.24% purity, peak 4: de=100%, Rt=1.416 min) was obtained by SCF purification of the diastereomeric mixture of benzyl 4-[[5-(hydroxymethyl)tetrahydropyran-2-yl]methyl]piperazine-1-carboxylate (12.40 g, 35.59 mmol, 1 eq) by SFC. The first fraction obtained (10.19 g, 29.25 mmol, 82.19% yield) was a mixture of a single cis isomer (peak 2: Rt=1.161 min) and trans isomers (peak 1: Rt=1.125 min, peak 3: Rt=1.195 min)

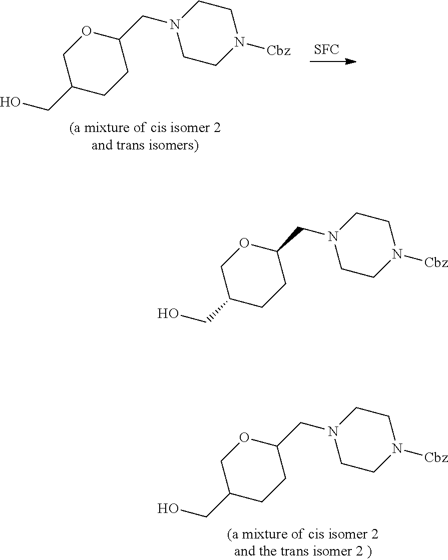

### Step 4:

Benzyl 4-[[(2R,5R)-5-(hydroxymethyl)tetrahydropyran-2-yl]methyl]piperazine-1-carboxylate or the enantiomer thereof (trans isomer confirmed by HSQC, peak 3: de %=100, Rt=2.614 min, 3.24 g, 8.86 mmol, 30.31% yield, 95.32% purity) was obtained as a light yellow oil by SFC resolution of the diastereomeric mixture of 1 cis isomer and 2 trans isomers (10.19 g, 29.25 mmol). The remaining diasteromers were obtained as a mixture (mixture of cis isomer 2 and trans isomer 2, 4.19 g, 12.03 mmol, 41.14% yield, peak 1: Rt=1.156 min, peak 2: Rt=1.244 min) as a light yellow oil.

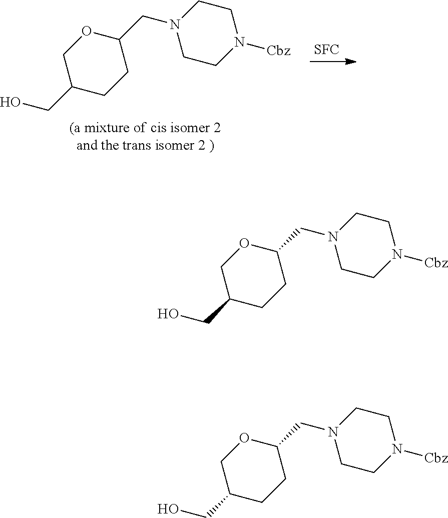

### Step 5:

The trans product 4-[[(2S,5S)-5-(hydroxymethyl)tetrahydropyran-2-yl]methyl]piperazine-1-carboxylate or the enantiomer thereof (2.41 g, 6.58 mmol, 54.71% yield, 95.11% purity, yellow oil, trans isomer 2, peak 1: de=100%, Rt=1.977 min) and cis product benzyl 4-[[(2S,5R)-5-(hydroxymethyl)tetrahydropyran-2-yl]methyl]piperazine-1-carboxylate or the enantiomer thereof (3.10 g, 8.45 mmol, 70.27% yield, 94.98% purity, yellow oil, cis isomer 2, peak 2: de=100%, Rt=2.088 min) were obtained by SFC separation of the cis and trans diasteromers.

Exemplary Synthesis of Exemplary Compound 11: Compound 11 was prepared in a manner analogous to compound 6 utilizing intermediate tert-butyl (4R)-4-[(1-benzyloxycarbonyl-4-piperidyl)oxy]-2,2-dimethyl-piperidine-1-carboxylate or the enantiomer thereof.

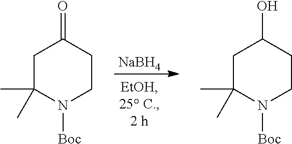 [SMILES: CC(C)(C)OC(=O)N1CCC(=O)CC1(C)C ; "EtOH,25° C., 2 h" ; "NaBH4" ; CC(C)(C)OC(=O)N1CCC(O)CC1(C)C]

### Step 1:

To a mixture of tert-butyl 2,2-dimethyl-4-oxo-piperidine-1-carboxylate (10 g, 43.99 mmol, 1 eq) in ethanol (100 mL) was added sodium borohydride (9.18 g, 242.62 mmol, 5.51 eq) in small portions at 0° C. The mixture was stirred at 25° C. for 2 hours. TLC showed the reaction was completed. A saturated aqueous solution of ammonium chloride is added. The ethanol is removed under reduced pressure. Then extracted with ethyl acetate (20 mL×3). The combined organic phase was washed with brine (30 mL), dried with anhydrous sodium sulfate, filtered and concentrated in vacuum. The crude product tert-butyl 4-hydroxy-2,2-dimethyl-piperidine-1-carboxylate (10 g, crude) was obtained as a colorless oil.

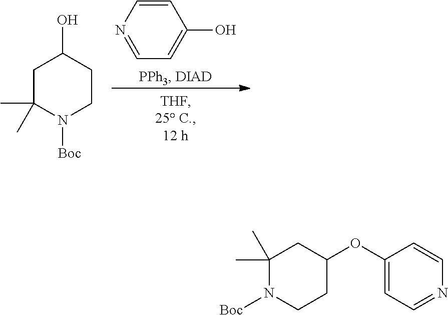

### Step 2:

To a mixture of pyridin-4-ol (4.98 g, 52.33 mmol, 1.2 eq) and tert-butyl 4-hydroxy-2,2-dimethyl-piperidine-1-carboxylate (10 g, 43.61 mmol, 1 eq) in tetrahydrofuran (100 mL) was added triphenylphosphane (17.16 g, 65.41 mmol, 1.5 eq) and diisopropylazodicarboxylate (13.23 g, 65.41 mmol, 12.7 mL, 1.5 eq) at 0° C. The mixture was stirred at 25° C. for 12 hours. LCMS showed the desired m/z was detected. The mixture was concentrated in reduced pressure at 40° C. The residue was diluted with Petroleum ether/Ethyl acetate (v/v=3/1) (60 mL) and filtered. The filtrate was concentrated in vacuum. The residue was purified by column chromatography (Petroleum ether/Ethyl acetate=10/1 to 3/1 to 1/1). The crude product tert-butyl 2,2-dimethyl-4-(4-pyridyloxy)piperidine-1-carboxylate (6.6 g, crude) was obtained as a colorless oil

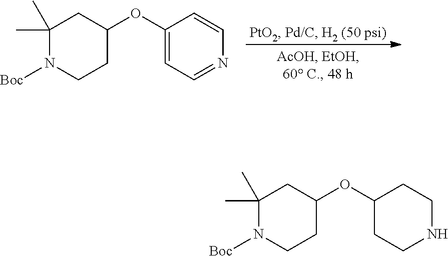 [SMILES: CC(C)(C)OC(=O)N1CCC(Oc2ccncc2)CC1(C)C ; "AcOH, EtOH,60° C., 48 h" ; "PtO2, Pd/C, H2 (50 psi)" ; CC(C)(C)OC(=O)N1CCC(OC2CCNCC2)CC1(C)C]

### Step 3:

To a solution of tert-butyl 2,2-dimethyl-4-(4-pyridyloxy)piperidine-1-carboxylate (6.00 g, 19.6 mmol, 1 eq) in acetic acid (20 mL) was added platinum oxide (1 g, 4.40 mmol) and Pd/C (1 g, 10% purity) under nitrogen atmosphere. The suspension was degassed under vacuum and purged with H₂ several times. The mixture was stirred under H₂ (50 psi) at 60° C. for 48 hours. LCMS showed the starting material was consumed completely. The reaction mixture was filtered, and the filter was concentrated. The residue was dissolved into water (50 mL) and neutralized by 1N sodium hydroxide solution until pH-9. The aqueous phase was extracted with ethyl acetate (30 mL×3), the combined organic phase was washed with brine (20 mL), dried with anhydrous sodium sulfate, filtered, and concentrated in vacuum. The crude product tert-butyl 2,2-dimethyl-4-(4-piperidyloxy)piperidine-1-carboxylate (5.9 g, crude) was obtained as a colorless oil.

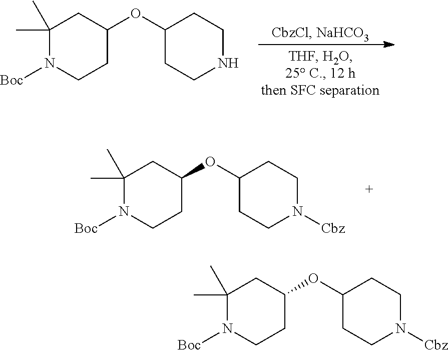

### Step 4:

To a mixture of tert-butyl 2,2-dimethyl-4-(4-piperidyloxy)piperidine-1-carboxylate (5.2 g, 16.64 mmol, 1 eq) in tetrahydrofuran (100 mL) and water (50 mL) was added benzyl chloroformate (4.26 g, 24.9 mmol, 3.6 mL, 1.5 eq) and sodium hydrogen carbonate (2.80 g, 33.3 mmol, 2 eq) at 0° C. The mixture was stirred at 25° C. for 12 hours. LCMS showed the reaction was completed. The mixture was diluted with water (50 mL) and extracted with ethyl acetate (50 mL×3). The combined organic phase was washed with brine (50 mL), dried with anhydrous sodium sulfate, filtered, and concentrated under vacuum. The residue was purified by column chromatography (Petroleum ether/Ethyl acetate=20/1 to 10/1). The residue was purified by prep-HPLC (column: Phenomenex luna C18 (250*70 mm, 10 um); mobile phase: [water(FA)-ACN]; B %: 70%-100%, 21 min). The residue was further separated by SFC (column: DAICEL CHIRALPAK AD (250 mm*30 mm, 10 um); mobile phase: [0.1% NH3H2O MEOH]; B %: 45%-45%, 5.2 min). Compound tert-butyl (4S)-4-[(1-benzyloxycarbonyl-4-piperidyl)oxy]-2,2-dimethyl-piperidine-1-carboxylate or the enantiomer thereof (1.25 g, 2.80 mmol, 22.32% yield) was obtained as a colorless oil (Rt=1.3 min). Compound tert-butyl (4R)-4-[(1-benzyloxycarbonyl-4-piperidyl)oxy]-2,2-dimethyl-piperidine-1-carboxylate or the enantiomer thereof (1.25 g, 2.80 mmol, 22.32% yield) was obtained as a colorless oil (Rt=2.2 min).

### Exemplary Synthesis of Exemplary Compound 12:

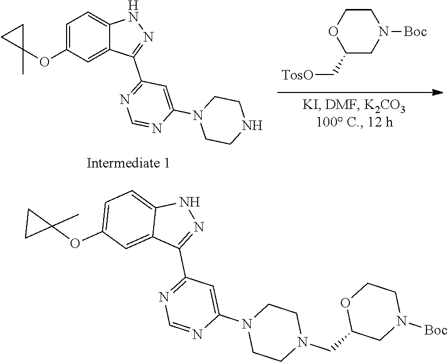

### Step 1

To a solution of 5-(1-methylcyclopropoxy)-3-(6-piperazin-1-ylpyrimidin-4-yl)-1H-indazole (Intermediate 1) (2 g, 5.17 mmol, 1 eq, HCl) and tert-butyl (2R)-2-(p-tolylsulfonyloxymethyl)morpholine-4-carboxylate (2.88 g, 7.75 mmol, 1.5 eq) in N,N-dimethylformamide (10 mL) was added potassium iodide (858 mg, 5.17 mmol, 1 eq) and potassium carbonate (2.86 g, 20.68 mmol, 4 eq). The mixture was stirred at 100° C. for 12 h. LCMS showed reactant was consumed completely and one main peak with desired was detected. Water (200 mL) was added and the mixture was extracted with ethyl acetate (100 mL×2). The combined organic layer was washed with brine (150 mL×2), dried over anhydrous sodium sulfate, filtered, and concentrated under reduced pressure to give a residue. The residue was purified by column chromatography (dichloromethane:Methanol=10:1) to afford tert-butyl (2S)-2-[[4-[6-[5-(1-methylcyclopropoxy)-1H-indazol-3-yl]pyrimidin-4-yl]piperazin-1-yl]methyl]morpholine-4-carboxylate (990 mg, 1.80 mmol, 34% yield) was obtained as a white solid.

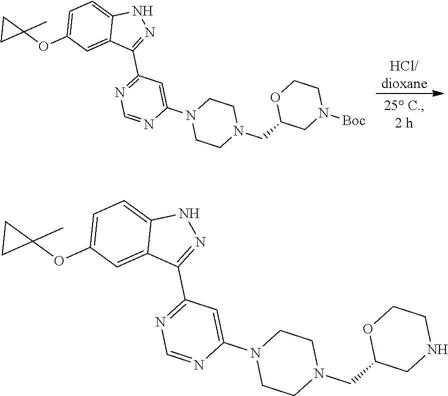 [SMILES: CC(C)(C)OC(=O)N1CCO[C@@H](CN2CCN(c3cc(-c4n[nH]c5ccc(OC6(C)CC6)cc45)ncn3)CC2)C1 ; "25° C., 2 h" ; "HCl/dioxane" ; CC1(Oc2ccc3[nH]nc(-c4cc(N5CCN(C[C@H]6CNCCO6)CC5)ncn4)c3c2)CC1]

### Step 2:

A solution of HCl/dioxane (4 M, 54.58 uL, 4 mL) was added to tert-butyl (2S)-2-[[4-[6-[5-(1-methylcyclopropoxy)-1H-indazol-3-yl]pyrimidin-4-yl]piperazin-1-yl]methyl]morpholine-4-carboxylate Intermediate (120 mg, 218.32 umol, 1 eq). The mixture was stirred at 25° C. for 2 h. The mixture was concentrated under vacuum to obtain (2R)-2-[[4-[6-[5-(1-methylcyclopropoxy)-1H-indazol-3-yl]pyrimidin-4-yl]piperazin-1-yl]methyl]morpholine (106 mg, 218.31 umol, 98% yield, HCl) as a yellow solid.

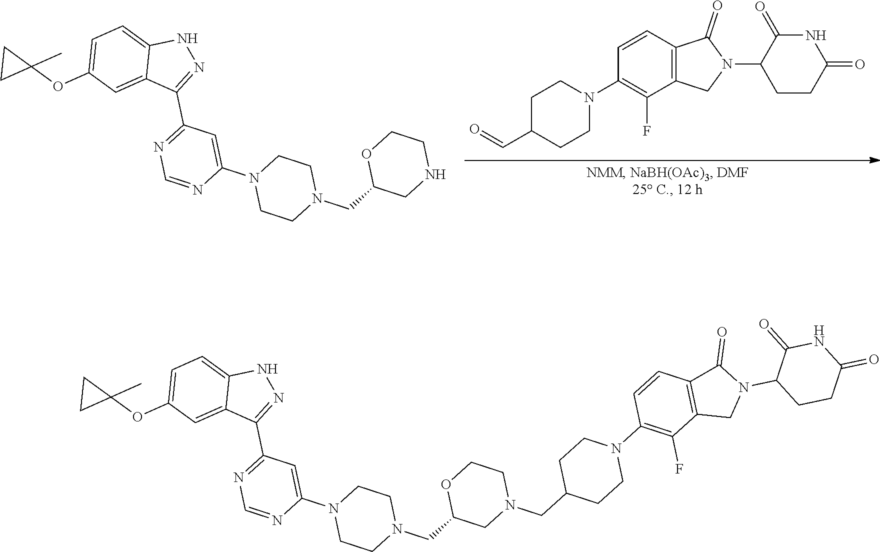 [SMILES: CC1(Oc2ccc3[nH]nc(-c4cc(N5CCN(C[C@H]6CNCCO6)CC5)ncn4)c3c2)CC1 ; O=CC1CCN(c2ccc3c(c2F)CN(C2CCC(=O)NC2=O)C3=O)CC1 ; "NMM, NaBH(OAc)3, DMF25° C., 12 h" ; CC1(Oc2ccc3[nH]nc(-c4cc(N5CCN(C[C@H]6CN(CC7CCN(c8ccc9c(c8F)CN(C8CCC(=O)NC8=O)C9=O)CC7)CCO6)CC5)ncn4)c3c2)CC1]

### Step 3:

To a solution of (2R)-2-[[4-[6-[5-(1-methylcyclopropoxy)-1H-indazol-3-yl]pyrimidin-4-yl]piperazin-1-yl]methyl]morpholine (106 mg, 218.10 umol, 1 eq, HCl) and 1-[2-(2,6-dioxo-3-piperidyl)-4-fluoro-1-oxo-isoindolin-5-yl]piperidine-4-carbaldehyde (81.43 mg, 218.10 umol, 1 eq) in N,N-dimethylformamide (5 mL) was added n-methylmorpholine (44 mg, 436.21 umol, 47.96 uL, 2 eq) and Sodium borohydride acetate (92 mg, 436.21 umol, 2 eq). The mixture was stirred at 25° C. for 12 h. LCMS showed reactant was consumed completely and one main peak with desired MS was detected. The mixture was concentrated under vacuum. The residue was purified by prep-HPLC (0.1% NH₃·H₂O or 0.1% FA condition) to give 3-[4-fluoro-5-[4-[[(2S)-2-[[4-[6-[5-(1-methylcyclopropoxy)-1H-indazol-3-yl]pyrimidin-4-yl]piperazin-1-yl]methyl]morpholin-4-yl]methyl]-1-piperidyl]-1-oxo-isoindolin-2-yl]piperidine-2,6-dione (19.7 mg, 24.41 umol, 11% yield) as a white solid.

Exemplary Synthesis of Exemplary Compound 13: Compound 13 was prepared in a manner analogous to compounds 12 utilizing intermediate tertbutyl (2S)-2-(p-tolylsulfonyloxymethyl)morpholine-4-carboxylate.

### Exemplary Synthesis of Exemplary Compound 14:

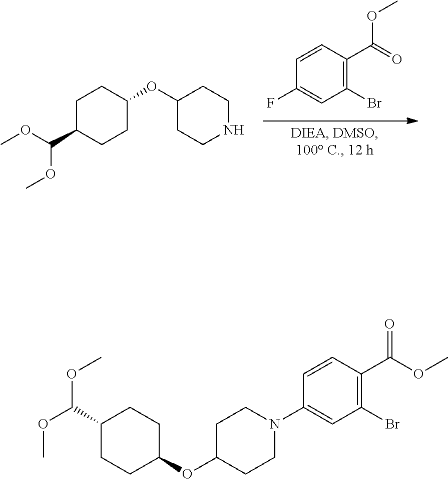

### Step 1:

To a solution of 4-[4-(dimethoxymethyl)cyclohexoxy]piperidine (2.43 g, 9.44 mmol, 1.1 eq) in dimethyl sulfoxide (20 mL) was added diisopropylethylamine (3.33 g, 25.75 mmol, 4.48 mL, 3 eq) and methyl 2-bromo-4-fluoro-benzoate (2 g, 8.58 mmol, 1.00 eq). The mixture was stirred at 100° C. for 12 h. Water (150 mL) was added to the mixture and aqueous was extracted with ethyl acetate (100 mL*2). The combined organic layers were washed with brine (80 mL*2), dried over anhydrous sodium sulfate, filtered, and concentrated under reduced pressure to give a residue. The residue was purified by flash silica gel chromatography (ISCO®; 40 g SepaFlash® Silica Flash Column, Eluent of 0-30% Ethyl acetate/Petroleum ether gradient @ 80 mL/min) to obtain methyl 2-bromo-4-[4-[4-(dimethoxymethyl)cyclohexoxy]-1-piperidyl]benzoate (2.9 g, 6.17 mmol, 72% yield) as a yellow oil.

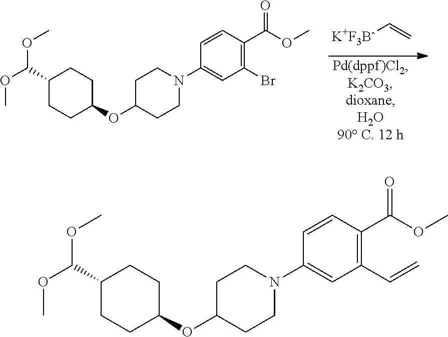

### Step 2:

A mixture of methyl 2-bromo-4-[4-[4-(dimethoxymethyl)cyclohexoxy]-1-piperidyl]benzoate (2.9 g, 6.17 mmol, 1 eq), potassium;trifluoro(vinyl)boranate (2.48 g, 18.50 mmol, 3 eq), [1,1-bis(diphenylphosphino)ferrocene]palladium,(ii)chloride (503 mg, 0.62 mmol, 0.1 eq), sodium carbonate (1.96 g, 18.50 mmol, 3 eq) in dioxane (30 mL) and water (3 mL) was degassed and purged with nitrogen for 3 times, and then the mixture was stirred at 90° C. for 12 h under nitrogen atmosphere. Water (150 ml) was added to the mixture and aqueous was extracted with ethyl acetate (100 mL*2). The combined organic layers were washed with brine (80 mL*20), dried over anhydrous sodium sulfate, filtered, and concentrated under reduced pressure to give a residue. The residue was purified by flash silica gel chromatography (ISCO®; 40 g SepaFlash® Silica Flash Column, Eluent of 0-30% Ethyl acetate/Petroleum ether gradient @ 80 mL/min). Methyl 4-[4-[4-(dimethoxymethyl)cyclohexoxy]-1-piperidyl]-2-vinyl-benzoate (2.27 g, 5.44 mmol, 88% yield) was obtained as a yellow oil.

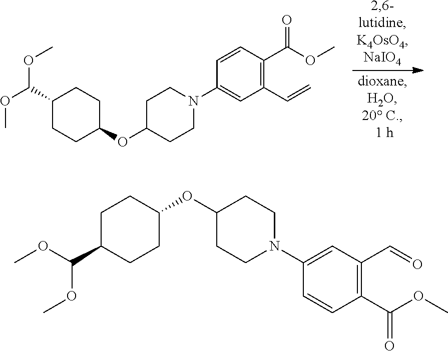

### Step 3

To a stirred solution of methyl 4-[4-[4-(dimethoxymethyl)cyclohexoxy]-1-piperidyl]-2-vinyl-benzoate (2.07 g, 4.96 mmol, 1 eq) in dioxane (60 mL) and water (20 mL) was added 2,6-lutidine (1.06 g, 9.91 mmol, 1.15 mL, 2 eq), dipotassium;dioxido(dioxo)osmium;dihydrate (36 mg, 0.099 mmol, 0.02 eq) and sodium periodate (4.24 g, 19.82 mmol, 1.10 mL, 4 eq) in portions. The reaction mixture was stirred at 20° C. for 1 h. Water (150 mL) was added to the mixture and aqueous was extracted with ethyl acetate (100 mL*2). The combined organic layers were washed with brine (80 mL*20), dried over anhydrous sodium sulfate, filtered, and concentrated under reduced pressure to give a residue. The residue was purified by flash silica gel chromatography (ISCO®; 40 g SepaFlash® Silica Flash Column, Eluent of 0-30% Ethyl acetate/Petroleum ether gradient @ 40 mL/min) to give methyl 4-[4-[4-(dimethoxymethyl)cyclohexoxy]-1-piperidyl]-2-formyl-benzoate (1.25 g, 2.97 mmol, 60% yield) as a yellow oil.

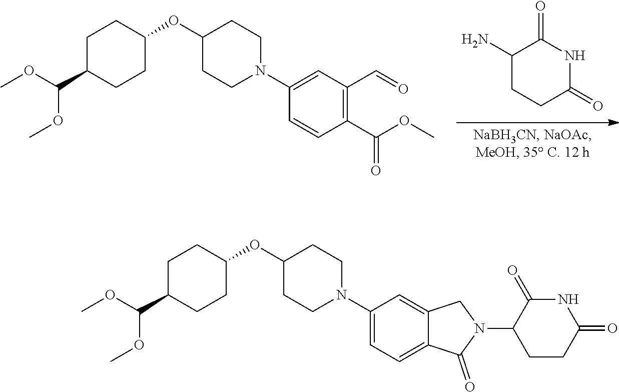

### Step 4:

To a stirred solution of methyl 4-[4-[4-(dimethoxymethyl)cyclohexoxy]-1-piperidyl]-2-formyl-benzoate (1.15 g, 2.74 mmol, 1 eq) and 3-aminopiperidine-2,6-dione (541 mg, 3.29 mmol, 1.2 eq, hydrochloride) in methanol (100 mL) was added sodium acetate (675 mg, 8.22 mmol, 3 eq). The mixture was stirred at 25° C. for 5 min. Then methyl 4-[4-[4-(dimethoxymethyl)cyclohexoxy]-1-piperidyl]-3-fluoro-2-formyl-benzoate (110 mg, 182.86 mmol, 1 eq) was added. The mixture was stirred at 25° C. for another 0.5 h. At last, sodium cyanoborohydride (345 mg, 5.48 mmol, 2 eq) was added. The resulting mixture was stirred at 35° C. for 12 h. The reaction mixture was concentrated under reduced pressure to give a residue. The residue was diluted with water (30 mL) and extracted with ethyl acetate (20 mL*2), dried over sodium sulfate, filtered, and concentrated under reduced pressure to give a residue. The residue was purified by prep-TLC (Dichloromethane:Methanol=12:1). Compound 3-[5-[4-[4-(dimethoxymethyl)cyclohexoxy]-1-piperidyl]-1-oxo-isoindolin-2-yl]piperidine-2,6-dione (1.1 g, 2.20 mmol, 80% yield) was obtained as a white solid.

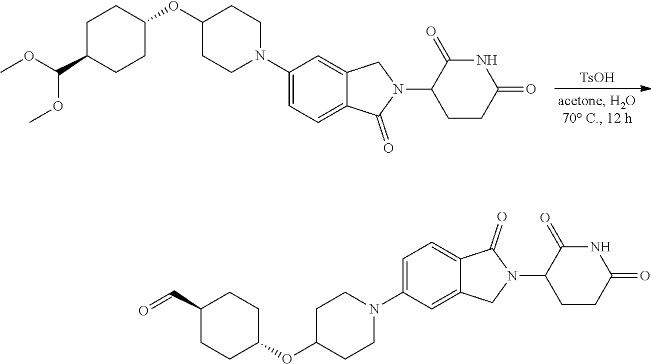 [SMILES: COC(OC)[C@H]1CC[C@H](OC2CCN(c3ccc4c(c3)CN(C3CCC(=O)NC3=O)C4=O)CC2)CC1 ; "TsOH" ; "acetone, H2O70° C., 12 h" ; O=C[C@H]1CC[C@H](OC2CCN(c3ccc4c(c3)CN(C3CCC(=O)NC3=O)C4=O)CC2)CC1]

### Step 5

To a stirred solution of 3-[5-[4-[4-(dimethoxymethyl)cyclohexoxy]-1-piperidyl]-1-oxo-isoindolin-2-yl]piperidine-2,6-dione (1.1 g, 2.20 mmol, 1 eq) in acetone (20 mL) and water (2 mL) was added 4-methylbenzenesulfonic acid (76 mg, 0.44 mmol, 0.2 eq) in portions. The reaction mixture was stirred at 70° C. for 12 h and cooled. The mixture was added ethyl acetate (30 ml), filtered, and concentrated under reduced pressure to give a residue. The crude product 4-[[1-[2-(2,6-dioxo-3-piperidyl)-1-oxo-isoindolin-5-yl]-4-piperidyl]oxy]cyclohexanecarbaldehyde (500 mg, 1.01 mmol, 46% yield, 91% purity) was obtained as a brown oil without further purification.

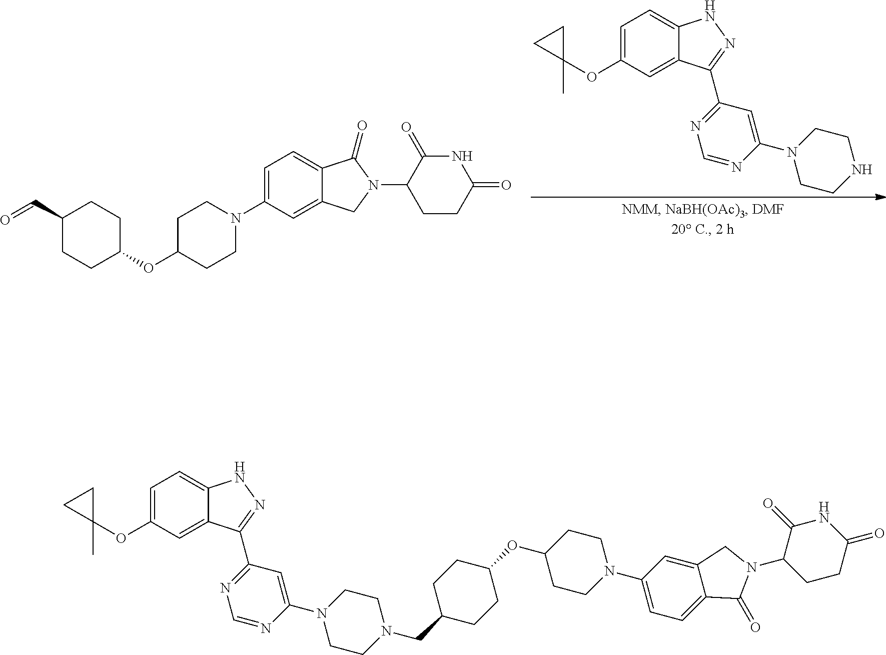 [SMILES: O=C[C@H]1CC[C@H](OC2CCN(c3ccc4c(c3)CN(C3CCC(=O)NC3=O)C4=O)CC2)CC1 ; CC1(Oc2ccc3[nH]nc(-c4cc(N5CCNCC5)ncn4)c3c2)CC1 ; CC1(Oc2ccc3[nH]nc(-c4cc(N5CCN(C[C@H]6CC[C@H](OC7CCN(c8ccc9c(c8)CN(C8CCC(=O)NC8=O)C9=O)CC7)CC6)CC5)ncn4)c3c2)CC1]

### Step 6

To a solution of 4-[[1-[2-(2,6-dioxo-3-piperidyl)-1-oxo-isoindolin-5-yl]-4-piperidyl]oxy]cyclohexanecarbaldehyde (150 mg, 0.33 mmol, 1 eq) and 5-(1-methylcyclopropoxy)-3-(6-piperazin-1-ylpyrimidin-4-yl)-1H-indazole (141 mg, 0.36 mmol, 1.1 eq, hydrochloride) in N,N-dimethylformamide (4 mL) was added 4-methylmorpholine (100 mg, 0.99 mmol, 3 eq) and sodium triacetoxyborohydride (140 mg, 0.66 mmol, 2 eq). The mixture was stirred at 20° C. for 2 h. The reaction mixture was concentrated under reduced pressure to give a residue, the residue was diluted with water (30 mL) and extracted with ethyl acetate (20 mL*2), dried over sodium sulfate, filtered, and concentrated under reduced pressure to give a residue. The mixture was purified by prep-HPLC (column: Unisil 3-100 C18 Ultra 150*50 mm*3 um; mobile phase: [water(FA)-ACN]; B %: 14%-44%, 7 min) to give 3-[5-[4-[4-[[4-[6-[5-(1-methylcyclopropoxy)-1H-indazol-3-yl]pyrimidin-4-yl]piperazin-1-yl]methyl]cyclohexoxy]-1-piperidyl]-1-oxo-isoindolin-2-yl]piperidine-2,6-dione (177.7 mg, 0.23 mmol, 68% yield) as white solid.

Exemplary Synthesis of Exemplary Compound 15: Compound 15 was prepared in a manner analogous to compound 6 using intermediate benzyl 4-[2,2-difluoro-2-(4-piperidyl)ethyl]piperazine-1-carboxylate.

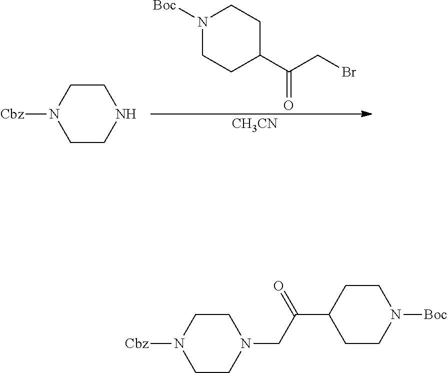 [SMILES: O=C(OCc1ccccc1)N1CCNCC1 ; CC(C)(C)OC(=O)N1CCC(C(=O)CBr)CC1 ; "CH3CN" ; CC(C)(C)OC(=O)N1CCC(C(=O)CN2CCN(C(=O)OCc3ccccc3)CC2)CC1]

### Step 1

To a solution of tert-butyl 4-(2-bromoacetyl)piperidine-1-carboxylate (695.05 mg, 2.27 mmol, 1 eq) in MeCN (10 mL) was stirred at 20° C. for 20 min. Then the mixture was added benzyl piperazine-1-carboxylate (500 mg, 2.27 mmol, 438.60 uL, 1 eq) and stirred at 20° C. for 16 hr under N₂. TLC (Dichloromethane:Methanol=10:1, Rf=0.6) showed no start material and a new spot. The residue was diluted with H₂O (30 mL) extracted with ethyl acetate (50 mL×3). The combined organic layers were washed with brine (45 mL), dried over anhydrous sodium sulfate, filtered and concentrated under reduced pressure to give a residue. The residue was purified by silica gel column chromatography (0 to 10% Dichloromethane in Methanol) to give benzyl 4-[2-(1-tert-butoxycarbonyl-4-piperidyl)-2-oxo-ethyl]piperazine-1-carboxylate (950 mg, 1.58 mmol, 69.51% yield, 74% purity) as a yellow gum.

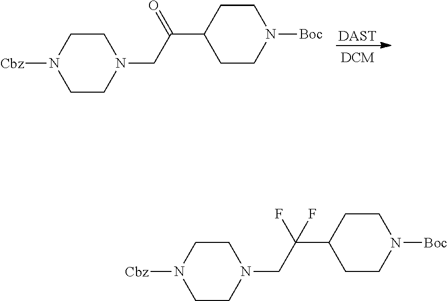

### Step 2

To a solution of benzyl 4-[2-(1-tert-butoxycarbonyl-4-piperidyl)-2-oxo-ethyl]piperazine-1-carboxylate (920 mg, 2.06 mmol, 1 eq) in DCM (30 mL) was stirred at 0° C. for 20 min. Then the mixture was added DAST (11.65 g, 72.27 mmol, 9.55 mL, 35 eq) and stirred at 20° C. for 2 hr under N₂. TLC (Dichloromethane:Methanol=10:1, Rf=0.5) showed no start material and a new spot. The reaction was cooled to 0° C. and quenched with aqueous NaHCO₃ (90 mL) extracted with ethyl acetate (50 mL×2). The combined organic layers were washed with brine (45 mL), dried over anhydrous sodium sulfate, filtered and concentrated under reduced pressure to give a residue. The residue was purified by silica gel column chromatography (0 to 10% Dichloromethane in Methanol) to give benzyl 4-[2-(1-tert-butoxycarbonyl-4-piperidyl)-2,2-difluoro-ethyl]piperazine-1-carboxylate (330 mg, 515.24 umol, 24.95% yield, 73% purity) as a yellow gum.

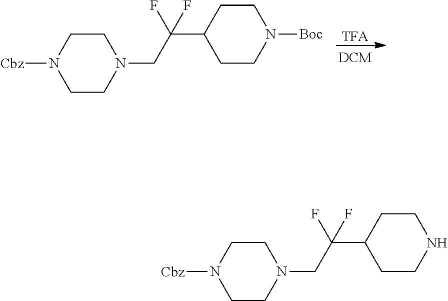 [SMILES: CC(C)(C)OC(=O)N1CCC(C(F)(F)CN2CCN(C(=O)OCc3ccccc3)CC2)CC1 ; O=C(OCc1ccccc1)N1CCN(CC(F)(F)C2CCNCC2)CC1 ; "TFA"]

### Step 3

To a solution of benzyl 4-[2-(1-tert-butoxycarbonyl-4-piperidyl)-2,2-difluoro-ethyl]piperazine-1-carboxylate (100 mg, 213.88 umol, 1 eq) in DCM (2 mL) was added TFA (2.31 g, 20.26 mmol, 1.5 mL, 94.72 eq) and stirred at 20° C. for 1 hr. TLC (Dichloromethane:Methanol=10:1, Rf=0.01) showed no start material and a new spot. The residue was concentrated under reduced pressure to give benzyl 4-[2,2-difluoro-2-(4-piperidyl)ethyl]piperazine-1-carboxylate (78 mg, crude) as a yellow gum.

Exemplary Synthesis of Exemplary Compound 16: Compound 16 was prepared in a manner analogous to compounds 1 and 2.

Exemplary Synthesis of Exemplary Compound 17: Compound 17 was prepared in a manner analogous to compound 12.

### Exemplary Synthesis of Exemplary Compound 18:

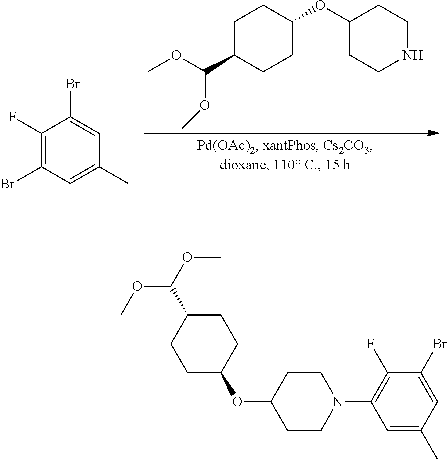

### Step 1

To a mixture of 4-[4-(dimethoxymethyl)cyclohexoxy]piperidine (3 g, 11.66 mmol, 1 eq), 1,3-dibromo-2-fluoro-5-methyl-benzene (3.12 g, 11.66 mmol, 1 eq), Cs₂CO₃ (5.70 g, 17.48 mmol, 1.5 eq) and Xantphos (404.68 mg, 699.39 umol, 0.06 eq) in dioxane (30 mL) was added Pd(OAc)₂ (104.68 mg, 466.26 umol, 0.04 eq) in one portion at 25° C. under N₂. The mixture was stirred at 100° C. for 12 hours and cooled. The mixture was poured into water (40 mL). The aqueous phase was extracted with ethyl acetate (40 mL*3). The combined organic phase was washed with brine (40 mL*3), dried with anhydrous Na₂SO₄, filtered, and concentrated under vacuum. The residue was purified by silica gel chromatography (Petroleum ether/Ethyl acetate=10/1) to afford 1-(3-bromo-2-fluoro-5-methyl-phenyl)-4-[4-(dimethoxymethyl)cyclohexoxy]piperidine (2.5 g, 5.63 mmol, 48.26% yield) as light yellow oil.

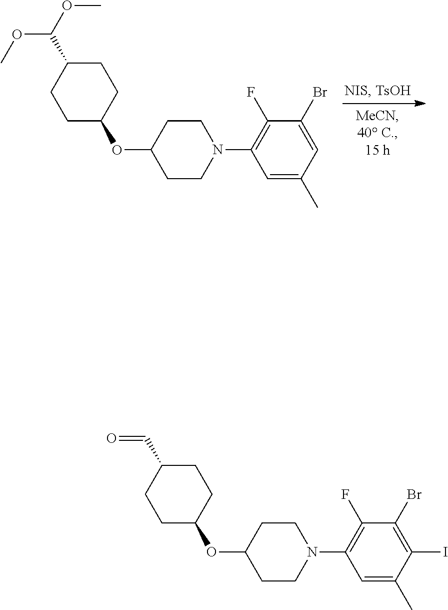

### Step 2

To a solution of 1-(3-bromo-2-fluoro-5-methyl-phenyl)-4-[4-(dimethoxymethyl)cyclohexoxy]piperidine (2.5 g, 5.63 mmol, 1 eq) and NIS (1.90 g, 8.44 mmol, 1.5 eq) in MeCN (15 mL) was added TsOH (968.77 mg, 5.63 mmol, 1 eq). The mixture was stirred at 40° C. for 12 hours. The mixture was poured into water (40 mL). The aqueous phase was extracted with ethyl acetate (40 mL*3). The combined organic phase was washed with brine (40 mL*3), dried with anhydrous Na₂SO₄, filtered, and concentrated under vacuum. The residue was purified by silica gel chromatography (Petroleum ether/Ethyl acetate=5:1) to afford 4-[[1-(3-bromo-2-fluoro-4-iodo-5-methyl-phenyl)-4-piperidyl]oxy]cyclohexanecarbaldehyde (0.45 g, 858.44 umol, 15.26% yield) as yellow oil.

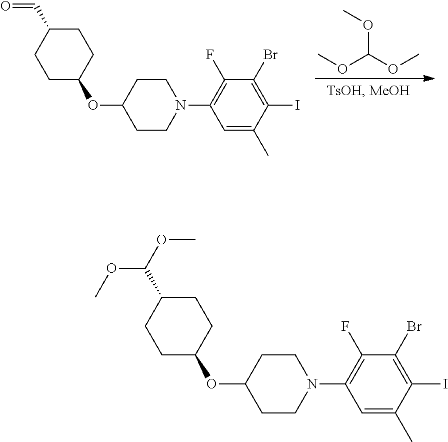

### Step 3:

To a solution of 4-[[1-(3-bromo-2-fluoro-4-iodo-5-methyl-phenyl)-4-piperidyl]oxy]cyclohexanecarbaldehyde (0.45 g, 858.44 umol, 1 eq) and trimethoxymethane (455.49 mg, 4.29 mmol, 470.55 uL, 5 eq) in MeOH (5 mL) was added TsOH (7.39 mg, 42.92 umol, 0.05 eq). The mixture was stirred at 25° C. for 1 hr. The mixture was poured into water (40 mL). The aqueous phase was extracted with ethyl acetate (40 mL*3). The combined organic phase was washed with brine (40 mL*3), dried with anhydrous Na₂SO₄, filtered, and concentrated under vacuum. The residue was purified by silica gel chromatography (Petroleum ether/Ethyl acetate=5/1) to afford 1-(3-bromo-2-fluoro-4-iodo-5-methyl-phenyl)-4-[4-(dimethoxymethyl)cyclohexoxy]piperidine (430 mg, 754.02 umol, 87.84% yield) as light yellow solid.

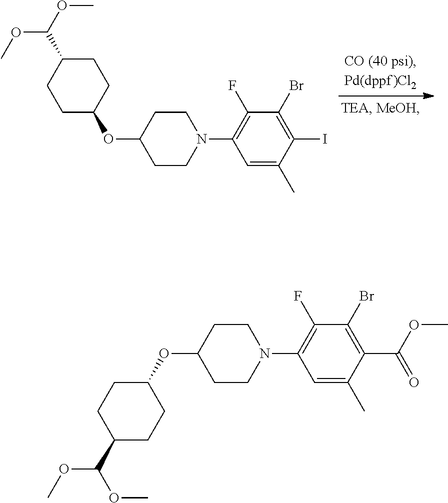

### Step 4:

To a mixture of 1-(3-bromo-2-fluoro-4-iodo-5-methyl-phenyl)-4-[4-(dimethoxymethyl)cyclohexoxy]piperidine (560 mg, 981.99 umol, 1 eq) and TEA (298.10 mg, 2.95 mmol, 410.04 uL, 3 eq) in MeOH (10 mL) was added Pd(dppf)Cl₂ (35.93 mg, 49.10 umol, 0.05 eq) in one portion at 25° C. The reaction was placed under CO at 40 Psi and stirred at 60° C. for 72 hours. The mixture was cooled to 25° C. and filtered. The residue was poured into water (15 mL) and extracted with ethyl acetate (25 mL*2). The combined organic phase was washed with brine (20 mL*2), dried with anhydrous Na₂SO₄, filtered, and concentrated under vacuum. The residue was purified by silica gel chromatography (Petroleum ether/Ethyl acetate=10/1) to afford methyl 2-bromo-4-[4-[4-(dimethoxymethyl)cyclohexoxy]-1-piperidyl]-3-fluoro-6-methyl-benzoate (340 mg, 676.73 umol, 68.91% yield) as light yellow oil.

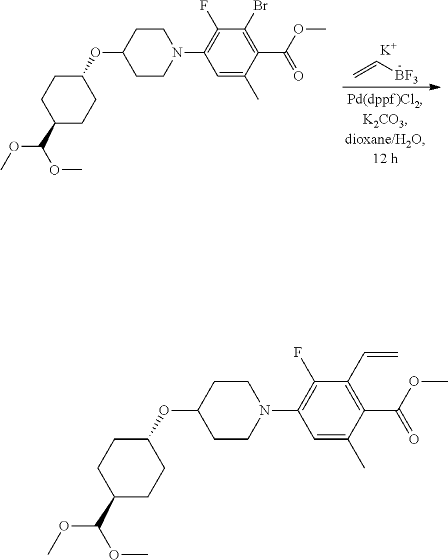

### Step 5:

To a solution of methyl 2-bromo-4-[4-[4-(dimethoxymethyl)cyclohexoxy]-1-piperidyl]-3-fluoro-6-methyl-benzoate (340 mg, 676.73 umol, 1 eq) in dioxane (5 mL) and H₂O (1 mL) was added Na₂CO₃ (179.32 mg, 1.69 mmol, 2.5 eq), potassium;trifluoro(vinyl)boranuide (271.95 mg, 2.03 mmol, 3 eq) and Pd(dppf)Cl₂·CH₂Cl₂ (38.69 mg, 47.37 umol, 0.07 eq). The mixture was stirred at 110° C. for 12 hours and cooled. The mixture was poured into water (30 mL) and the aqueous phase was extracted with ethyl acetate (30 mL*3). The combined organic phase was washed with brine (30 mL*2), dried with anhydrous Na₂SO₄, filtered, and concentrated under vacuum. The resulting residue was purified by silica gel chromatography (Petroleum ether:Ethyl acetate=5:1) to afford methyl 4-[4-[4-(dimethoxymethyl)cyclohexoxy]-1-piperidyl]-3-fluoro-6-methyl-2-vinyl-benzoate (270 mg, 600.60 umol, 88.75% yield) as yellow oil.

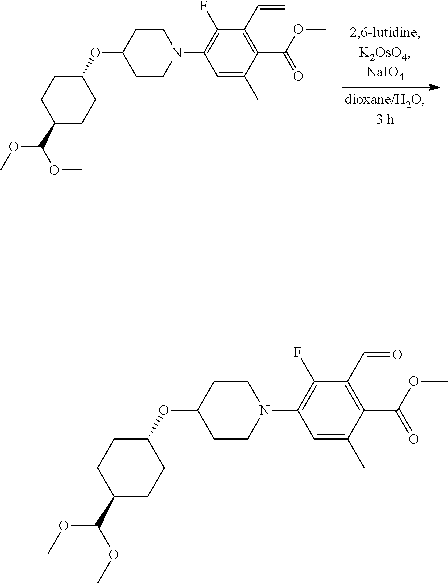

### Step 6:

To a solution of methyl 4-[4-[4-(dimethoxymethyl)cyclohexoxy]-1-piperidyl]-3-fluoro-6-methyl-2-vinyl-benzoate (270 mg, 600.60 umol, 1 eq) in dioxane (6 mL) and H₂O (2 mL) was added K₂OsO₄·2H₂O (4.43 mg, 12.01 umol, 0.02 eq), NaIO₄ (513.85 mg, 2.40 mmol, 133.12 uL, 4 eq) and 2,6-LUTIDINE (128.71 mg, 1.20 mmol, 139.90 uL, 2 eq) under a N₂ atmosphere. The mixture was stirred at 25° C. for 3 hours. The mixture was poured into water (30 mL) and stirred for 10 minutes. The aqueous phase was extracted with ethyl acetate (40 mL*3) and the combined organic phase was washed with brine (40 mL*3), dried with anhydrous Na₂SO₄, filtered, and concentrated under vacuum. The residue was purified by silica gel chromatography (Petroleum ether/Ethyl acetate=3/1) to afford methyl 4-[4-[4-(dimethoxymethyl)cyclohexoxy]-1-piperidyl]-3-fluoro-2-formyl-6-methyl-benzoate (180 mg, 398.65 umol, 66.38% yield) as yellow oil.

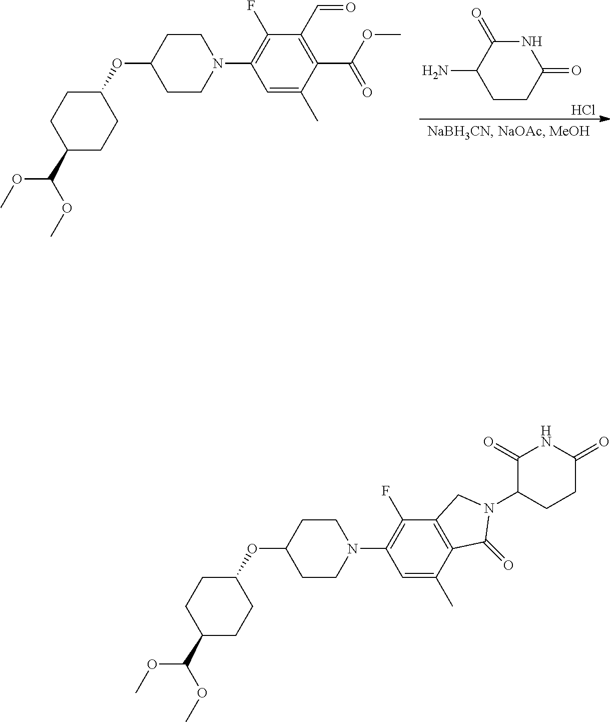

### Step 7:

To a mixture of 3-aminopiperidine-2,6-dione;hydrochloride (78.74 mg, 478.38 umol, 1.2 eq) in DCE (4 mL) and MeOH (1 mL) was added HOAc (2.39 mg, 39.86 umol, 2.28 uL, 0.1 eq), NaOAc (98.11 mg, 1.20 mmol, 3 eq) and methyl 4-[4-[4-(dimethoxymethyl)cyclohexoxy]-1-piperidyl]-3-fluoro-2-formyl-6-methyl-benzoate (180 mg, 398.65 umol, 1 eq) in one portion at 25° C. under N₂. The mixture was stirred at 25° C. for 0.5 h and NaBH₃CN (75.15 mg, 1.20 mmol, 3 eq) was added. The mixture was stirred for 11.5 h and Sat NaHCO₃ (aq) was then added (adjusted pH˜7). The aqueous phase was extracted with ethyl acetate (20 mL*3). The combined organic phase was washed with brine (20 mL*3), dried with anhydrous Na₂SO₄, filtered, and concentrated under vacuum to give a crude product. The crude product was stirred with TBME (2 ml) and sonicated for 3 minutes which afforded a precipitate that was filtered. The precipitate was washed with petroleum ether to afford 3-[5-[4-[4-(dimethoxymethyl)cyclohexoxy]-1-piperidyl]-4-fluoro-7-methyl-1-oxo-isoindolin-2-yl]piperidine-2,6-dione (230 mg, 389.38 umol, 97.68% yield, 90% purity) as white solid.

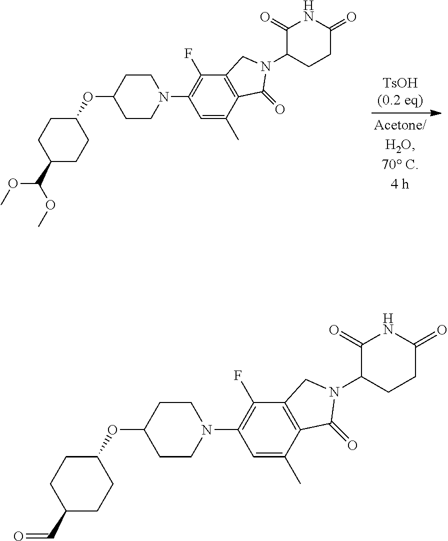 [SMILES: COC(OC)[C@H]1CC[C@H](OC2CCN(c3cc(C)c4c(c3F)CN(C3CCC(=O)NC3=O)C4=O)CC2)CC1 ; Cc1cc(N2CCC(O[C@H]3CC[C@H](C=O)CC3)CC2)c(F)c2c1C(=O)N(C1CCC(=O)NC1=O)C2]

### Step 8

To a solution of 3-[5-[4-[4-(dimethoxymethyl)cyclohexoxy]-1-piperidyl]-4-fluoro-7-methyl-1-oxo-isoindolin-2-yl]piperidine-2,6-dione (110 mg, 206.92 umol, 1 eq) in ACETONE (10 mL) and H₂O (1 mL) was added TsOH·H₂O (39.36 mg, 206.92 umol, 1 eq) and the mixture was stirred at 60° C. for 3 h under N₂ and then allowed to cool. The reaction mixture was poured into H₂O (10 mL) and basified with aqueous NaHCO₃ to pH=8. The mixture was extracted with ethyl acetate (10 mL*5), dried over anhydrous Na₂SO₄, filtered, and concentrated under vacuum to give a residue. The crude product was triturated with MeCN (10 mL) at 25° C. for 2 h. Then the solid was collected by filtration. The combined filtrates were concentrated to dryness to give 4-[[1-[2-(2,6-dioxo-3-piperidyl)-4-fluoro-7-methyl-1-oxo-isoindolin-5-yl]-4-piperidyl]oxy]cyclohexanecarbaldehyde (100 mg, 96.80 umol, 46.78% yield, 47% purity) as a yellow gum.

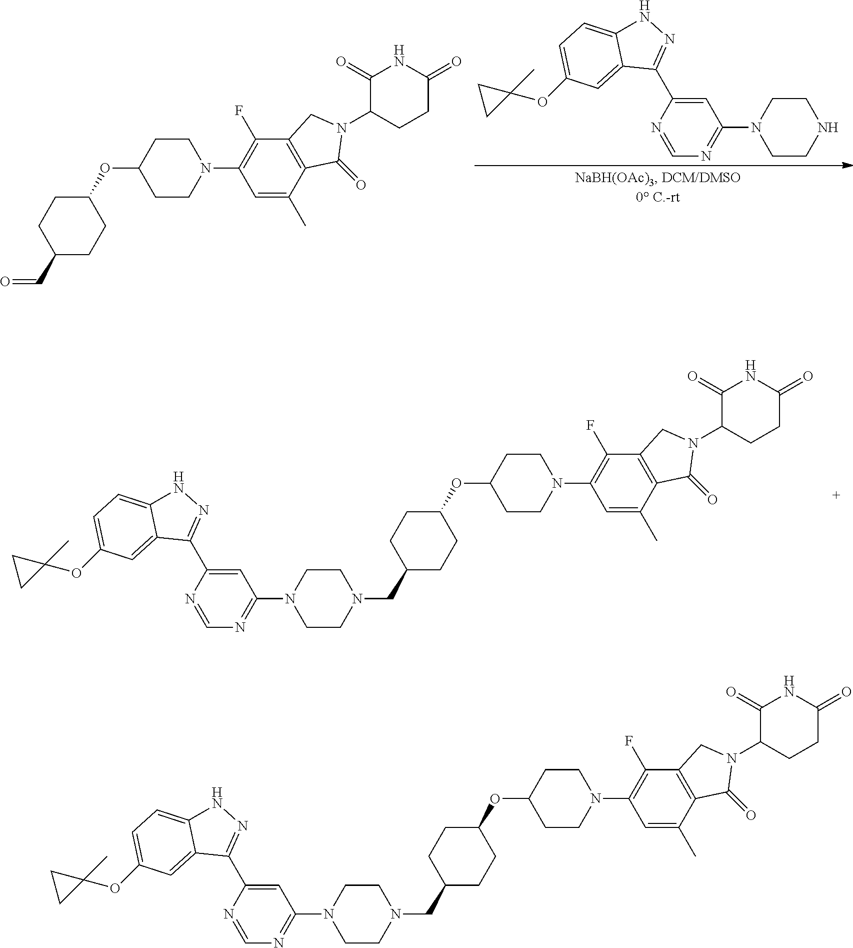

### Step 9

To a solution of 5-(1-methylcyclopropoxy)-3-(6-piperazin-1-ylpyrimidin-4-yl)-1H-indazole (70 MG, 199.76 umol, 1 eq) in DCM (10 mL) and DMSO (1 mL) was added 4-[[1-[2-(2,6-dioxo-3-piperidyl)-4-fluoro-7-methyl-1-oxo-isoindolin-5-yl]-4-piperidyl]oxy]cyclohexanecarbaldehyde (96.99 mg, 199.76 umol, 1 eq) at 0° C. and the mixture was stirred for 0.5 h. HOAc (3.60 mg, 59.93 umol, 3.43 uL, 0.3 eq) was added and stirring continued for 1 h. NaBH(OAc)₃ (84.68 mg, 399.52 umol, 2 eq) was added into the solution and the mixture was stirred for 2 h at 0° C. The mixture was then warmed to 25° C. for 12 h. The reaction mixture was quenched by addition water (10 mL) and extracted with dichloromethane (20 mL×3). The organic phase was washed with brine (10 mL), dried over anhydrous sodium sulfate, filtered, and concentrated under reduced pressure to a residue. The residue was purified by prep-HPLC (column: Phenomenex C18 75*30 mm*3 um; mobile phase: [water(FA)-ACN]; B %: 6%-46%, 26 min) to afford 3-(4-fluoro-7-methyl-5-(4-(((1r,4r)-4-((4-(6-(5-(1-methyl cyclopropoxy)-1H-indazol-3-yl)pyrimidin-4-yl)piperazin-1-yl)methyl)cyclohexyl)oxy)piperidin-1-yl)-1-oxoisoindolin-2-yl)piperidine-2,6-dione (30 mg, 35.16 umol, 17.60% yield, 96.11% purity) as white solid and 3-(4-fluoro-7-methyl-5-(4-(((1s,4s)-4-((4-(6-(5-(1-methylcyclopropoxy)-1H-indazol-3-yl)pyrimidin-4-yl)piperazin-1-yl)methyl)cyclohexyl)oxy)piperidin-1-yl)-1-oxoisoindolin-2-yl)piperidine-2,6-dione (16 mg, 18.52 umol, 9.27% yield, 94.93% purity) as white solid.

Exemplary Synthesis of Exemplary Compound 19: Compound 19 was prepared in a manner analogous to compounds 3 and 18.

Exemplary Synthesis of Exemplary Compound 20: Compound 20 was isolated during the preparation of compound 18 (see step 9 for details).

Exemplary Synthesis of Exemplary Compound 21: Compound 21 was prepared in a manner analogous to compound 1 utilizing intermediate 3-(6-chloropyrimidin-4-yl)-5-[(1-methyl cyclopropyl)methyl]-1-tetra hydropyran-2-yl-indazole.

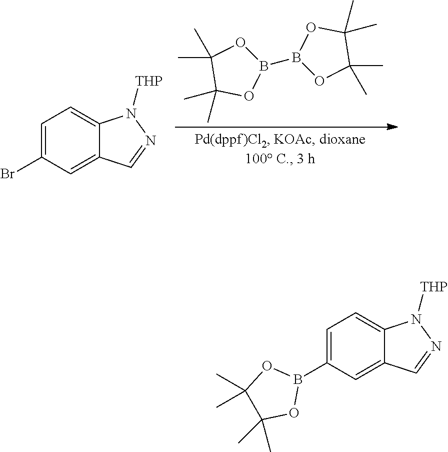

### Step 1:

To a mixture of 4,4,5,5-tetramethyl-2-(4,4,5,5-tetramethyl-1,3,2-dioxaborolan-2-yl)-1,3,2-dioxaborolane (6.77 g, 26.67 mmol, 1.5 eq), [1,1′-bis(diphenylphosphino)ferrocene]dichloropalladium(ii) (1.45 g, 1.78 mmol, 0.1 eq), and 5-bromo-1-tetrahydropyran-2-yl-indazole (5 g, 17.78 mmol, 1 eq) in dioxane (100 mL) was added potassium acetate (3.49 g, 35.56 mmol, 2 eq) in one portion under nitrogen, The mixture was stirred at 100° C. for 3 h and cooled. The reaction mixture was poured into water (10 mL) and the mixture was extracted with ethyl acetate (20 mL×3). The combined organic layers were washed with brine (30 mL×3), dried over anhydrous sodium sulfate, filtered, and concentrated under reduced pressure to give a residue. The residue was purified by silica gel chromatography (Petroleum ether/Ethyl acetate=50/1 to 5/1) to afford 1-tetrahydropyran-2-yl-5-(4,4,5,5-tetramethyl-1,3,2-dioxaborolan-2-yl)indazole (5 g, 15.23 mmol, 86% yield) as yellow gum.

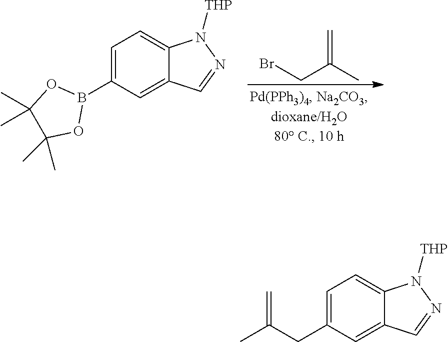

### Step 2:

A mixture of 1-tetrahydropyran-2-yl-5-(4,4,5,5-tetramethyl-1,3,2-dioxaborolan-2-yl)indazole (5 g, 15.23 mmol, 1 eq), 3-bromo-2-methylprop-1-ene (5 g, 37.04 mmol, 3.73 mL, 2.43 eq), tetrakis[triphenylphosphine]palladium(0) (880 mg, 0.76 mmol, 0.05 eq), and sodium carbonate (3.23 g, 30.47 mmol, 2 eq) in dioxane (50 mL) and water (10 mL) was degassed and then heated to 80° C. for 10 h under nitrogen and cooled. The reaction mixture was poured into water (100 mL). The mixture was extracted with ethyl acetate (50 mL×3). The organic phase was washed with brine (50 mL), dried over anhydrous sodium sulfate, filtered, and concentrated under vacuum. The residue was purified by silica gel chromatography (Petroleum ether/Ethyl acetate=50/1 to 5/1) to afford 5-(2-methylallyl)-1-tetrahydropyran-2-yl-indazole (3.2 g, 12.48 mmol, 81% yield) as yellow gum.

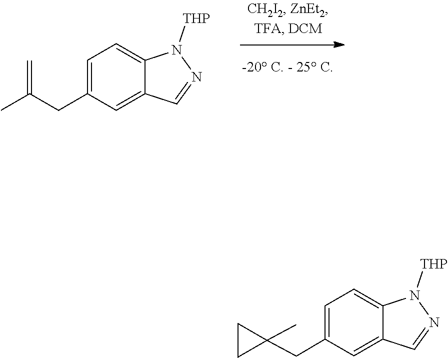

### Step 3:

To a mixture of 5-(2-methylallyl)-1-tetrahydropyran-2-yl-indazole (1 g, 3.90 mmol, 1 eq) and dideuterio(diiodo)methane (2.09 g, 7.80 mmol, 0.62 mL, 2 eq), and trifluoroacetic acid (444 mg, 3.90 mmol, 0.28 mL, 1 eq) in dichloromethane (30 mL) was added diethylzinc (1 M, 7.80 mL, 2 eq) in one portion at −20° C. under nitrogen. The mixture was stirred at 25° C. and stirred for 6 h. The residue was poured into ice-water (w/w=1/1) (50 mL), The aqueous phase was extracted with ethyl acetate (50 mL×2), The combined organic phase was washed with brine (50 mL), dried with anhydrous sodium sulfate, filtered, and concentrated under vacuum. The residue was purified by silica gel chromatography (Petroleum ether/Ethyl acetate=100/1 to 5/1) to afford 5-[(1-methylcyclopropyl)methyl]-1-tetrahydropyran-2-yl-indazole (0.8 g, 2.96 mmol, 75% yield) as yellow oil.

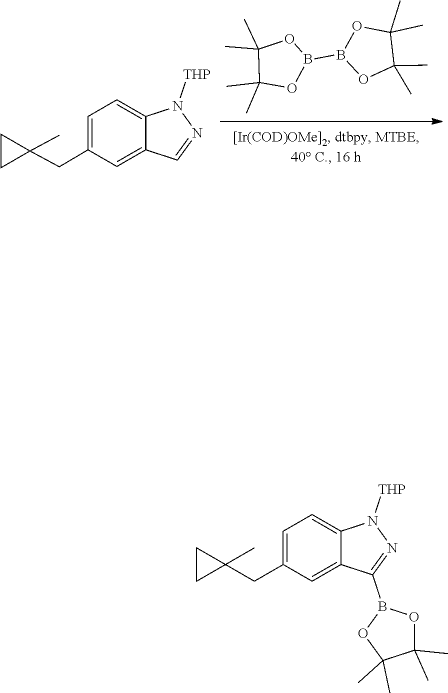

### Step 4:

To a solution of 5-[(1-methylcyclopropyl)methyl]-1-tetrahydropyran-2-yl-indazole (1.7 g, 6.29 mmol, 1 eq) and 4,4,5,5-tetramethyl-2-(4,4,5,5-tetramethyl-1,3,2-dioxaborolan-2-yl)-1,3,2-dioxaborolane (3.19 g, 12.58 mmol, 2 eq) in tetrahydrofuran (17 mL) was added (1,5-cyclooctadiene)(methoxy)iridium(I) dimer (416 mg, 0.62 mmol, 0.1 eq) and 4-tert-butyl-2-(4-tert-butyl-2-pyridyl)pyridine (506 mg, 1.89 mmol, 0.3 eq). The mixture was stirred at 40° C. for 16 h under argon. The reaction mixture was concentrated under reduced pressure to give a residue. The residue was purified by column chromatography (SiO2, Petroleum ether/Ethyl acetate=10/1 to 5/1). Compound 5-[(1-methylcyclopropyl)methyl]-1-tetrahydropyran-2-yl-3-(4,4,5,5-tetramethyl-1,3,2-dioxaborolan-2-yl) indazole (1.9 g, 4.79 mmol, 76% yield) was obtained as a yellow solid.

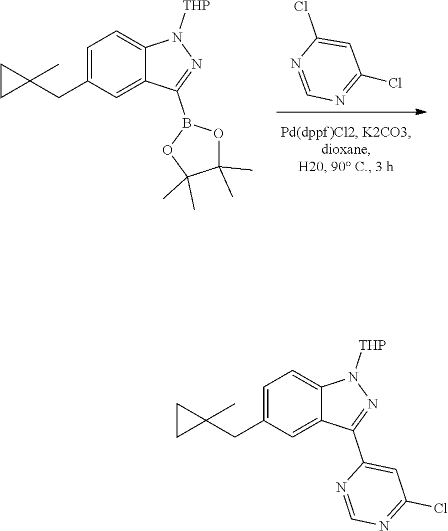

### Step 5

A mixture of 5-[(1-methylcyclopropyl)methyl]-1-tetrahydropyran-2-yl-3-(4,4,5,5-tetramethyl-1,3,2-dioxaborolan-2-yl)indazole (1.6 g, 4.04 mmol, 1 eq), 4,6-dichloropyrimidine (902.15 mg, 6.06 mmol, 1.5 eq), cyclopentyl(diphenyl)phosphane;dichloromethane;dichloropalladium;iron (330 mg, 0.40 mmol, 0.1 eq), potassium carbonate (1.12 g, 8.07 mmol, 2 eq), dioxane (15 mL) and water (5 mL) was degassed with N₂ (3 times), and the mixture was stirred at 90° C. for 3 h under a N₂ atmosphere. The mixture was concentrated under vacuum to remove most of dioxane and the mixture was extracted with ethyl acetate (15 mL*2). The organic phase was washed with brine (10 mL), dried with anhydrous sodium sulfate, filtered, and concentrated under vacuum. The residue was purified by flash silica gel chromatography (ISCO®; 20 g SepaFlash® Silica Flash Column, Eluent of 0-15% Ethylacetate/Petroleum ether gradient @ 45 mL/min). Compound 3-(6-chloropyrimidin-4-yl)-5-[(1-methylcyclopropyl)methyl]-1-tetrahydropyran-2-yl-indazole (1.02 g, 2.66 mmol, 65.99% yield) was obtained as a white solid.

### Exemplary Synthesis of Exemplary Compound 22:

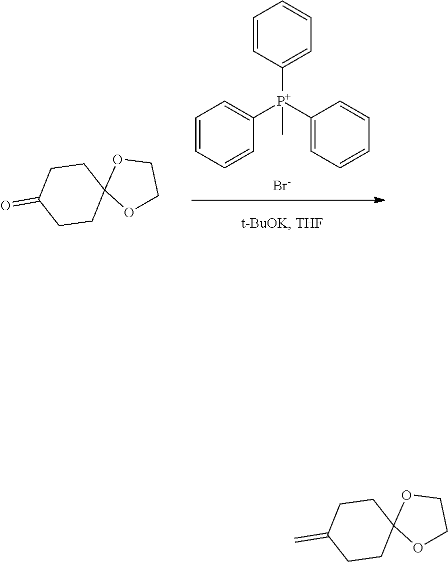 [SMILES: O=C1CCC2(CC1)OCCO2 ; C[P+](c1ccccc1)(c1ccccc1)c1ccccc1 ; [Br-] ; C=C1CCC2(CC1)OCCO2]

### Step 1:

To a solution of methyl(triphenyl)phosphonium;bromide (116.65 g, 326.55 mmol, 1.7 eq) in DMSO (500 mL) was added tBuOK (1 M, 326.55 mL, 1.7 eq) and the solution was stirred at 25° C. for 1 h and cooled to 0° C. 1,4-Dioxaspiro[4.5]decan-8-one (30 g, 192.09 mmol, 1 eq) was added and the mixture was stirred at 25° C. for 15 h. The reaction mixture was quenched by addition ice water (1000 mL), and extracted with EtOAc (500 mL*3). The combined organic layers were washed with brine (500 mL*4), dried over Na₂SO₄, filtered, and concentrated under reduced pressure to give a residue. The residue was purified by flash silica gel chromatography (ISCO®; 220 g SepaFlash® Silica Flash Column, Eluent of 0-7% Ethyl acetate/Petroleum ether gradient @ 100 mL/min). Compound 8-methylene-1,4-dioxaspiro[4.5]decane (28.4 g, 184.17 mmol, 95.88% yield) was obtained as a colorless oil.

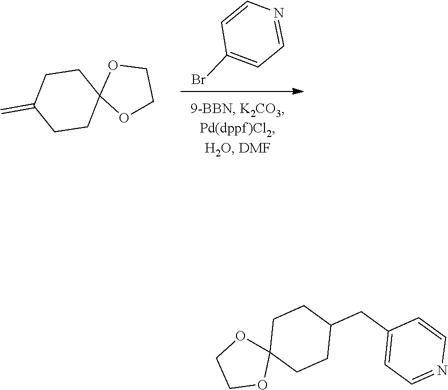 [SMILES: C=C1CCC2(CC1)OCCO2 ; Brc1ccncc1 ; c1cc(CC2CCC3(CC2)OCCO3)ccn1]

### Step 2:

To a solution of 8-methylene-1,4-dioxaspiro[4.5]decane (15 g, 97.27 mmol, 1 eq) was added 9-borabicyclo[3.3.1]nonane (0.5 M, 252.91 mL, 1.3 eq) and the mixture was stirred for 4 h at 80° C. under N₂. The solution was then cooled to 30° C. and 4-bromopyridine (18.44 g, 116.73 mmol, 1.2 eq) was added, followed by K₂CO₃ (40.33 g, 291.82 mmol, 3 eq), DMF (200 mL), H₂O (20 mL) and Pd(dppf)Cl₂ (7.12 g, 9.73 mmol, 0.1 eq). The solution was heated to 80° C. under N₂ for 12 h and cooled. Water (500 mL) was added and the mixture was extracted with EtOAc (300 mL*3). The combined organic layers were washed with brine (3*100 mL), dried over Na₂SO₄, filtered, and concentrated under reduced pressure to give a residue. The residue was purified by flash silica gel chromatography (ISCO®; 120 g SepaFlash® Silica Flash Column, Eluent of 0-62% Ethyl acetate/Petroleum ether gradient @80 mL/min). Compound 4-(1,4-dioxaspiro[4.5]decan-8-ylmethyl)pyridine (17.3 g, 74.15 mmol, 76.23% yield) was obtained as a light yellow oil.

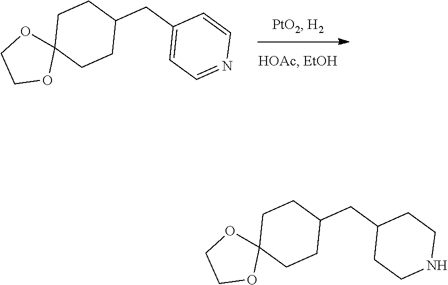 [SMILES: c1cc(CC2CCC3(CC2)OCCO3)ccn1 ; C1CC(CC2CCC3(CC2)OCCO3)CCN1]

### Step 3

To a solution of 4-(1,4-dioxaspiro[4.5]decan-8-ylmethyl)pyridine (1.9 g, 8.14 mmol, 1 eq) in EtOH (50 mL) was added HOAc (978.11 mg, 16.29 mmol, 931.53 uL, 2 eq) and PtO₂ (554.78 mg, 2.44 mmol, 0.3 eq), and the solution was stirred at 60° C. under H₂ (50 Psi) for 16 h and cooled. The reaction mixture was filtered and concentrated under reduced pressure to give 4-(1,4-dioxaspiro[4.5]decan-8-ylmethyl)piperidine (2.3 g, crude) was obtained as a light yellow solid.

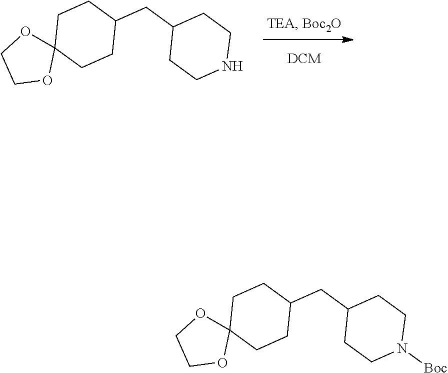 [SMILES: C1CC(CC2CCC3(CC2)OCCO3)CCN1 ; CC(C)(C)OC(=O)N1CCC(CC2CCC3(CC2)OCCO3)CC1]

### Step 4

To a solution of 4-(1,4-dioxaspiro[4.5]decan-8-ylmethyl)piperidine (1.9 g, 7.94 mmol, 1 eq) in DCM (20 mL) was added Boc₂O (3.46 g, 15.88 mmol, 3.65 mL, 2 eq) and TEA (4.02 g, 39.69 mmol, 5.52 mL, 5 eq), and the solution was stirred at 25° C. for 16 h. Water (50 mL) was added and the reaction mixture was extracted with DCM (50 mL*3). The combined organic layers were washed with brine (50 mL), dried over Na₂SO₄, filtered, and concentrated under reduced pressure to give a residue. The residue was purified by flash silica gel chromatography (ISCO®; 40 g SepaFlash® Silica Flash Column, Eluent of 0-19% Ethyl acetate/Petroleum ether gradient @ 80 mL/min). Compound tert-butyl 4-(1,4-dioxaspiro[4.5]decan-8-ylmethyl)piperidine-1-carboxylate (2.3 g, 6.78 mmol, 85.35% yield) was obtained as a brown solid.

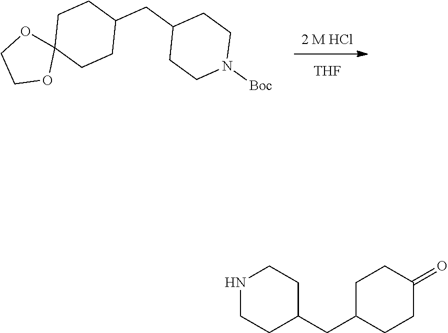 [SMILES: CC(C)(C)OC(=O)N1CCC(CC2CCC3(CC2)OCCO3)CC1 ; O=C1CCC(CC2CCNCC2)CC1 ; "THF"]

### Step 5

To a solution of tert-butyl 4-(1,4-dioxaspiro[4.5]decan-8-ylmethyl)piperidine-1-carboxylate (4.5 g, 13.26 mmol, 1 eq) in THF (60 mL) was added HCl (2 M, 66.28 mL, 10 eq) and the solution was stirred at 60° C. for 8 h and cooled. Na₂CO₃ was added to adjust the pH-10 and the mixture was extracted with DCM (50 mL*3). The combined organic layers were washed with brine (50 mL), dried over Na₂SO₄, filtered, and concentrated under reduced pressure to give 4-(4-piperidylmethyl)cyclohexanone (2.1 g, 10.75 mmol, 81.12% yield) was obtained as a colorless oil.

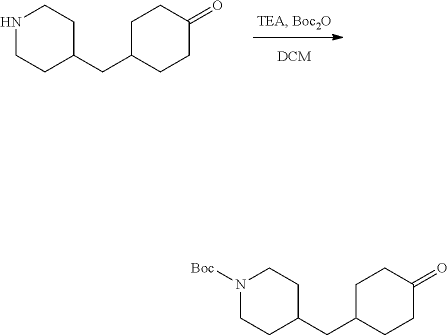 [SMILES: O=C1CCC(CC2CCNCC2)CC1 ; CC(C)(C)OC(=O)N1CCC(CC2CCC(=O)CC2)CC1]

### Step 6

To a solution of 4-(4-piperidylmethyl)cyclohexanone (11 g, 56.32 mmol, 1 eq) in DCM (100 mL) was added Boc₂O (36.88 g, 168.97 mmol, 38.82 mL, 3 eq) and TEA (17.10 g, 168.97 mmol, 23.52 mL, 3 eq) and the solution was stirred at 25° C. for 16 h. Water (300 mL) was added and the mixture was extracted with DCM (300 mL*3). The combined organic layers were washed with brine (200 mL*3), dried over Na₂SO₄, filtered, and concentrated under reduced pressure to give a residue. The residue was purified by flash silica gel chromatography (ISCO®; 12 g SepaFlash® Silica Flash Column, Eluent of 0-40% Ethyl acetate/Petroleum ether gradient @ 70 mL/min) to afford tert-butyl 4-[(4-oxocyclohexyl)methyl]piperidine-1-carboxylate (12 g, 40.62 mmol, 72.12% yield) was obtained as a colorless oil.

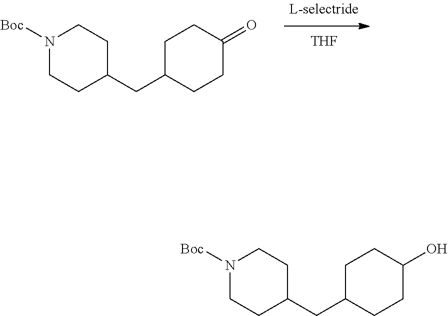 [SMILES: CC(C)(C)OC(=O)N1CCC(CC2CCC(=O)CC2)CC1 ; CC(C)(C)OC(=O)N1CCC(CC2CCC(O)CC2)CC1 ; "THF"]

### Step 7:

To a mixture of tert-butyl 4-[(4-oxocyclohexyl)methyl]piperidine-1-carboxylate (5 g, 16.93 mmol, 1 eq) in THF (250 mL) was added L-selectride (1 M, 25.39 mL, 1.5 eq) at −70° C. over 30 min. The resulting solution was warmed to 25° C. for 16 h under N₂. Water (100 mL) was added and the mixture was extracted with DCM (100 mL*3). The combined organic layers were washed with brine (100 mL*2), dried over Na₂SO₄, filtered, and concentrated under reduced pressure to give a residue. The residue was purified by flash silica gel chromatography (ISCO®; 80 g SepaFlash® Silica Flash Column, Eluent of 0-22% Ethyl acetate/Petroleum ether gradient @ 80 mL/min) to give tert-butyl 4-[(4-hydroxycyclohexyl)methyl]piperidine-1-carboxylate (5 g, 16.81 mmol, 99.32% yield) as colorless gum.

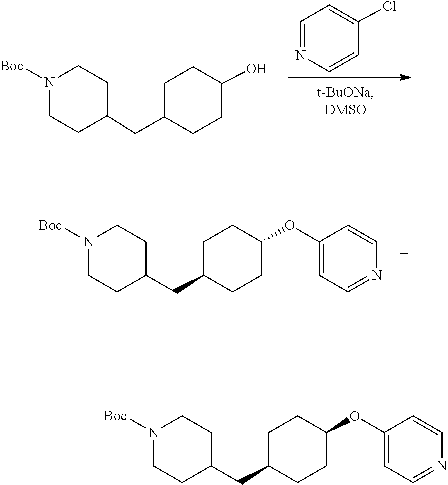 [SMILES: CC(C)(C)OC(=O)N1CCC(CC2CCC(O)CC2)CC1 ; Clc1ccncc1 ; CC(C)(C)OC(=O)N1CCC(C[C@H]2CC[C@H](Oc3ccncc3)CC2)CC1 ; CC(C)(C)OC(=O)N1CCC(C[C@H]2CC[C@@H](Oc3ccncc3)CC2)CC1]

### Step 8

To a solution of tBuONa (2.58 g, 26.90 mmol, 4 eq) in DMSO (40 mL) was added tert-butyl 4-[(4-hydroxycyclohexyl)methyl]piperidine-1-carboxylate (2.00 g, 6.72 mmol, 1 eq) and 4-chloropyridine (1.51 g, 10.09 mmol, 1.5 eq, HCl) at 40° C., and the reaction mixture was stirred at 80° C. for 16 h. The mixture was poured into ice-water (100 mL). The aqueous phase was extracted with ethyl acetate (80 mL*3). The combined organic phase was washed with brine (50 mL*3), dried with anhydrous Na₂SO₄, filtered, and concentrated under vacuum. The residue was purified by flash silica gel chromatography (ISCO®; 80 g SepaFlash® Silica Flash Column, Eluent of 0-100% Ethylacetate/Petroleum ether gradient @ 60 mL/min) to afford tert-butyl 4-[[4-(4-pyridyloxy)cyclohexyl]methyl]piperidine-1-carboxylate (1.9 g, 3.96 mmol, 58.85% yield, 78% purity) as white solid. The resulting residue was purified by prep-SFC (column: Phenomenex-Cellulose-2 (250 mm*30 mm, 10 um); mobile phase: [0.1% NH3H2O MEOH]; B %: 40%-40%, min) to separate the cis and trans isomers: Peak 1: RT=1.657 min tert-butyl 4-[[4-(4-pyridyloxy)cyclohexyl]methyl]piperidine-1-carboxylate (630 mg, 1.68 mmol, 63.00% yield); Peak 2: RT=1.561 min tert-butyl 4-[[4-(4-pyridyloxy)cyclohexyl]methyl]piperidine-1-carboxylate (270 mg, 720.93 umol, 27.00% yield) as yellow solid. Peak 1 was designated the cis isomer.

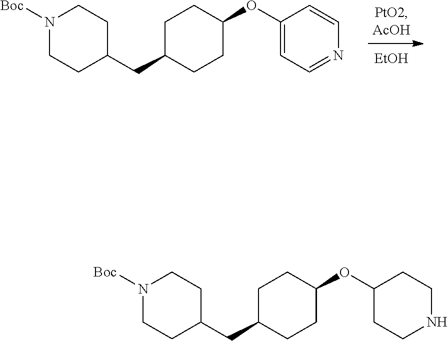 [SMILES: "EtOH" ; CC(C)(C)OC(=O)N1CCC(C[C@H]2CC[C@@H](Oc3ccncc3)CC2)CC1 ; CC(C)(C)OC(=O)N1CCC(C[C@H]2CC[C@@H](OC3CCNCC3)CC2)CC1]

### Step 9

To a mixture of tert-butyl 4-[[4-(4-pyridyloxy)cyclohexyl]methyl]piperidine-1-carboxylate (600 mg, 1.60 mmol, 1 eq) in EtOH (20 mL) was added PtO₂ (72.76 mg, 320.41 umol, 0.2 eq) and AcOH (288.61 mg, 4.81 mmol, 274.87 uL, 3 eq) and the mixture was heated to 70° C. under H₂ (50 PSI) for 48 h and cooled. The residue was filtered and concentrated under vacuum. The resulting product was poured into NaHCO₃ (pH=7) and the mixture was extracted with DCM (20 mL*3). The organic phase was washed with brine (50 mL), dried over anhydrous Na₂SO₄, and concentrated under vacuum to give tert-butyl 4-[[4-(4-piperidyloxy)cyclohexyl]methyl]piperidine-1-carboxylate (560 mg, 1.47 mmol, 91.85% yield) as a colorless oil.

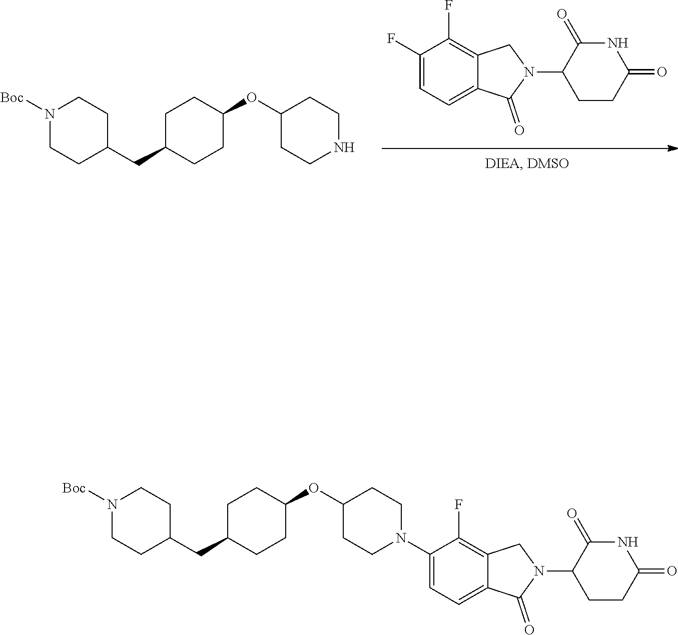 [SMILES: CC(C)(C)OC(=O)N1CCC(C[C@H]2CC[C@@H](OC3CCNCC3)CC2)CC1 ; O=C1CCC(N2Cc3c(ccc(F)c3F)C2=O)C(=O)N1 ; CC(C)(C)OC(=O)N1CCC(C[C@H]2CC[C@@H](OC3CCN(c4ccc5c(c4F)CN(C4CCC(=O)NC4=O)C5=O)CC3)CC2)CC1]

### Step 10

A mixture of tert-butyl 4-[[4-(4-piperidyloxy)cyclohexyl]methyl]piperidine-1-carboxylate (128.51 mg, 337.69 umol, 1 eq) and 3-(4,5-difluoro-1-oxo-isoindolin-2-yl)piperidine-2,6-dione (75.70 mg, 270.15 umol, 0.8 eq) in DMSO (10 mL) was added DIEA (218.22 mg, 1.69 mmol, 294.10 uL, 5 eq) and the mixture was stirred at 130° C. for 12 h under N₂ atmosphere and cooled. The resulting mixture was poured into H₂O at 0° C. The mixture was extracted with Ethyl acetate (20 mL*3). The organic phase was washed with brine (20 mL), dried over anhydrous Na₂SO₄, and concentrated under vacuum to give a residue. The residue was purified by silica gel column chromatography (0 to 100% Ethyl acetate in Petroleum ether) to give tert-butyl 4-[[4-[[1-[2-(2,6-dioxo-3-piperidyl)-4-fluoro-1-oxo-isoindolin-5-yl]-4-piperidyl]oxy]cyclohexyl]methyl]piperidine-1-carboxylate (100 mg, 87.39 umol, 25.88% yield, 56% purity) as a white solid

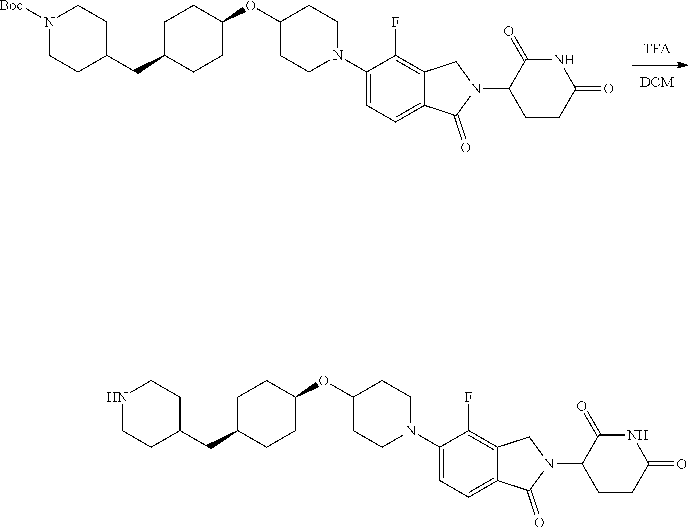 [SMILES: CC(C)(C)OC(=O)N1CCC(C[C@H]2CC[C@@H](OC3CCN(c4ccc5c(c4F)CN(C4CCC(=O)NC4=O)C5=O)CC3)CC2)CC1 ; O=C1CCC(N2Cc3c(ccc(N4CCC(O[C@H]5CC[C@@H](CC6CCNCC6)CC5)CC4)c3F)C2=O)C(=O)N1]

### Step 11

A solution of tert-butyl 4-[[4-[[1-[2-(2,6-dioxo-3-piperidyl)-4-fluoro-1-oxo-isoindolin-5-yl]-4-piperidyl]oxy]cyclohexyl]methyl]piperidine-1-carboxylate (100 mg, 156.06 umol, 1 eq) in DCM (5 mL) was added TFA (3.08 g, 27.01 mmol, 2 mL, 173.09 eq) and then the mixture was stirred at 25° C. for 1 h under a N₂ atmosphere. The reaction mixture was concentrated under reduced pressure to give 3-[4-fluoro-1-oxo-5-[4-[4-(4-piperidylmethyl)cyclohexoxy]-1-piperidyl]isoindolin-2-yl]piperidine-2,6-dione (100 mg, crude, TFA) as a white solid

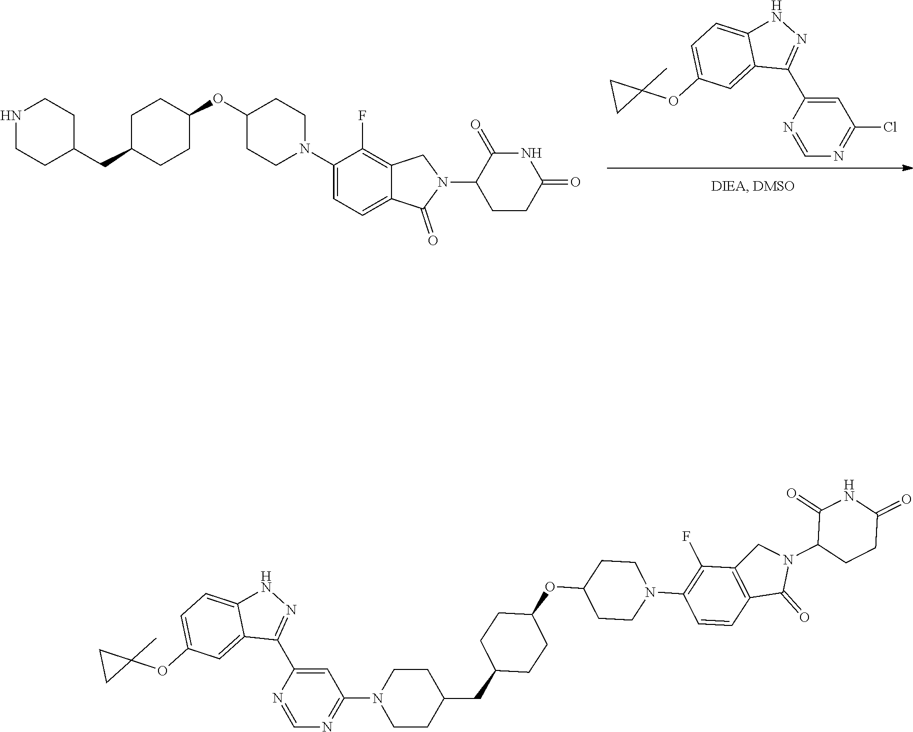 [SMILES: O=C1CCC(N2Cc3c(ccc(N4CCC(O[C@H]5CC[C@@H](CC6CCNCC6)CC5)CC4)c3F)C2=O)C(=O)N1 ; CC1(Oc2ccc3[nH]nc(-c4cc(Cl)ncn4)c3c2)CC1 ; CC1(Oc2ccc3[nH]nc(-c4cc(N5CCC(C[C@H]6CC[C@@H](OC7CCN(c8ccc9c(c8F)CN(C8CCC(=O)NC8=O)C9=O)CC7)CC6)CC5)ncn4)c3c2)CC1]

### Step 12:

A mixture of 3-[4-fluoro-1-oxo-5-[4-[4-(4-piperidylmethyl)cyclohexoxy]-1-piperidyl]isoindolin-2-yl]piperidine-2,6-dione (89.89 mg, 166.26 umol, 1 eq) and 3-(6-chloropyrimidin-4-yl)-5-(1-methylcyclopropoxy)-1H-indazole (50 mg, 166.26 umol, 1 eq) in DMSO (5 mL) was added DIEA (64.46 mg, 498.77 umol, 86.87 uL, 3 eq) and the mixture was stirred at 80° C. for 12 hr under N₂ atmosphere and cooled. The residue was purified by prep-HPLC (column: Phenomenex C18 75*30 mm*3 um; mobile phase: [water(FA)-ACN]; B %: 26%-66%, 26 min) to give 3-[4-fluoro-5-[4-[4-[[1-[6-[5-(1-methylcyclopropoxy)-1H-indazol-3-yl]pyrimidin-4-yl]-4-piperidyl]methyl]cyclohexoxy]-1-piperidyl]-1-oxo-isoindolin-2-yl]piperidine-2,6-dione (21.2 mg, 25.28 umol, 15.21% yield, 96% purity) as a white solid.

Exemplary Synthesis of Exemplary Compound 23: Compound 23 was prepared in a manner analogous to compounds 22 utilizing peak 2 (step 8) intermediate tert-butyl 4-[[4-(4-pyridyloxy)cyclohexyl]methyl]piperidine-1-carboxylate.

Exemplary Synthesis of Exemplary Compound 24: Compound 24 was prepared in a manner analogous to compound 21.

Exemplary Synthesis of Exemplary Compound 25: Compound 25 was prepared in a manner analogous to compound 21.

Exemplary Synthesis of Exemplary Compound 26: Compound 26 was prepared in a manner analogous to compound 21.

Exemplary Synthesis of Exemplary Compound 27: Compound 27 was prepared in a manner analogous to compound 22.

Exemplary Synthesis of Exemplary Compound 28: Compound 28 was prepared in a manner analogous to compounds 4 and 21.

Exemplary Synthesis of Exemplary Compound 29: Compound 29 was prepared in a manner analogous to compounds 22 and 23.

Exemplary Synthesis of Exemplary Compound 30: Compound 30 was prepared in a manner analogous to compound 21.

Characterization data for the compounds disclosed herein is provided in Table 1.

TABLE 1

| | 1H NMR | | | |
|---|---|---|---|---|
| | 1H NMR: (400 MHz, DMSO-d6) or | Mol | Exact | |
| Ex. | 1H NMR: (400 MHz, CD3OD) | Weight | Mass | [M + H]⁺ |
| 1 | 1H NMR (400 MHz, DMSO-d6): δ 13.43 (s, 1H), 10.98 | 806.93 | 806.40 | 807.3 |
| | (s, 1H), 8.68 (s, 1H), 8.15 (d, J = 1.6 Hz, 1H), 8.13 (s, | | | |
| | 1H), 7.51-7.47 (m, 2H), 7.41 (s, 1H), 7.16 (t, J = 8.0 | | | |
| | Hz, 1H), 7.07 (dd, J = 2.4, 8.8 Hz, 1H), 5.06 (dd, J = | | | |
| | 5.2, 12.8 Hz, 1H), 4.47 (m, 1H), 4.30 (m, 3H), 4.01-3.93 | | | |
| | (m, 1H), 3.79-3.68 (m, 1H), 3.62-3.55 (m, 1H), | | | |
| | 3.49 (m, 3H), 3.08-2.98 (m, 2H), 2.96-2.83 (m, 3H), | | | |
| | 2.83-2.72 (m, 6H), 2.62-2.53 (m, 6H), 2.44-2.34 | | | |
| | (m, 1H), 1.97 (m, 1H), 1.87-1.71 (m, 3H), 1.54 (s, | | | |
| | 3H), 1.30 (m, 2H), 0.98-0.90 (m, 2H), 0.80-0.73 (m, 2H) | | | |
| 2 | 1H NMR (400 MHz, DMSO-d6): δ 13.76-13.46 (m, | 806.93 | 806.40 | 807.6 |
| | 1H), 11.11-10.94 (m, 1H), 8.76-8.67 (m, 1H), 8.15-8.13 | | | |
| | (m, 1H), 8.10 (br s, 1H), 7.55 (d, J = 9.2 Hz, 1H), | | | |
| | 7.49 (d, J = 8.0 Hz, 1H), 7.44 (s, 1H), 7.18 (t, J = 8.4 | | | |
| | Hz, 1H), 7.12 (dd, J = 2.0, 8.4 Hz, 1H), 5.13-5.03 (m, | | | |
| | 1H), 4.54-4.42 (m, 2H), 4.37-4.27 (m, 2H), 4.09-3.96 | | | |
| | (m, 3H), 3.94-3.73 (m, 4H), 3.70-3.59 (m, 2H), | | | |
| | 3.57-3.46 (m, 3H), 3.18-3.03 (m, 3H), 2.96-2.75 | | | |
| | (m, 7H), 2.64-2.55 (m, 2H), 2.46-2.38 (m, 1H), 2.03-1.93 | | | |
| | (m, 1H), 1.88-1.81 (m, 2H), 1.55 (s, 3H), 1.45-1.28 | | | |
| | (m, 2H), 0.99-0.91 (m, 2H), 0.82-0.72 (m, 2H). | | | |
| 3 | 1H NMR (400 MHz, DMSO-d6): δ 13.44 (s, 1H), 10.99 | 834.98 | 834.43 | 835.3 |
| | (s, 1H), 8.69 (s, 1H), 8.18-8.14 (m, 1H), 8.14 (s, 1H), | | | |
| | 7.55-7.45 (m, 2H), 7.40 (s, 1H), 7.16 (t, J = 8.0 Hz, | | | |
| | 1H), 7.08 (dd, J = 2.4, 9.2 Hz, 1H), 5.07 (dd, J = 5.2, | | | |
| | 13.2 Hz, 1H), 4.48 (m, 1H), 4.30 (m, 3H), 3.98 m, 1H), | | | |
| | 3.67-3.44 (m, 4H), 3.08-2.97 (m, 1H), 2.96-2.85 | | | |
| | (m, 2H), 2.83-2.70 (m, 4H), 2.63-2.55 (m, 2H), 2.54-2.51 | | | |
| | (m, 2H), 2.44-2.36 (m, 2H), 2.25-2.11 (m, 3H), | | | |
| | 2.01-1.93 (m, 1H), 1.82 (m, 2H), 1.76-1.65 (m, 1H), | | | |
| | 1.54 (s, 3H), 1.34-1.20 (m, 3H), 1.20-0.99 (m, 6H), | | | |
| | 0.97-0.92 (m, 2H), 0.81-0.73 (m, 2H) | | | |
| 4 | 1H NMR (400 MHz, DMSO-d6): δ 13.42-13.34 (m, | 815.96 | 815.41 | 816.7 |
| | 1H), 10.98-10.92 (m, 1H), 8.59 (s, 1H), 8.20-8.18 | | | |
| | (m, 1H), 8.15 (s, 1H), 7.54-7.46 (m, 1H), 7.41-7.34 | | | |
| | (m, 1H), 7.13-7.03 (m, 2H), 7.00-6.92 (m, 1H), 5.10-5.03 | | | |
| | (m, 1H), 4.44 (d, J = 16.8 Hz, 1H), 4.26 (d, J = | | | |
| | 16.9 Hz, 1H), 4.19-4.13 (m, 1H), 3.87-3.84 (m, 3H), | | | |
| | 3.79 (s, 4H), 2.96-2.74 (m, 6H), 2.63-2.56 (m, 1H), | | | |
| | 2.46-2.43 (m, 3H), 2.21-2.09 (m, 4H), 2.02-1.92 | | | |
| | (m, 6H), 1.77 (br s, 4H), 1.64-1.57 (m, 2H), 1.54 (s, | | | |
| | 3H), 0.96-0.93 (m, 2H), 0.79-0.75 (m, 2H) | | | |
| 5 | 1H NMR (400 MHz, DMSO-d6): δ 13.40 (br s, 1H), | 820.95 | 820.42 | 821.3 |
| | 10.98 (s, 1H), 8.64 (s, 1H), 8.15 (d, J = 2.0 Hz, 1H), | | | |
| | 8.14 (s, 1H), 7.55-7.46 (m, 2H), 7.38 (s, 1H), 7.15 (t, | | | |
| | J = 8.0 Hz, 1H), 7.07 (dd, J = 2.4, 8.8 Hz, 1H), 5.07 | | | |
| | (dd, J = 5.2, 13.2 Hz, 1H), 4.47 (m, 1H), 4.30 (m, 1H), | | | |
| | 3.78 m, 1H), 3.71-3.60 (m, 5H), 3.59-3.43 (m, 4H), | | | |
| | 2.96-2.82 (m, 2H), 2.80-2.69 (m, 3H), 2.60 (m 1H), | | | |
| | 2.59-2.54 (m, 3H), 2.45-2.34 (m, 5H), 2.05-1.92 | | | |
| | (m, 2H), 1.84-1.72 (m, 3H), 1.54 (s, 3H), 1.45 (br s, | | | |
| | 3H), 1.39-1.26 (m, 2H), 0.98-0.90 (m, 2H), 0.80-0.73 | | | |
| | (m, 2H) | | | |
| 6 | 1H NMR (400 MHz, DMSO-d6): δ 13.11-13.67 (1 H, | 836.97 | 836.43 | 837.3 |
| | m), 10.97 (1 H, s), 8.65 (1 H, d, J = 0.88 Hz), | | | |
| | 8.09-8.22 (2 H, m), 7.48-7.53 (1 H, m), 7.42-7.48 (2 H, | | | |
| | m), 7.11-7.17 (1 H, m), 7.04-7.09 (1 H, m), 5.02-5.11 | | | |
| | (1 H, m), 4.47 (1 H, d, J = 16.88 Hz), 4.30 (3 H, | | | |
| | d, J = 16.88 Hz), 3.45 (3 H, d, J = 3.76 Hz), 3.25 (3 H, | | | |
| | s), 2.85-2.96 (1 H, m), 2.74 (2 H, s), 2.52-2.62 (4 H, | | | |
| | m), 2.35-2.44 (6 H, m), 2.08-2.14 (2 H, m), 1.93-2.00 | | | |
| | (1 H, m), 1.63-1.89 (9 H, m), 1.54 (3 H, s), 1.15-1.28 | | | |
| | (2 H, m), 0.92-0.97 (2 H, m), 0.75-0.80 (2 H, m). | | | |
| 7 | 1H NMR (400 MHz, DMSO-d6): δ 13.37 (s, 1H), 10.98 | 822.94 | 822.41 | 823.3 |
| | (s, 1H), 8.62 (s, 1H), 8.18-8.12 (m, 1H), 7.53-7.45 | | | |
| | (m, 2H), 7.37 (s, 1H), 7.16 (t, J = 8.1 Hz, 1H), | | | |
| | 7.09-7.02 (m, 1H), 5.13-5.02 (m, 1H), 4.84-4.64 (m, 1H), | | | |
| | 4.54-4.26 (m, 4H), 3.15 (br s, 4H), 3.01-2.85 (m, | | | |
| | 4H), 2.63-2.53 (m, 4H), 2.45-2.36 (m, 4H), 2.28-2.09 | | | |
| | (m, 4H), 2.02-1.92 (m, 2H), 1.89-1.73 (m, 4H), | | | |
| | 1.58-1.45 (m, 5H), 1.14-1.01 (m, 2H), 0.97-0.90 | | | |
| | (m, 2H), 0.80-0.71 (m, 2H) | | | |
| 8 | 1H NMR (400 MHz, DMSO-d6): δ 13.43 (1 H, s), 10.99 | 819.97 | 819.43 | 820.5 |
| | (1 H, s), 8.66 (1 H, s), 8.13-8.16 (1 H, m), 7.45-7.55 | | | |
| | (2 H, m), 7.38-7.44 (1 H, m), 7.12-7.20 (1 H, m), | | | |
| | 7.08 (1 H, dd, J = 9.05, 2.32 Hz), 5.07 (1 H, dd, J = | | | |
| | 13.57, 5.01 Hz), 4.48 (1 H, d, J = 16.99 Hz), 4.30 (1 H, | | | |
| | d, J = 16.87 Hz), 3.69-3.77 (4 H, m), 3.45-3.53 | | | |
| | (8 H, m), 3.06-3.24 (5 H, m), 2.86-2.97 (2 H, m), | | | |
| | 2.73-2.82 (3 H, m), 2.57-2.64 (5 H, m), 2.43 (3 H, d, | | | |
| | J = 4.52 Hz), 1.94-1.99 (1 H, m), 1.78-1.86 (2 H, m), | | | |
| | 1.54 (3 H, s), 1.23-1.35 (2 H, m), 0.91-0.97 (2 H, | | | |
| | m), 0.75-0.80 (2 H, m) | | | |
| 9 | 1H NMR (400 MHz, DMSO-d6): δ 13.40 (br s, 1H), | 822.94 | 822.41 | 823.2 |
| | 10.99 (s, 1H), 8.65 (s, 1H), 8.15 (s, 2H), 7.54-7.43 | | | |
| | (m, 2H), 7.39 (s, 1H), 7.17 (t, J = 7.8 Hz, 1H), | | | |
| | 7.10-7.03 (m, 1H), 5.12-5.03 (m, 1H), 4.83-4.67 (m, 1H), | | | |
| | 4.48 (br d, J = 16.9 Hz, 1H), 4.31 (br d, J = 16.8 Hz, | | | |
| | 1H), 3.68 (br s, 4H), 3.17-3.02 (m, 4H), 2.97-2.85 | | | |
| | (m, 3H), 2.78 (br t, J = 11.5 Hz, 2H), 2.61 (brd, J = 2.3 | | | |
| | Hz, 2H), 2.41 (br s, 2H), 2.26-2.10 (m, 4H), 2.09-1.91 | | | |
| | (m, 3H), 1.86-1.67 (m, 4H), 1.59-1.46 (m, 5H), | | | |
| | 1.31-1.20 (m, 2H), 0.98-0.90 (m, 2H), 0.80-0.73 (m, 2H) | | | |
| 10 | 1H NMR (400 MHz, DMSO-d6): δ 13.40 (s, 1H), 10.98 | 806.93 | 806.40 | 807.3 |
| | (s, 1H), 8.65 (s, 1H), 8.26-7.94 (m, 1H), 7.64-7.45 | | | |
| | (m, 2H), 7.39 (s, 1H), 7.18 (br s, 1H), 7.07 (dd, J = 2.4, | | | |
| | 9.2 Hz, 1H), 5.08 (dd, J = 4.8, 13.4 Hz, 1H), 4.64-4.22 | | | |
| | (m, 2H), 4.06-3.93 (m, 1H), 3.67 (br d, J = 1.2 Hz, | | | |
| | 4H), 3.53-3.41 (m, 3H), 3.24-2.59 (m, 12H), 2.40-2.28 | | | |
| | (m, 2H), 2.23-2.06 (m, 2H), 2.01-1.61 (m, 4H), | | | |
| | 1.54 (s, 3H), 1.34-1.12 (m, 2H), 1.00-0.91 (m, 2H), | | | |
| | 0.87-0.70 (m, 2H) | | | |
| 11 | 1H NMR (400 MHz, DMSO-d6): δ 13.38 (s, 1H), 10.98 | 833.99 | 833.44 | 834.4 |
| | (s, 1H), 8.64 (s, 1H), 8.16 (d, J = 2.0 Hz, 1H), 8.14 (s, | | | |
| | 1H), 7.51 (d, J = 8.8 Hz, 1H), 7.52-7.46 (m, 1H), 7.40 | | | |
| | (s, 1H), 7.19-7.14 (m, 1H), 7.09-7.05 (m, 1H), 5.08 | | | |
| | (dd, J = 4.8, 13.2 Hz, 1H), 4.48 (d, J = 16.8 Hz, 1H), | | | |
| | 4.31 (d, J = 16.8 Hz, 1H), 4.15-4.00 (m, 2H), 3.79-3.69 | | | |
| | (m, 1H), 3.69-3.58 (m, 1H), 3.52-3.46 (m, 2H), | | | |
| | 3.40-3.35 (m, 2H), 2.96-2.85 (m, 1H), 2.83-2.71 | | | |
| | (m, 2H), 2.64-2.58 (m, 2H), 2.44-2.40 (m, 3H), 1.99-1.85 | | | |
| | (m, 6H), 1.77-1.67 (m, 2H), 1.58-1.54 (m, 4H), | | | |
| | 1.49-1.39 (m, 3H), 1.36-1.24 (m, 3H), 1.15-1.03 | | | |
| | (m, 3H), 1.03-0.88 (m, 5H), 0.80-0.75 (m, 2H) | | | |
| 12 | 1H NMR (400 MHz, CD3OD): δ 8.64 (s, 1H), 8.41 (s, | 806.93 | 806.40 | 807.3 |
| | 1H), 8.11 (s, 1H), 7.58-7.39 (m, 3H), 7.23-7.06 (m, | | | |
| | 2H), 5.11 (dd, J = 4.8, 13.2 Hz, 1H), 4.58-4.40 (m, | | | |
| | 2H), 3.96-3.71 (m, 7H), 3.59 (br d, J = 9.0 Hz, 2H), | | | |
| | 2.96-2.72 (m, 10H), 2.57-2.46 (m, 2H), 2.37 (br d, J = | | | |
| | 6.8 Hz, 2H), 2.27-2.10 (m, 2H), 2.05-1.86 (m, 5H), | | | |
| | 1.61 (s, 3H), 1.41 (br d, J = 10.8 Hz, 2H), 1.05-0.97 | | | |
| | (m, 2H), 0.82-0.75 (m, 2H) | | | |
| 13 | 1H NMR (400 MHz, DMSO-d6): δ 13.41-13.35 (m, | 806.93 | 806.40 | 807.4 |
| | 1H), 10.96 (s, 1H), 8.64 (s, 1H), 8.17-8.13 (m, 1H), | | | |
| | 7.54-7.43 (m, 2H), 7.38 (s, 1H), 7.16 (t, J = 8.0 Hz, | | | |
| | 1H), 7.07 (dd, J = 2.4, 9.2 Hz, 1H), 5.06 (dd, J = 5.2, | | | |
| | 13.4 Hz, 1H), 4.47 (m, 1H), 4.31 (m, 1H), 3.77 (m, 2H), | | | |
| | 3.66 (m, 5H), 3.55-3.45 (m, 3H), 2.97-2.85 (m, 2H), | | | |
| | 2.83-2.73 (m, 4H), 2.67 (m, 2H), 2.63-2.60 (m, 1H), | | | |
| | 2.43-2.38 (m, 2H), 2.37-2.31 (m, 1H), 2.19 (m, 2H), | | | |
| | 2.03-1.94 (m, 2H), 1.88-1.77 (m, 3H), 1.76 (m, 1H), | | | |
| | 1.54 (s, 3H), 1.28 (m, 2H), 0.98-0.90 (m, 2H), 0.81-0.73 | | | |
| | (m, 2H) | | | |
| 14 | 1H NMR (400 MHz, DMSO-d6): δ 13.39 (br s, 1H), | 787.95 | 787.42 | 788.3 |
| | 10.94 (s, 1H), 8.64 (s, 1H), 8.17-8.14 (m, 1H), 7.50 | | | |
| | (dd, J = 3.4, 8.6 Hz, 2H), 7.37 (s, 1H), 7.08-7.04 (m, | | | |
| | 3H), 5.06-5.01 (m, 1H), 4.33-4.28 (m, 1H), 4.21-4.16 | | | |
| | (m, 1H), 3.66 (br s, 7H), 3.07-3.02 (m, 2H), 2.94-2.86 | | | |
| | (m, 1H), 2.56 (br s, 2H), 2.41-2.33 (m, 5H), 2.11 | | | |
| | (br d, J = 5.8 Hz, 2H), 1.96-1.91 (m, 3H), 1.86-1.78 | | | |
| | (m, 4H), 1.54 (s, 3H), 1.47 (br d, J = 9.1 Hz, 3H), | | | |
| | 1.16 (br d, J = 11.5 Hz, 2H), 0.96-0.90 (m, 4H), 0.78-0.75 | | | |
| | (m, 2H) | | | |
| 15 | 1H NMR (400 MHz, DMSO-d6): δ 13.33-13.45 (1 H, | 854.96 | 854.42 | 855.4 |
| | m), 10.87-11.05 (1 H, m), 8.65 (1 H, s), 8.08-8.24 | | | |
| | (1 H, m), 7.43-7.54 (2 H, m), 7.35-7.41 (1 H, m), | | | |
| | 7.13-7.21 (1 H, m), 7.04-7.10 (1 H, m), 5.07 (1 H, | | | |
| | dd, J = 13.27, 5.20 Hz), 4.41-4.53 (1 H, m), 4.24-4.37 | | | |
| | (1 H, m), 3.63-3.76 (4 H, m), 3.45-3.56 (2 H, | | | |
| | m), 3.30 (4 H, s), 2.94 (6 H, s), 2.59-2.69 (5 H, m), | | | |
| | 2.52-2.58 (1 H, m), 2.35-2.43 (2 H, m), 2.08-2.22 | | | |
| | (1 H, m), 1.74-1.98 (6 H, m), 1.57-1.67 (1 H, m), | | | |
| | 1.54 (3 H, s), 1.25-1.39 (2 H, m), 0.87-0.98 (2 H, | | | |
| | m), 0.72-0.81 (2 H, m) | | | |
| 16 | 1H NMR (400 MHz, DMSO-d6): δ 13.43 (s, 1H), 10.98 | 834.98 | 834.43 | 835.3 |
| | (s, 1H), 8.69 (s, 1H), 8.20-8.14 (m, 1H), 7.55-7.45 | | | |
| | (m, 2H), 7.40 (s, 1H), 7.16 (t, J = 8.0 Hz, 1H), 7.08 (dd, | | | |
| | J = 2.4, 9.2 Hz, 1H), 5.07 (dd, J = 5.2, 13.2 Hz, 1H), | | | |
| | 4.48 (m, 1H), 4.43-4.15 (m, 3H), 3.97 (m, 1H), 3.64-3.43 | | | |
| | (m, 4H), 3.08-2.97 (m, 1H), 2.98-2.84 (m, 2H), | | | |
| | 2.83-2.70 (m, 4H), 2.63-2.59 (m, 1H), 2.58-2.55 | | | |
| | (m, 1H), 2.54-2.51 (m, 2H), 2.44-2.36 (m, 2H), 2.25-2.08 | | | |
| | (m, 3H), 2.01-1.93 (m, 1H), 1.82 (m, 2H), 1.76-1.65 | | | |
| | (m, 1H), 1.54 (s, 3H), 1.38-0.97 (m, 9H), 0.97-0.92 | | | |
| | (m, 2H), 0.81-0.73 (m, 2H) | | | |
| 17 | 1H NMR (400 MHz, DMSO-d6): δ 13.39 (br s, 1H), | 820.95 | 820.42 | 821.3 |
| | 10.97 (s, 1H), 8.64 (d, J = 0.8 Hz, 1H), 8.15 (d, J = 2.0 | | | |
| | Hz, 1H), 8.14 (s, 1H), 7.51-7.40 (m, 2H), 7.37 (s, 1H), | | | |
| | 7.14 (t, J = 7.8 Hz, 1H), 7.06 (dd, J = 2.4, 9.2 Hz, 1H), | | | |
| | 5.06 (dd, J = 5.2, 13.2 Hz, 1H), 4.47 (m, 1H), 4.30 (m, | | | |
| | 1H), 3.78 (br d, J = 11.1 Hz, 1H), 3.75-3.6 (m, 5H), | | | |
| | 3.56-3.41 (m, 4H), 2.96-2.81 (m, 2H), 2.80-2.69 | | | |
| | (m, 3H), 2.60 (m, 1H), 2.58-2.52 (m, 3H), 2.45-2.33 | | | |
| | (m, 5H), 2.05-1.92 (m, 2H), 1.85-1.73 (m, 3H), 1.54 | | | |
| | (s, 3H), 1.44 (m, 3H), 1.39-1.25 (m, 2H), 0.97-0.91 | | | |
| | (m, 2H), 0.79-0.73 (m, 2H) | | | |
| 18 | 1H NMR (400 MHz, DMSO-d6): δ 13.41 (br s, 1H), | 819.96 | 819.42 | 820.3 |
| | 10.98 (s, 1H), 8.65 (s, 1H), 8.26-8.21 (m, 1H), 8.16 | | | |
| | (d, J = 1.6 Hz, 1H), 7.51 (d, J = 9.0 Hz, 1H), 7.38 (s, | | | |
| | 1H), 7.10-7.03 (m, 1H), 6.91 (d, J = 7.5 Hz, 1H), | | | |
| | 5.09-4.97 (m, 1H), 4.40 (d, J = 17.0 Hz, 1H), 4.24 (d, J = | | | |
| | 16.9 Hz, 1H), 3.73-3.57 (m, 5H), 3.43-3.37 (m, 2H), | | | |
| | 3.02-2.84 (m, 3H), 2.63-2.56 (m, 1H), 2.54 (s, 4H), | | | |
| | 2.46-2.35 (m, 5H), 2.13 (br d, J = 6.9 Hz, 2H), 2.01-1.87 | | | |
| | (m, 4H), 1.82 (br d, J = 11.0 Hz, 2H), 1.62-1.45 | | | |
| | (m, 6H), 1.24-1.10 (m, 2H), 1.00-0.88 (m, 4H), 0.80-0.73 | | | |
| | (m, 2H) | | | |
| 19 | 1H NMR (400 MHz, DMSO-d6): δ 13.52-13.26 (m, | 847.03 | 846.47 | 847.9 |
| | 1H), 11.06-10.82 (m, 1H), 8.73-8.58 (m, 1H), 8.24-8.04 | | | |
| | (m, 1H), 7.55-7.48 (m, 1H), 7.42-7.35 (m, 1H), | | | |
| | 7.09-7.04 (m, 1H), 6.92-6.84 (m, 1H), 5.11-4.96 | | | |
| | (m, 1H), 4.54-4.43 (m, 2H), 4.43-4.35 (m, 1H), 4.27-4.19 | | | |
| | (m, 1H), 3.55-3.43 (m, 2H), 3.02-2.84 (m, 4H), | | | |
| | 2.81-2.71 (m, 3H), 2.63-2.59 (m, 1H), 2.55-2.52 | | | |
| | (m, 4H), 2.47-2.36 (m, 4H), 2.26-2.03 (m, 4H), 2.00-1.91 | | | |
| | (m, 2H), 1.86-1.76 (m, 3H), 1.73-1.63 (m, 2H), | | | |
| | 1.58-1.51 (m, 3H), 1.36-1.20 (m, 4H), 1.14-0.85 | | | |
| | (m, 8H), 0.80-0.74 (m, 2H) | | | |
| 20 | 1H NMR (400 MHz, DMSO-d6): δ 13.41 (s, 1H), 10.98 | 819.96 | 819.42 | 820.3 |
| | (s, 1H), 8.65 (s, 1H), 8.25-8.11 (m, 1H), 7.51 (d, J = | | | |
| | 9.1 Hz, 1H), 7.38 (s, 1H), 7.11-7.02 (m, 1H), 6.92 (d, | | | |
| | J = 7.6 Hz, 1H), 5.09-4.98 (m, 1H), 4.46-4.36 (m, | | | |
| | 1H), 4.30-4.18 (m, 1H), 3.68 (br s, 4H), 3.60-3.51 | | | |
| | (m, 1H), 3.43-3.37 (m, 2H), 3.02-2.86 (m, 3H), 2.63-2.56 | | | |
| | (m, 1H), 2.54 (s, 9H), 2.46-2.37 (m, 5H), 2.18 | | | |
| | (br d, J = 7.1 Hz, 2H), 1.99-1.87 (m, 3H), 1.79-1.66 | | | |
| | (m, 2H), 1.65-1.40 (m, 6H), 1.37-1.23 (m, 2H), 0.99-0.93 | | | |
| | (m, 2H), 0.81-0.73 (m, 2H) | | | |
| 21 | 1H NMR (400 MHz, DMSO-d6): δ 13.43 (s, 1H), 10.98 | 802.98 | 802.44 | 803.4 |
| | (s, 1H), 8.67 (s, 1H), 8.33 (s, 1H), 8.14 (s, 1H), 7.50 | | | |
| | (dd, J = 8.4, 19.6 Hz, 2H), 7.41 (s, 1H), 7.31 (d, J = | | | |
| | 8.6 Hz, 1H), 7.16 (br t, J = 7.6 Hz, 1H), 5.07 (dd, J = | | | |
| | 4.8, 13.2 Hz, 1H), 4.56-4.43 (m, 1H), 4.31 (d, J = 16.8 | | | |
| | Hz, 1H), 3.68 (br s, 4H), 3.49 (br s, 2H), 3.13-3.01 | | | |
| | (m, 2H), 2.96-2.86 (m, 1H), 2.78 (br t, J = 11.6 Hz, | | | |
| | 2H), 2.68 (s, 2H), 2.59 (br d, J = 16.4 Hz, 2H), 2.45 (br | | | |
| | s, 6H), 2.41-2.35 (m, 1H), 2.30-2.23 (m, 1H), 2.20 | | | |
| | (br d, J = 6.4 Hz, 2H), 1.98 (br dd, J = 6.0, 11.2 Hz, | | | |
| | 1H), 1.86-1.75 (m, 5H), 1.71-1.59 (m, 1H), 1.37-1.20 | | | |
| | (m, 4H), 0.96 (s, 3H), 0.49 (s, 2H), 0.35-0.24 (m, 2H) | | | |
| 22 | 1H NMR (400 MHz, DMSO-d6): 13.38 (s, 1H), 10.98 | 804.95 | 804.41 | 805.7 |
| | (s, 1H), 8.62 (s, 1H), 8.17-8.12 (m, 1H), 7.49 (dd, | | | |
| | J = 8.5, 16.0 Hz, 2H), 7.37 (s, 1H), 7.17 (t, J = 8.0 Hz, | | | |
| | 1H), 7.07 (dd, J = 2.2, 8.9 Hz, 1H), 5.07 (dd, J = 4.9, 13.3 | | | |
| | Hz, 1H), 4.52-4.39 (m, 3H), 4.36-4.27 (m, 1H), 3.63-3.47 | | | |
| | (m, 2H), 3.24 (br d, J = 14.5 Hz, 2H), 2.94 (br t, | | | |
| | J = 11.6 Hz, 5H), 2.62 (br s, 1H), 2.47-2.37 (m, 2H), | | | |
| | 1.94 (br d, J = 9.6 Hz, 5H), 1.74 (br d, J = 13.1 Hz, 4H), | | | |
| | 1.61-1.52 (m, 5H), 1.33 (br s, 1H), 1.22-1.04 (m, | | | |
| | 6H), 0.97-0.87 (m, 4H), 0.80-0.75 (m, 2H) | | | |
| 23 | 1H NMR (400 MHz, DMSO-d6): δ 13.61-13.08 (m, | 804.95 | 804.41 | 805.3 |
| | 1H), 10.99 (s, 1H), 8.63 (s, 1H), 8.23-7.98 (m, 1H), | | | |
| | 7.56-7.50 (m, 1H), 7.47 (d, J = 8.1 Hz, 1H), 7.37 (s, | | | |
| | 1H), 7.18 (t, J = 7.8 Hz, 1H), 7.13-7.05 (m, 1H), | | | |
| | 5.15-5.02 (m, 1H), 4.65-4.16 (m, 4H), 3.74-3.65 (m, 1H), | | | |
| | 3.62-3.51 (m, 1H), 3.42-3.36 (m, 2H), 3.02-2.86 | | | |
| | (m, 5H), 2.64-2.56 (m, 1H), 2.46-2.36 (m, 1H), 2.01-1.87 | | | |
| | (m, 3H), 1.83-1.67 (m, 5H), 1.64-1.53 (m, 5H), | | | |
| | 1.50-1.39 (m, 5H), 1.34-1.23 (m, 2H), 1.18-1.01 | | | |
| | (m, 4H), 1.00-0.91 (m, 2H), 0.81-0.72 (m, 2H) | | | |
| 24 | 1H NMR (400 MHz, DMSO-d6): δ 13.43 (s, 1H), 10.98 | 803.96 | 803.43 | 804.3 |
| | (s, 1H), 8.67 (s, 1H), 8.33 (s, 1H), 7.50 (dd, J = 8.4, | | | |
| | 16.0 Hz, 2H), 7.43 (s, 1H), 7.31 (d, J = 8.4 Hz, 1H), | | | |
| | 7.17 (t, J = 8.0 Hz, 1H), 5.07 (dd, J = 4.8, 13.2 Hz, 1H), | | | |
| | 4.48 (d, J = 17.2 Hz, 1H), 4.31 (d, J = 16.8 Hz, 1H), | | | |
| | 4.14-4.02 (m, 2H), 3.85-3.65 (m, 2H), 3.50 (br d, J = | | | |
| | 6.4 Hz, 2H), 3.43-3.36 (m, 2H), 3.22-3.01 (m, 2H), | | | |
| | 2.97-2.75 (m, 5H), 2.68 (s, 2H), 2.64-2.56 (m, 2H), | | | |
| | 2.47-2.35 (m, 2H), 2.02-1.80 (m, 9H), 1.74-1.62 | | | |
| | (m, 1H), 1.53-1.44 (m, 2H), 1.41-1.31 (m, 2H), 0.96 | | | |
| | (s, 3H), 0.52-0.47 (m, 2H), 0.34-0.27 (m, 2H) | | | |
| 25 | 1H NMR (400 MHz, DMSO-d6): δ 13.43 (s, 1H), 10.98 | 831.03 | 830.48 | 831.4 |
| | (s, 1H), 8.66 (s, 1H), 8.33 (s, 1H), 8.13 (s, 1H), 7.52 | | | |
| | (br d, J = 8.8 Hz, 1H), 7.47 (d, J = 8.0 Hz, 1H), 7.43-7.39 | | | |
| | (m, 1H), 7.31 (d, J = 8.8 Hz, 1H), 7.18-7.14 (m, | | | |
| | 1H), 5.07 (dd, J = 4.8, 13.2 Hz, 1H), 4.48 (br d, J = | | | |
| | 16.8 Hz, 2H), 4.30 (d, J = 16.8 Hz, 1H), 3.51-3.47 (m, | | | |
| | 2H), 2.99-2.90 (m, 4H), 2.80-2.75 (m, 2H), 2.68 (s, | | | |
| | 2H), 2.67-2.64 (m, 1H), 2.58-2.53 (m, 2H), 2.44-2.38 | | | |
| | (m, 2H), 2.24-2.11 (m, 4H), 2.01-1.95 (m, 2H),1.81 | | | |
| | (br d, J = 12.0 Hz, 4H), 1.75-1.65 (m, 2H), 1.61-1.54 | | | |
| | (m, 1H), 1.34-1.23 (m, 4H), 1.16-1.01 (m, 6H), | | | |
| | 0.96 (s, 3H), 0.89 (d, J = 6.6 Hz, 1H), 0.52-0.47 (m, | | | |
| | 2H), 0.33-0.26 (m, 2H) | | | |
| 26 | 1H NMR (400 MHz, CD3OD): δ 8.62 (s, 1H), 8.00 (s, | 831.03 | 830.48 | 831.5 |
| | 1H), 7.64 (d, J = 8.8 Hz, 1H), 7.55 (d, J = 8.4 Hz, 1H), | | | |
| | 7.53-7.49 (m, 1H), 7.44 (s, 1H), 7.20 (t, J = 8.0 Hz, | | | |
| | 1H), 5.12 (dd, J = 5.2, 13.2 Hz, 1H), 4.57-4.45 (m, | | | |
| | 2H), 3.72-3.53 (m, 4H), 3.12-3.01 (m, 2H), 2.98-2.93 | | | |
| | (m, 1H), 2.89 (br dd, J = 5.2, 13.2 Hz, 4H), 2.82 | | | |
| | (s, 2H), 2.80 (br d, J = 2.4 Hz, 1H), 2.77-2.69 (m, 1H), | | | |
| | 2.50 (br dd, J = 4.8, 13.2 Hz, 2H), 2.44-2.40 (m, 1H), | | | |
| | 2.40-2.29 (m, 3H), 2.19-2.14 (m, 1H), 2.12-2.05 | | | |
| | (m, 4H), 2.00-1.86 (m, 3H), 1.69-1.57 (m, 2H), 1.52 | | | |
| | (br s, 3H), 1.44 (br s, 3H), 1.40-1.30 (m, 3H), 1.01 (s, | | | |
| | 3H), 0.62-0.58 (m, 2H), 0.40-0.37 (m, 2H) | | | |
| 27 | 1H NMR (400 MHz, DMSO-d6) δ: 13.39 (br s, 1H), | 804.95 | 804.41 | 805.8 |
| | 10.99 (s, 1H), 8.63 (s, 1H), 8.15 (br s, 1H), 7.53-7.43 | | | |
| | (m, 2H), 7.39 (s, 1H), 7.14 (br t, J = 8.0 Hz, 1H), 7.06 | | | |
| | (br d, J = 9.0 Hz, 1H), 5.08-5.04 (m, 1H), 4.49 (br s, | | | |
| | 1H), 4.32 (br s, 1H), 4.08 (br s, 1H), 3.73 (br s, 2H), | | | |
| | 3.53-3.50 (m, 4H), 2.86 (br d, J = 4.8 Hz, 1H), 2.76 | | | |
| | (br d, J = 11.3 Hz, 2H), 2.60 (br s, 2H), 2.46-2.38 (m, | | | |
| | 1H), 2.00-1.81 (m, 7H), 1.73 (br d, J = 12.3 Hz, 4H), | | | |
| | 1.54 (s, 3H), 1.46-1.39 (m, 2H), 1.32 (br s, 4H), 1.13 | | | |
| | (br d, J = 7.4 Hz, 2H), 0.98-0.88 (m, 4H), 0.77 (br s, 2H) | | | |
| 28 | 1H NMR (400 MHz, DMSO-d6): δ 13.36-13.47 (1 H, m), | 816.00 | 815.45 | 816.4 |
| | 10.88-11.01 (1 H, m), 8.61-8.71 (1 H, m), 8.29-8.37 | | | |
| | (1 H, m), 7.47-7.56 (1 H, m), 7.27-7.43 (3 H, m), | | | |
| | 7.03-7.12 (1 H, m), 5.01-5.10 (1 H, m), 4.38-4.49 | | | |
| | (1 H, m), 4.21-4.31 (1 H, m), 3.86 (3 H, s), 3.57-3.70 | | | |
| | (5 H, m), 3.38 (3 H, dd, J = 7.27, 2.38 Hz), 3.30 | | | |
| | (4 H, s), 2.82-2.89 (2 H, m), 2.68 (3 H, s), 2.56-2.61 | | | |
| | (1 H, m), 2.43-2.45 (3 H, m), 2.12-2.15 (1 H, m), | | | |
| | 1.93-1.98 (4 H, m), 1.78-1.85 (2 H, m), 1.48-1.63 | | | |
| | (3 H, m), 1.13-1.25 (2 H, m), 0.87-0.96 (5 H, m), | | | |
| | 0.45-0.52 (2 H, m), 0.25-0.33 (2 H, m) | | | |
| 29 | 1H NMR (400 MHz, DMSO-d6): δ 13.38 (s, 1H), 10.98 | 804.95 | 804.41 | 805.8 |
| | (s, 1H), 8.63 (s, 1H), 8.21-8.10 (m, 1H), 7.55-7.42 | | | |
| | (m, 2H), 7.40 (s, 1H), 7.15 (t, J = 7.9 Hz, 1H), 7.09-6.99 | | | |
| | (m, 1H), 5.12-5.01 (m, 1H), 4.48 (d, J = 16.8 Hz, | | | |
| | 1H), 4.30 (d, J = 16.9 Hz, 1H), 4.13-3.98 (m, 2H), | | | |
| | 3.73-3.60 (m, 2H), 3.54-3.36 (m, 4H), 2.97-2.84 | | | |
| | (m, 1H), 2.76 (br t, J = 11.6 Hz, 2H), 2.63-2.55 (m, | | | |
| | 1H), 2.47-2.35 (m, 2H), 2.02-1.92 (m, 1H), 1.91-1.81 | | | |
| | (m, 2H), 1.78-1.66 (m, 4H), 1.55 (s, 3H), 1.50-1.41 | | | |
| | (m, 7H), 1.34-1.22 (m, 4H), 1.20-1.12 (m, 2H), | | | |
| | 1.00-0.89 (m, 2H), 0.82-0.72 (m, 2H) | | | |
| 30 | 1H NMR (400 MHz, DMSO-d6): δ 13.45 (1 H, s), | 803.96 | 803.43 | 804.3 |
| | 10.92-11.04 (1 H, m), 8.61-8.72 (1 H, m), 8.31-8.35 | | | |
| | (1 H, m), 7.52 (1 H, d, J = 8.93 Hz), 7.46 (1 H, d, J = 8.31 | | | |
| | Hz), 7.40 (1 H, s), 7.29-7.34 (1 H, m), 7.14-7.19 (1 | | | |
| | H, m), 5.07 (1 H, dd, J = 13.02, 5.07 Hz), 4.45-4.51 | | | |
| | (1 H, m), 4.32 (1 H, s), 3.65-3.70 (4 H, m), 2.85-2.99 | | | |
| | (4 H, m), 2.67-2.68 (3 H, m), 2.55-2.62 (2 H, m), | | | |
| | 2.40-2.43 (5 H, m), 2.11-2.14 (2 H, m), 1.90-1.98 | | | |
| | (6 H, m), 1.78-1.84 (2 H, m), 1.50-1.59 (3 H, m), | | | |
| | 1.14-1.21 (2 H, m), 0.90-0.96 (5 H, m), 0.48-0.51 | | | |
| | (2 H, m), 0.28-0.31 (2 H, m) | | | |

Exemplary assay for testing LRRK2 degradation driven by exemplary compounds designed to target LRRK2.

The assay measures the degradation of wildtype and G2019S LRRK2 tagged with a HiBit tag on the C-terminus of the protein that was expressed from a mammalian expression vector, driven by the ubiquitin promoter in HEK293 cells. Each compound dose-response was repeated on two separate days, on three separate plates each day.

Plasmid Preparation. Transfection mixes were assembled as follows and incubated for 30 minutes at room temperature. In a 15 mL tube, 5.25 mL Opti-MEM (no additions) was mixed with 17 μL Firefly Luciferase plasmid at 1 μg/μL and 158 μL WT plasmid DNA at 1 μg/μL (175 μg total DNA) were mixed by flicking. In a new 15 mL tube, 5.25 mL OptiMEM was mixed with 17 μL Firefly Luciferase plasmid at 1 μg/μL and 158 μL G2019S plasmid DNA at 1 μg/μL (175 μg total DNA) were mixed by flicking. X-tremeGene HP was mixed thoroughly using a vortex. Next, 175 μL was added to each tube and flicked to mix. Both tubes were left to incubate for 30 minutes at room temperature.

While the transfection mixes were incubating, HEK293 cells (acquired from ATCC; ATCC CRL-1573) were harvested with trypsin. Once cells are detached, the cells were resuspended in 12 mL OptiMEM+5% FBS and transferred to a 50 mL tube. The cells were mixed well and counted. Using OptiMEM+5% FBS, the cells were diluted in two 250 mL conical tubes at 0.71×10⁶ cells/mL in 70 mL. One tube was labeled “WT” and the other “G2019S”. The WT and G2019S transfection mixes were added dropwise to the corresponding 250 mL tubes. The tubes were mixed first by pipetting then by swirling. The tubes were incubated at room temperature for at least 5 minutes.

Each tube was swirled before dispensing and after every three plates. Seventy microliters of cells were dispensed with WT or G2019S DNA to seven plates each. Three plates of each were tested with compound plate one (preparation described below) and three plates of each were tested with compound plate two (preparation described below). The first plate from each set served as a “prime” plate and was not used to test compounds. Each plate was incubated in the hood for 10 minutes before placing in the 37° C. incubator for 24 hours.

Preparation of Compound and Assay Plates. Two compound plates were made using 96 well polypropylene plates. Compounds were made up at 10 mM and were diluted to 1 mM in 30 μL. Each dose response curve included a well of DMSO, as a negative control and for normalization, and a well of 0.5 μM of Compound 4 as a positive control. In addition to seven test compounds, each plate also included a dose response of Compound 4. The compound plates were spun down along at 1200 rpm for 2 minutes.

The two compound plates were then mixed and 2 μL was diluted in intermediate plates having 248 μL of Opti-Mem in each well. Next, 10 μL diluted compounds from the intermediate plates were added to each test plate (three WT and three G2019S plates per compound plate for a total of 12 assay plates). The plates were incubated for 24 hours at 37° C.

All assay plates and all Nano-Glo Dual-Luciferase Reporter Assay System components (except for the DLR substrate) were equilibrated to room temperature. Next, the luciferase buffer was mixed with the lyophilized amber bottle until fully dissolved, and 75 μL of the luciferase mixture was added to each well of each assay plate. The assay plates were incubated for 10 minutes at room temperature with shaking for at least 5 minutes, and then read on a plate reader.

Developing Plates and Analyzing Data. One milliliter of DLR substrate and 1 mL LgBiT Protein were added to the Stop and Glo buffer, and 75 μL of the mixture was added to each well of each plate. Optically clear seals were added to each plate and each plate was incubated for 20 minutes with shaking for at least 10 minutes, and then read on a plate reader.

As mentioned above, plates were run in triplicate and assay repeated twice (total of 6 replicates per exemplary compound). Each cell was examined for firefly luciferase for cell number and viability and Nanoluc for the LRRK2-HiBit quantification.

Ratio of (HiBit/luciferase)*1000 was determined and the data was normalized to % of DMSO median value. Curve fitting was performed on each individual plate. The data for exemplary compounds of Table 1 below is shown below in Table 2 in the *G2019S DC50 (nM), **G2019S Dmax (%), *WT DC50 (nM), and **WT Dmax (%) columns.

### Exemplary Assay for Testing LRRK2 Degradation Driven by Exemplary Compounds Designed to Target LRRK2

The assay measures the degradation of LRRK2 in cells where the C-terminus (3′) of the endogenous gene has been tagged with a HiBit sequence in HEK293 cells. The cells also express firefly lucisferase, expressed from a Cytomegalovirus promoter and introduced into the HiBit tagged cells and stably expressed. The Nano-Glo® Dual Luciferase Reporter Assay System (Promega™, Madison, WI) was utilized.

Day 1—Preparation of Compound and Assay Plates. Two sets of plates were prepared: a triplicate set for the HiBit assay in white 384-well plates and a triplicate set of plate in black 384-well plates for the Alamar Blue cell viability assay. Briefly, the growth media (DMEM+Glutamax-10% fetal bovine serum-1% Penicillin-Streptomicin) from two T128 flasks was aspirated from the flasks. Cells were washed with Dulbecco's Phosphate Buffered Saline (dPBS) and aspirated. Trypsin (3 mL per flask) was added and the flasks were incubated for 2-3 minutes.

Ten mL of OptiMEM-10% fetal bovine-1% penicillin-streptomycin (hereinafter, “OptiMEM media”) was added to the flask and the cells and transferred to a 50 mL conical tube. A cell count (25 ul of cell into Effendorf vial+25 ul of Trypan Blue Stain) was performed and the cell density adjusted to 15,000 cell/45 μl/well (3.33×10\hat{ }5/mL) in OptiMEM media.

Forty-five microliters of the cell suspension (15,000 cells) was aliquoted to each well of the white 384-well plate. The plates incubated at room temperature for 10 minutes before being placed in the 37° C.+5% CO₂ incubator overnight

Day 2—Compound Treatment. Exemplary compounds were prepared at a 1 mM starting concentration and 1:3 serial dilution for 11 points CRC prepared and stored in the freezer. The Master Compound Plate was thawed overnight at room temperature. DMSO (20 μL) was added into column 24 of the Master Compound Plate for negative control and 20 μL of 300 μM of Compound 4 in column 23 as positive control.

Intermediate Compound Plate with 4% DMSO in OptiMEM Media. DMSO was added to warm OptiMEM media to achieve a 4% DMSO solution (approximately 50 mL/plate). One-hundred microliters of the OptiMEM-4% DMSO was aliquoted to each well of 384-Well Deep Well Microplates.

The Master Compound Plate and the Intermediate Compound Plate were spun down.

One microliter of compound from the Master Compound Plate was transferred into the Intermediate plate (a 1:100 dilution). The diluted mixture was mixed and 5 μL transferred into the assay plate (a 1:10 dilution) for the final starting concentration of 1 μM. The Treated Assay plates were incubated for 24 hours at 37° C.+5% CO₂. The Master Compound Plate was sealed and store at room temperature for a second run that was performed within a week.

Day 3—HiBit Assay. Five microliters of Alamar Blue was added to each well of the black 384-well plates. The plates were incubated for 2 hours in the incubator (37° C.+5% CO₂) and at room temperature for one hour. Fluorescence of each plate was read on a plated reader for the Alamar Blue viability assay.

One set of white assay plates was warmed to room temperature (45 minute).

The One Glo luciferase mixture was prepared. The media from white 384-well assay plates was aspirated. Twenty-five μL of the One Glo luciferase mixture was added to each well of the assay plates. The plates were incubated on the bench (room temperature) for 45 minutes, including 10 minutes of shaking at 700 rpm. The luminescence of each plate was read on a plate reader.

1:100 DLR substrate and 1:100 LgBiT Protein dilution were added to the Promega Stop and Glo buffer and mixed just before addition to assay plates. Twenty-five microliters of Stop and Glo mixture was added to each well. Assay plates incubated for at least 45 minutes, including 10 minutes of shaking at 700 rpm. The luminescence of each plate was read on a plate reader.

Analysis of LRRK2 HiBit Screening assays. As mentioned above, plates were run in triplicate and the assay repeated twice (total of 6 replicate for exemplary compounds). For each treatment, measurements were taken for firefly luciferase for cell number, cell viability (Alamar Blue), and Nanoluc for the LRRK2-HiBit quantification.

The LRRK2 HiBit and alamar blue signal was normalized to % DMSO median value for each plate. Curve fitting was performed on each compound for replicates across three plates.

Data for the compounds disclosed herein is provided in Table 2.

TABLE 2

| Ex. | WT LRRK2 *IC₅₀ (nM) | WT LRRK2 **Dmax (%) |
|---|---|---|
| 1 | A | B |
| 2 | A | B |
| 3 | A | B |
| 4 | A | B |
| 5 | A | B |
| 6 | A | B |
| 7 | A | B |
| 8 | A | B |
| 9 | A | B |
| 10 | A | B |
| 11 | A | B |
| 12 | A | B |
| 13 | A | B |
| 14 | A | B |
| 15 | A | B |
| 16 | A | B |
| 17 | A | B |
| 18 | A | A |
| 19 | A | B |
| 20 | A | C |
| 21 | B | B |
| 22 | B | A |
| 23 | B | B |
| 24 | B | C |
| 25 | B | C |
| 26 | C | B |
| 27 | C | B |
| 28 | C | B |
| 29 | C | A |
| 30 | D | C |

*IC₅₀ Ranges: A < 10; 10 ≤ B < 50; 50 ≤ C < 100; D ≥ 100
**D_{Max} Ranges: A ≥ 70; 50 ≤ B < 70; C < 50

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound selected from: 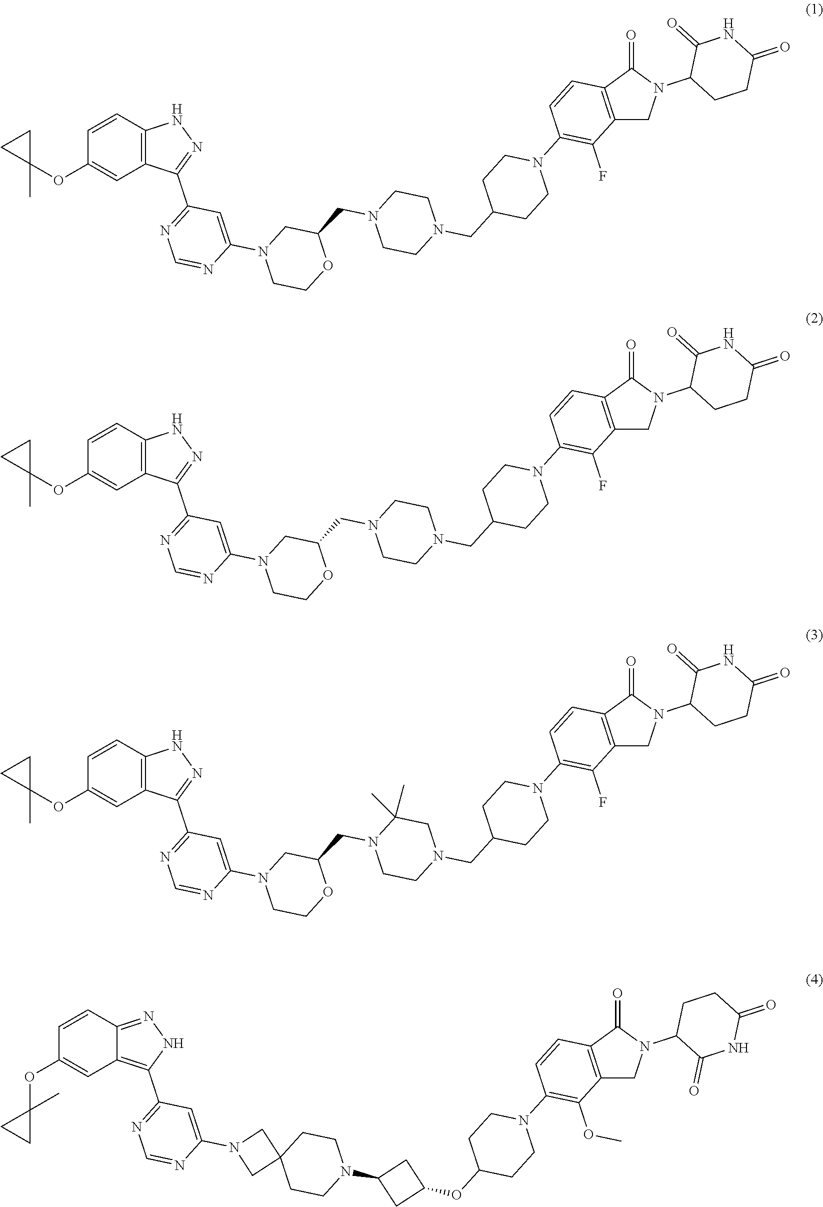 [SMILES: CC1(Oc2ccc3[nH]nc(-c4cc(N5CCO[C@H](CN6CCN(CC7CCN(c8ccc9c(c8F)CN(C8CCC(=O)NC8=O)C9=O)CC7)CC6)C5)ncn4)c3c2)CC1 ; CC1(Oc2ccc3[nH]nc(-c4cc(N5CCO[C@@H](CN6CCN(CC7CCN(c8ccc9c(c8F)CN(C8CCC(=O)NC8=O)C9=O)CC7)CC6)C5)ncn4)c3c2)CC1 ; CC1(Oc2ccc3[nH]nc(-c4cc(N5CCO[C@H](CN6CCN(CC7CCN(c8ccc9c(c8F)CN(C8CCC(=O)NC8=O)C9=O)CC7)CC6(C)C)C5)ncn4)c3c2)CC1 ; COc1c(N2CCC(O[C@H]3C[C@H](N4CCC5(CC4)CN(c4cc(-c6[nH]nc7ccc(OC8(C)CC8)cc67)ncn4)C5)C3)CC2)ccc2c1CN(C1CCC(=O)NC1=O)C2=O] 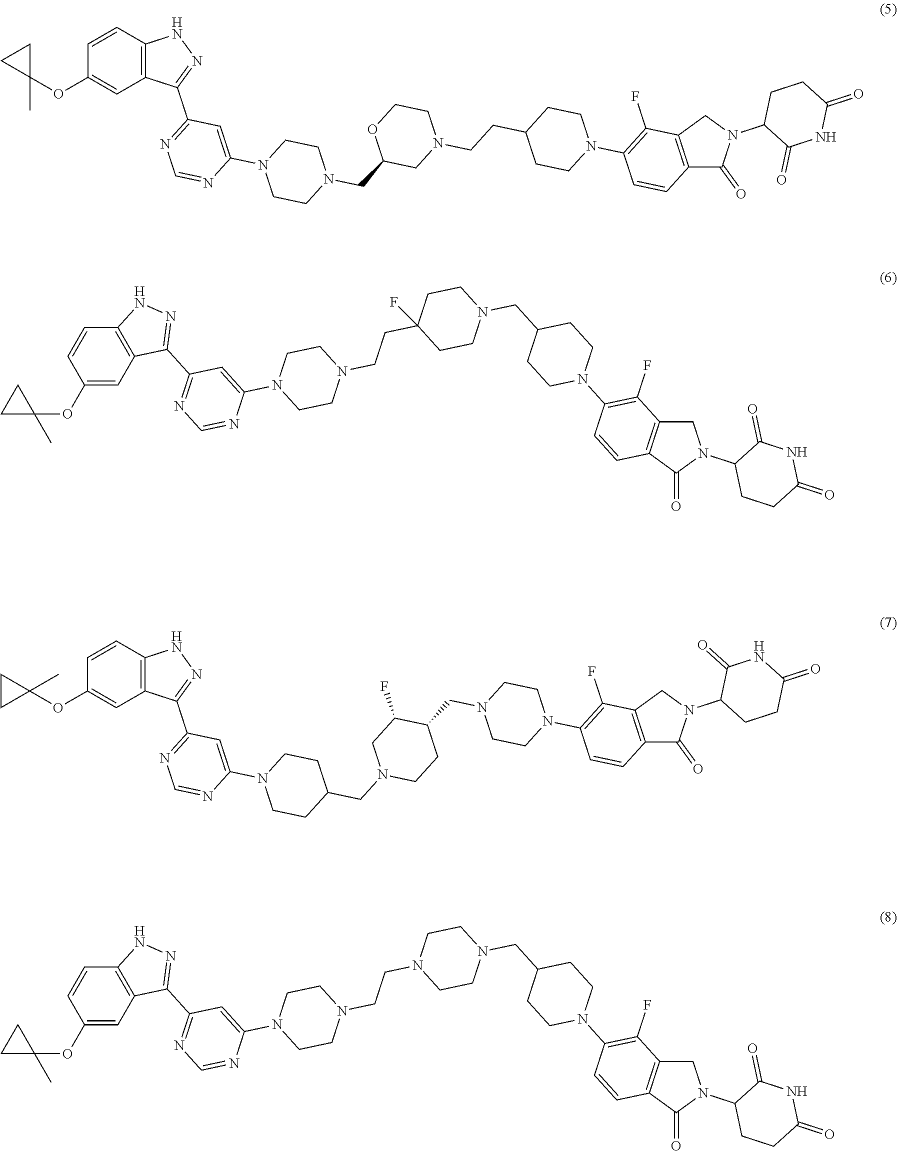 [SMILES: CC1(Oc2ccc3[nH]nc(-c4cc(N5CCN(C[C@@H]6CN(CCC7CCN(c8ccc9c(c8F)CN(C8CCC(=O)NC8=O)C9=O)CC7)CCO6)CC5)ncn4)c3c2)CC1 ; CC1(Oc2ccc3[nH]nc(-c4cc(N5CCN(CCC6(F)CCN(CC7CCN(c8ccc9c(c8F)CN(C8CCC(=O)NC8=O)C9=O)CC7)CC6)CC5)ncn4)c3c2)CC1 ; CC1(Oc2ccc3[nH]nc(-c4cc(N5CCC(CN6CC[C@@H](CN7CCN(c8ccc9c(c8F)CN(C8CCC(=O)NC8=O)C9=O)CC7)[C@@H](F)C6)CC5)ncn4)c3c2)CC1 ; CC1(Oc2ccc3[nH]nc(-c4cc(N5CCN(CCN6CCN(CC7CCN(c8ccc9c(c8F)CN(C8CCC(=O)NC8=O)C9=O)CC7)CC6)CC5)ncn4)c3c2)CC1] 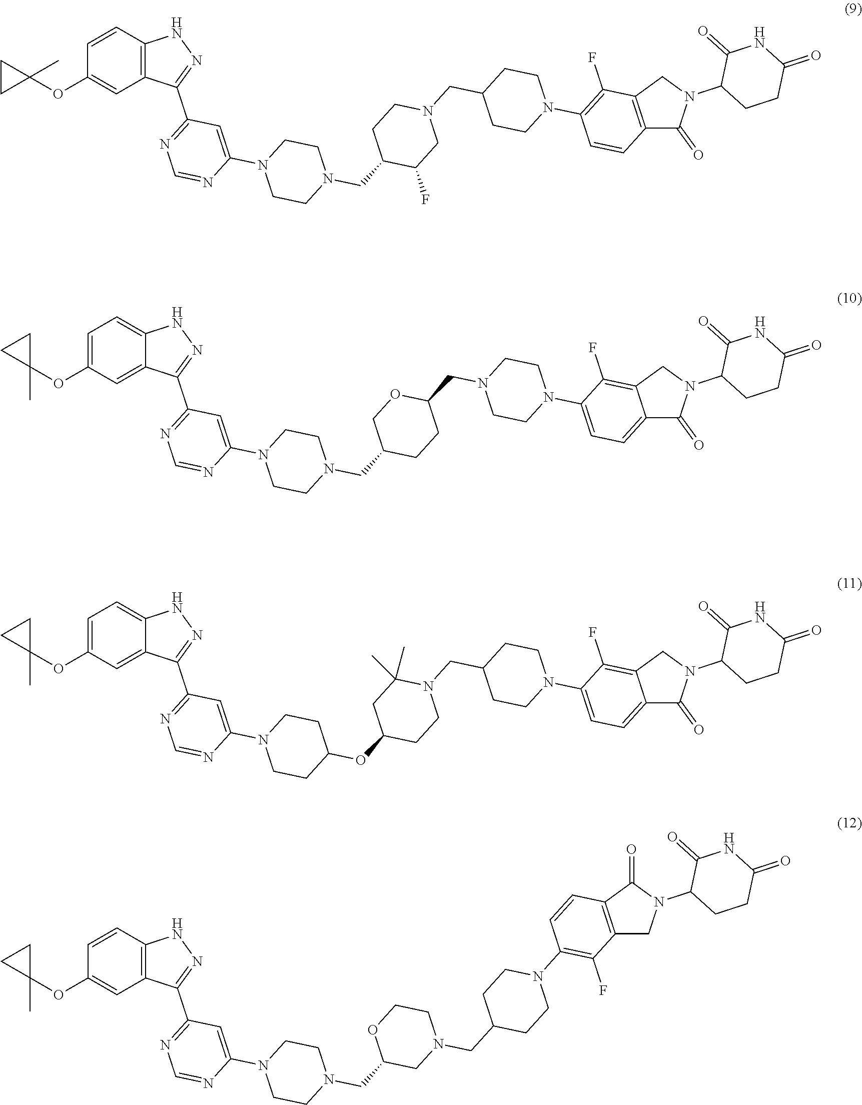 [SMILES: CC1(Oc2ccc3[nH]nc(-c4cc(N5CCN(C[C@@H]6CCN(CC7CCN(c8ccc9c(c8F)CN(C8CCC(=O)NC8=O)C9=O)CC7)C[C@@H]6F)CC5)ncn4)c3c2)CC1 ; CC1(Oc2ccc3[nH]nc(-c4cc(N5CCN(C[C@H]6CC[C@H](CN7CCN(c8ccc9c(c8F)CN(C8CCC(=O)NC8=O)C9=O)CC7)OC6)CC5)ncn4)c3c2)CC1 ; CC1(Oc2ccc3[nH]nc(-c4cc(N5CCC(O[C@@H]6CCN(CC7CCN(c8ccc9c(c8F)CN(C8CCC(=O)NC8=O)C9=O)CC7)C(C)(C)C6)CC5)ncn4)c3c2)CC1 ; CC1(Oc2ccc3[nH]nc(-c4cc(N5CCN(C[C@H]6CN(CC7CCN(c8ccc9c(c8F)CN(C8CCC(=O)NC8=O)C9=O)CC7)CCO6)CC5)ncn4)c3c2)CC1] 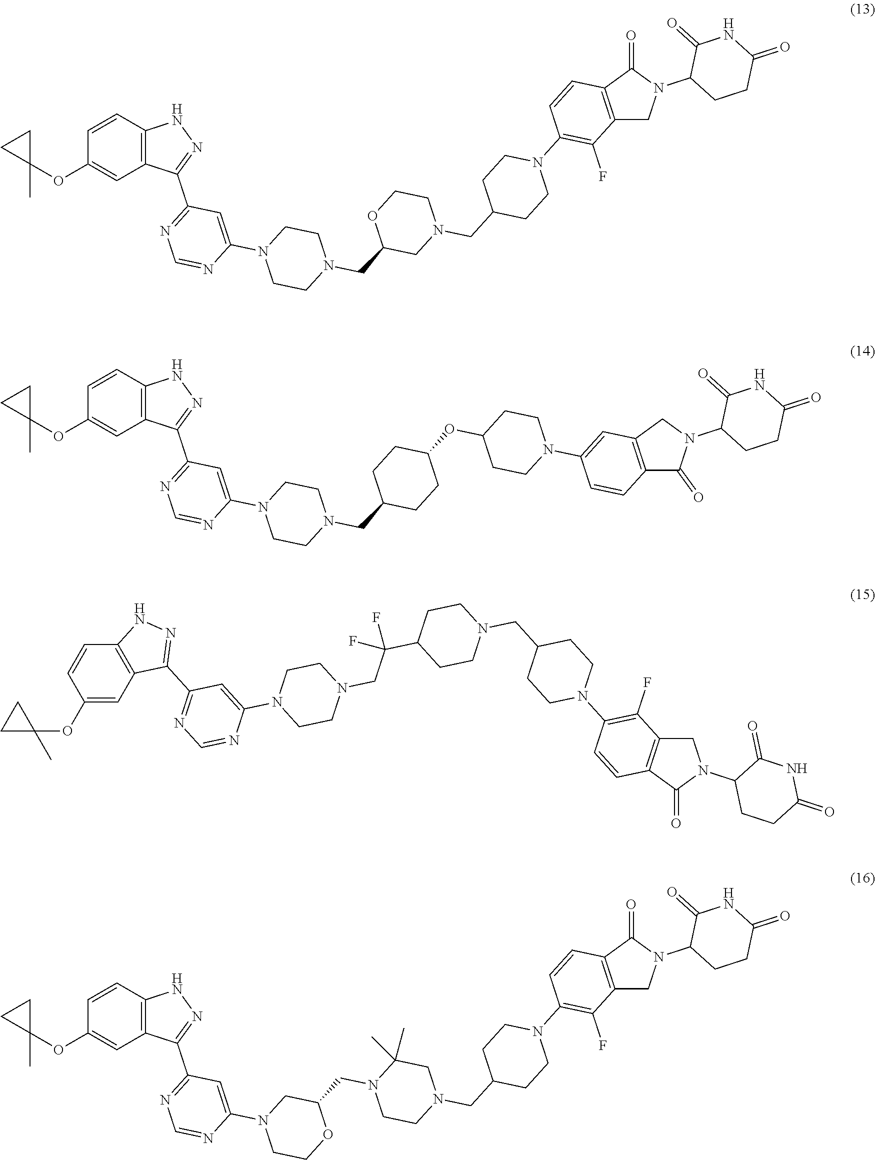 [SMILES: CC1(Oc2ccc3[nH]nc(-c4cc(N5CCN(C[C@@H]6CN(CC7CCN(c8ccc9c(c8F)CN(C8CCC(=O)NC8=O)C9=O)CC7)CCO6)CC5)ncn4)c3c2)CC1 ; CC1(Oc2ccc3[nH]nc(-c4cc(N5CCN(C[C@H]6CC[C@H](OC7CCN(c8ccc9c(c8)CN(C8CCC(=O)NC8=O)C9=O)CC7)CC6)CC5)ncn4)c3c2)CC1 ; CC1(Oc2ccc3[nH]nc(-c4cc(N5CCN(CC(F)(F)C6CCN(CC7CCN(c8ccc9c(c8F)CN(C8CCC(=O)NC8=O)C9=O)CC7)CC6)CC5)ncn4)c3c2)CC1 ; CC1(Oc2ccc3[nH]nc(-c4cc(N5CCO[C@@H](CN6CCN(CC7CCN(c8ccc9c(c8F)CN(C8CCC(=O)NC8=O)C9=O)CC7)CC6(C)C)C5)ncn4)c3c2)CC1] 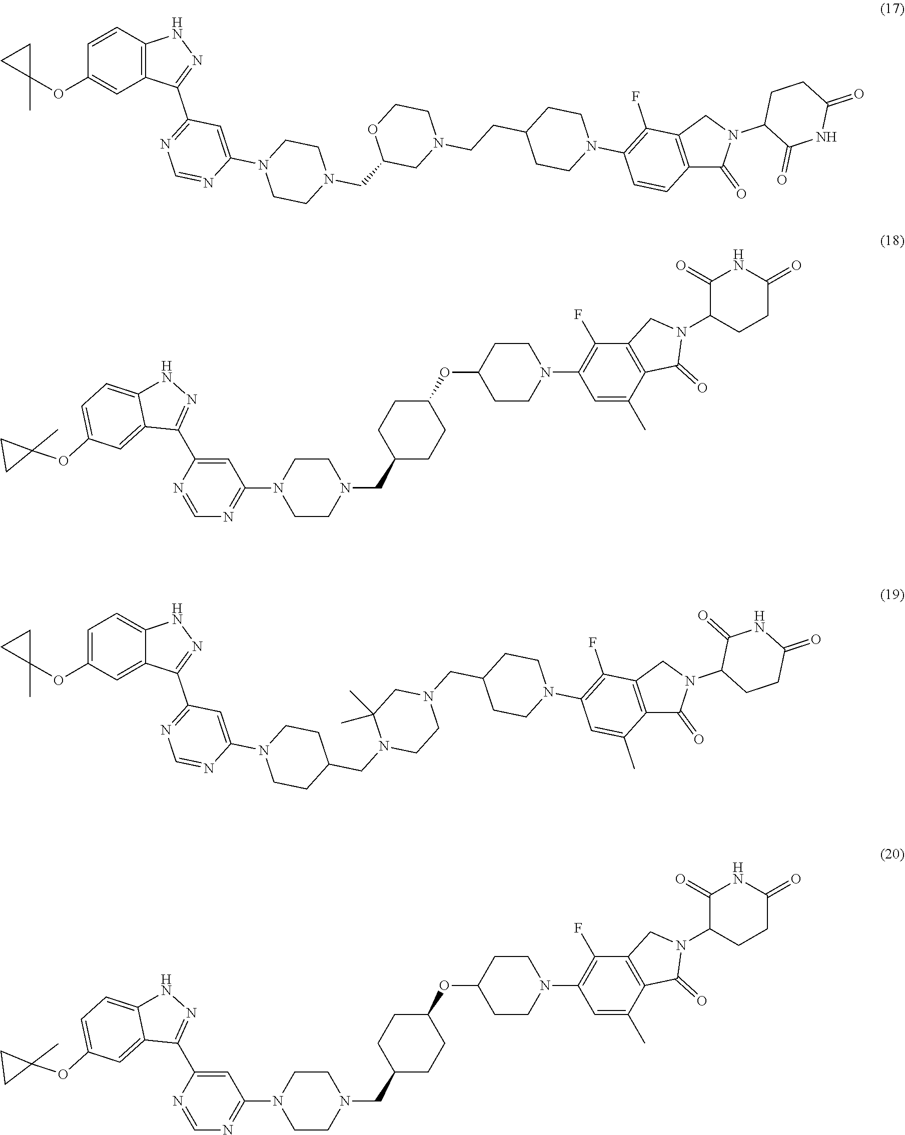 [SMILES: CC1(Oc2ccc3[nH]nc(-c4cc(N5CCN(C[C@H]6CN(CCC7CCN(c8ccc9c(c8F)CN(C8CCC(=O)NC8=O)C9=O)CC7)CCO6)CC5)ncn4)c3c2)CC1 ; Cc1cc(N2CCC(O[C@H]3CC[C@H](CN4CCN(c5cc(-c6n[nH]c7ccc(OC8(C)CC8)cc67)ncn5)CC4)CC3)CC2)c(F)c2c1C(=O)N(C1CCC(=O)NC1=O)C2 ; Cc1cc(N2CCC(CN3CCN(CC4CCN(c5cc(-c6n[nH]c7ccc(OC8(C)CC8)cc67)ncn5)CC4)C(C)(C)C3)CC2)c(F)c2c1C(=O)N(C1CCC(=O)NC1=O)C2 ; Cc1cc(N2CCC(O[C@H]3CC[C@@H](CN4CCN(c5cc(-c6n[nH]c7ccc(OC8(C)CC8)cc67)ncn5)CC4)CC3)CC2)c(F)c2c1C(=O)N(C1CCC(=O)NC1=O)C2] 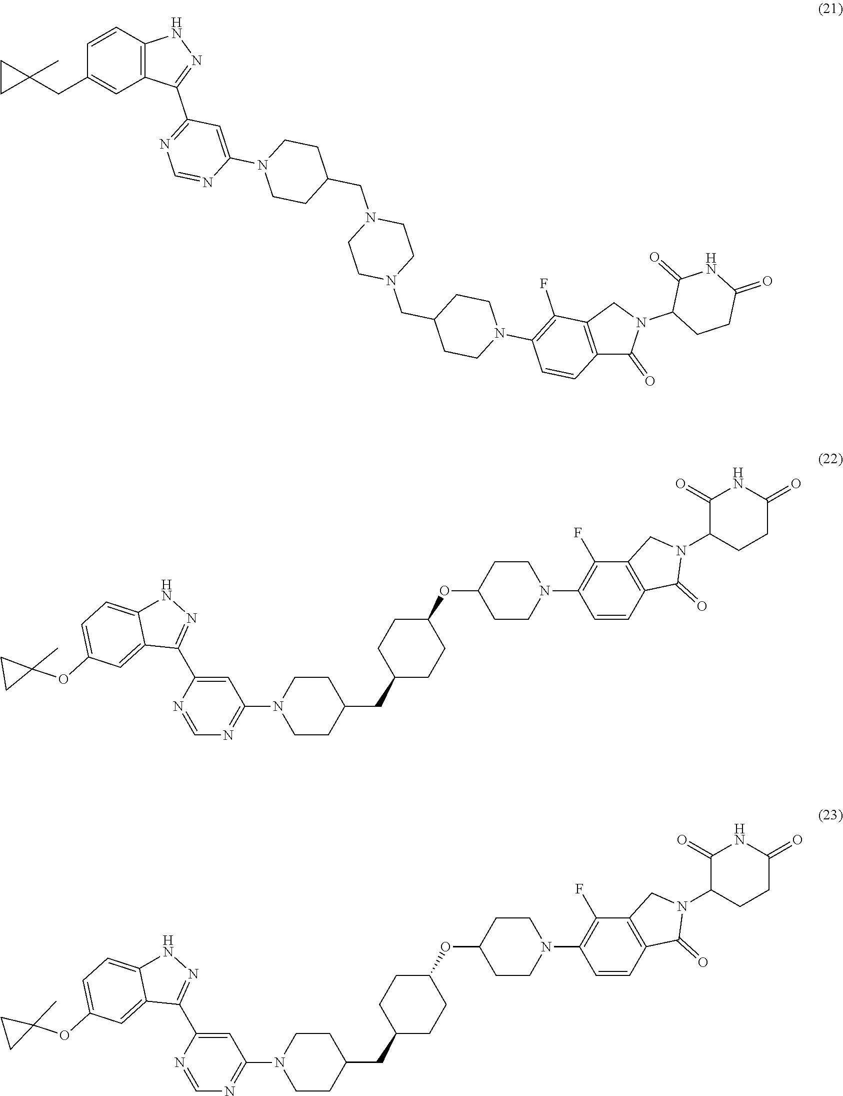 [SMILES: CC1(Cc2ccc3[nH]nc(-c4cc(N5CCC(CN6CCN(CC7CCN(c8ccc9c(c8F)CN(C8CCC(=O)NC8=O)C9=O)CC7)CC6)CC5)ncn4)c3c2)CC1 ; CC1(Oc2ccc3[nH]nc(-c4cc(N5CCC(C[C@H]6CC[C@@H](OC7CCN(c8ccc9c(c8F)CN(C8CCC(=O)NC8=O)C9=O)CC7)CC6)CC5)ncn4)c3c2)CC1 ; CC1(Oc2ccc3[nH]nc(-c4cc(N5CCC(C[C@H]6CC[C@H](OC7CCN(c8ccc9c(c8F)CN(C8CCC(=O)NC8=O)C9=O)CC7)CC6)CC5)ncn4)c3c2)CC1] 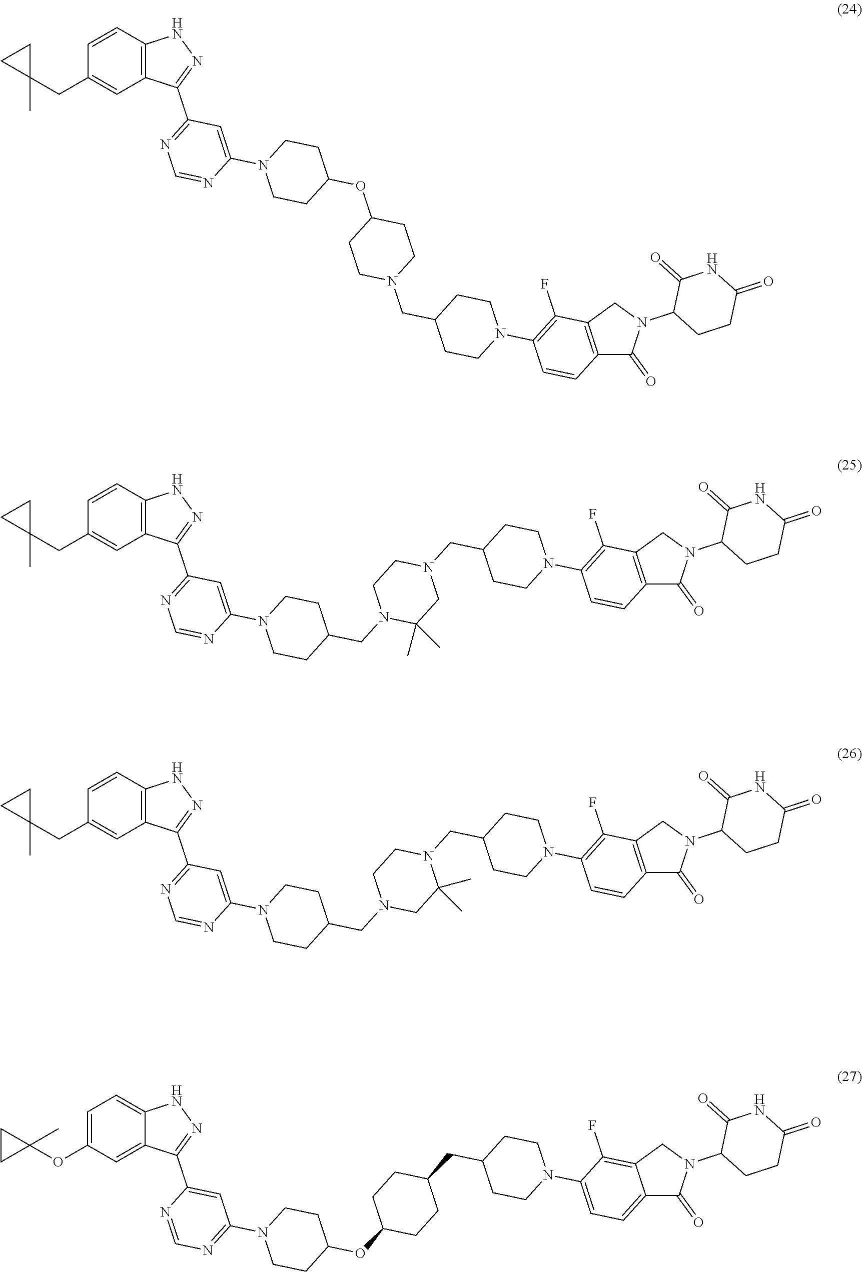 [SMILES: CC1(Cc2ccc3[nH]nc(-c4cc(N5CCC(OC6CCN(CC7CCN(c8ccc9c(c8F)CN(C8CCC(=O)NC8=O)C9=O)CC7)CC6)CC5)ncn4)c3c2)CC1 ; CC1(Cc2ccc3[nH]nc(-c4cc(N5CCC(CN6CCN(CC7CCN(c8ccc9c(c8F)CN(C8CCC(=O)NC8=O)C9=O)CC7)CC6(C)C)CC5)ncn4)c3c2)CC1 ; CC1(Cc2ccc3[nH]nc(-c4cc(N5CCC(CN6CCN(CC7CCN(c8ccc9c(c8F)CN(C8CCC(=O)NC8=O)C9=O)CC7)C(C)(C)C6)CC5)ncn4)c3c2)CC1 ; CC1(Oc2ccc3[nH]nc(-c4cc(N5CCC(O[C@H]6CC[C@@H](CC7CCN(c8ccc9c(c8F)CN(C8CCC(=O)NC8=O)C9=O)CC7)CC6)CC5)ncn4)c3c2)CC1] 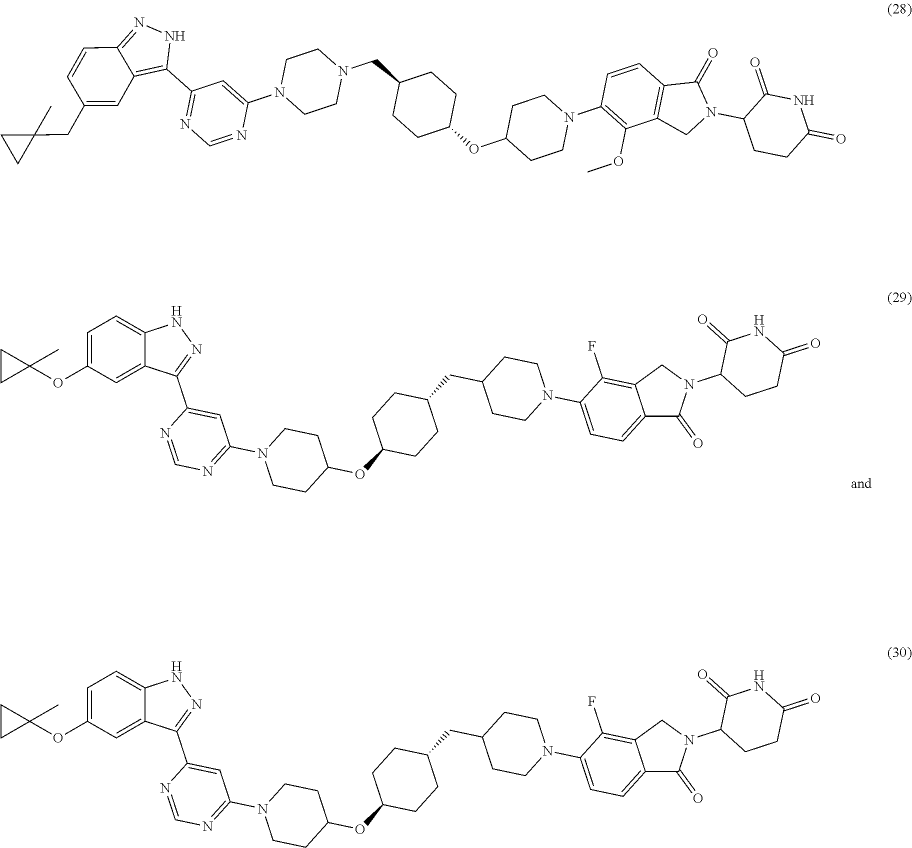 [SMILES: COc1c(N2CCC(O[C@H]3CC[C@H](CN4CCN(c5cc(-c6[nH]nc7ccc(CC8(C)CC8)cc67)ncn5)CC4)CC3)CC2)ccc2c1CN(C1CCC(=O)NC1=O)C2=O ; CC1(Oc2ccc3[nH]nc(-c4cc(N5CCC(O[C@H]6CC[C@H](CC7CCN(c8ccc9c(c8F)CN(C8CCC(=O)NC8=O)C9=O)CC7)CC6)CC5)ncn4)c3c2)CC1 ; CC1(Oc2ccc3[nH]nc(-c4cc(N5CCC(O[C@H]6CC[C@H](CC7CCN(c8ccc9c(c8F)CN(C8CCC(=O)NC8=O)C9=O)CC7)CC6)CC5)ncn4)c3c2)CC1]
or a pharmaceutically acceptable salt thereof.

2. The compound of claim 1, wherein the compound is selected from: 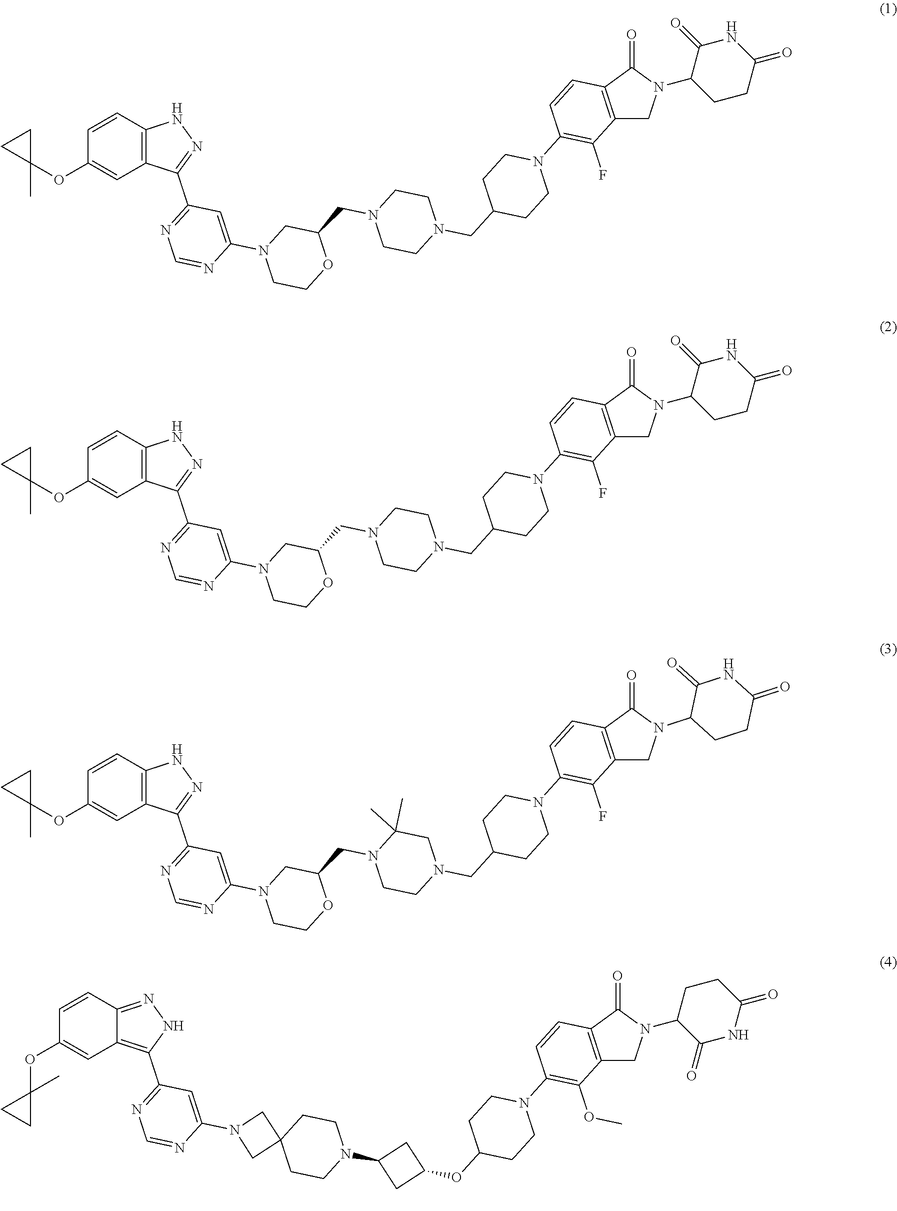 [SMILES: CC1(Oc2ccc3[nH]nc(-c4cc(N5CCO[C@H](CN6CCN(CC7CCN(c8ccc9c(c8F)CN(C8CCC(=O)NC8=O)C9=O)CC7)CC6)C5)ncn4)c3c2)CC1 ; CC1(Oc2ccc3[nH]nc(-c4cc(N5CCO[C@@H](CN6CCN(CC7CCN(c8ccc9c(c8F)CN(C8CCC(=O)NC8=O)C9=O)CC7)CC6)C5)ncn4)c3c2)CC1 ; CC1(Oc2ccc3[nH]nc(-c4cc(N5CCO[C@H](CN6CCN(CC7CCN(c8ccc9c(c8F)CN(C8CCC(=O)NC8=O)C9=O)CC7)CC6(C)C)C5)ncn4)c3c2)CC1 ; COc1c(N2CCC(O[C@H]3C[C@H](N4CCC5(CC4)CN(c4cc(-c6[nH]nc7ccc(OC8(C)CC8)cc67)ncn4)C5)C3)CC2)ccc2c1CN(C1CCC(=O)NC1=O)C2=O] 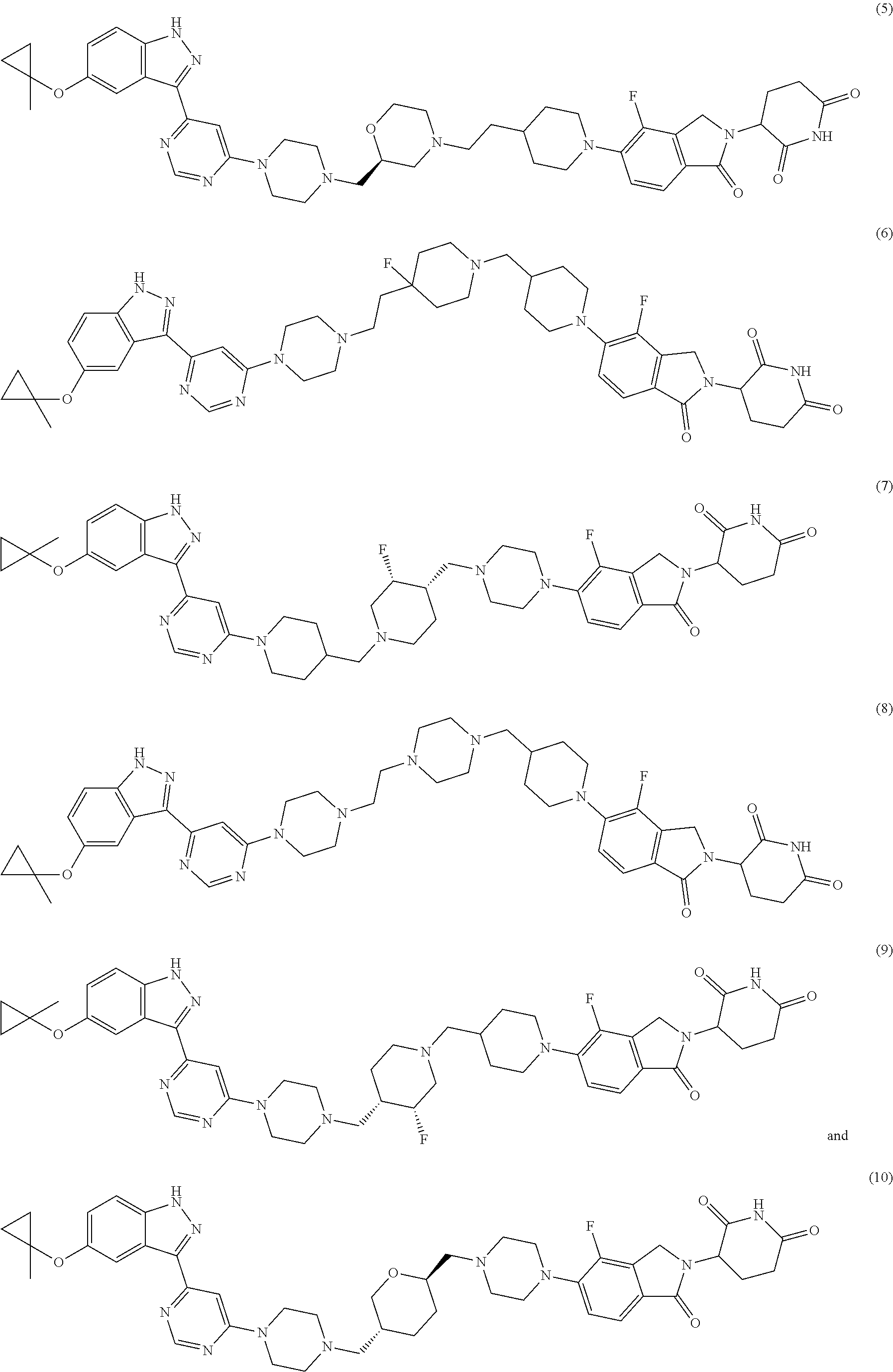
or a pharmaceutically acceptable salt thereof.

3. The compound of claim 1, wherein the compound is selected from: 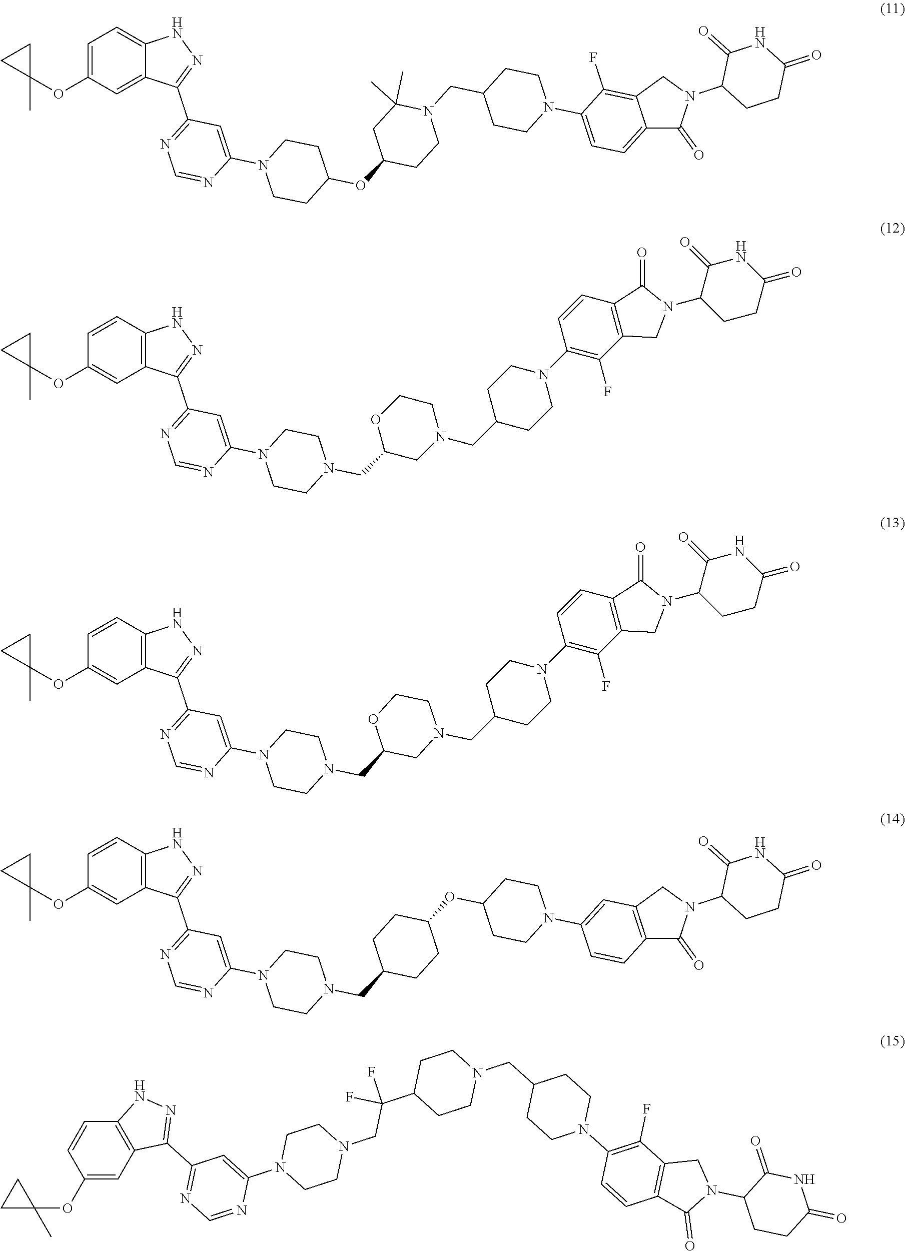 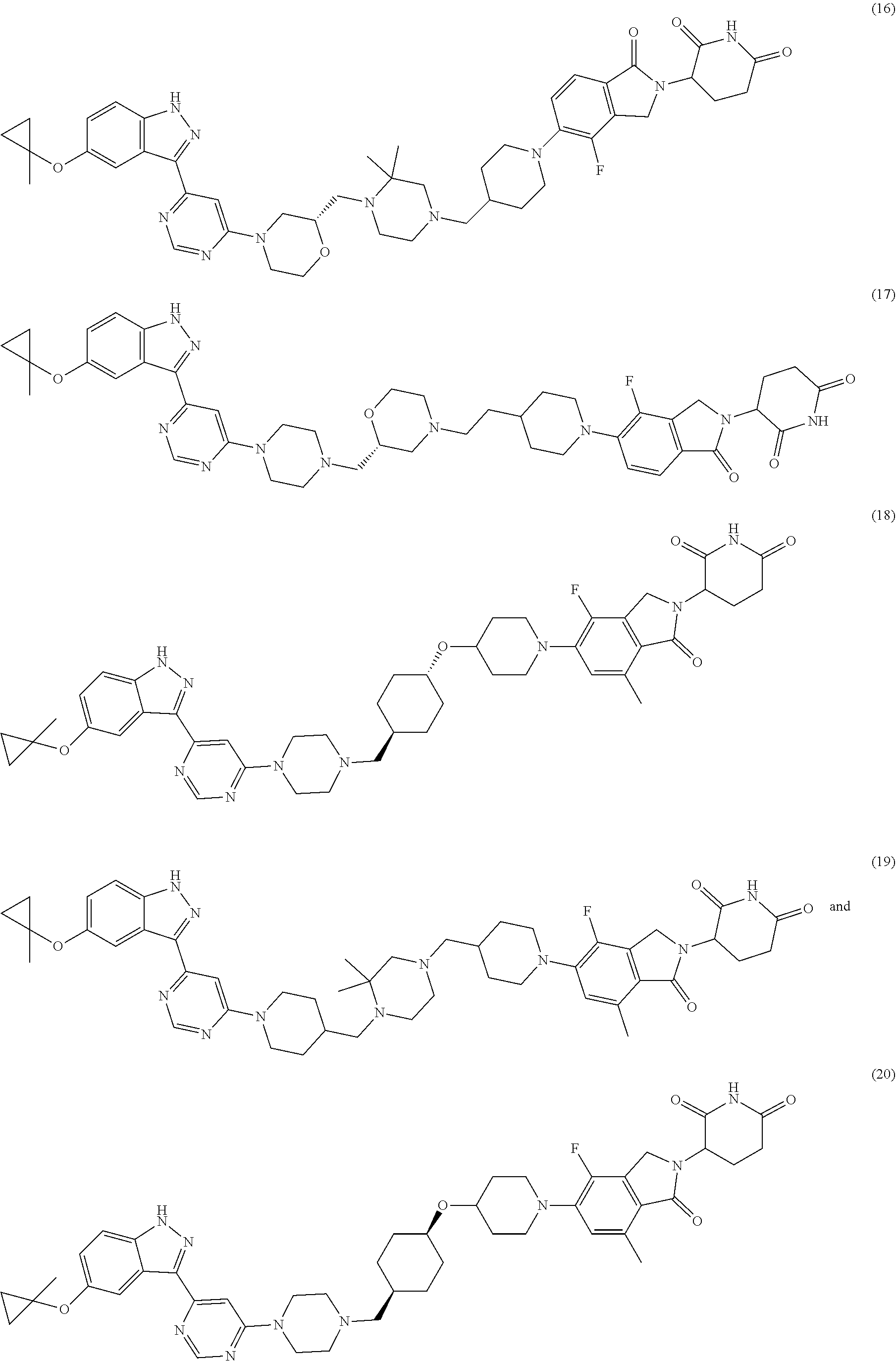
or a pharmaceutically acceptable salt thereof.

4. A pharmaceutical composition comprising a compound of any one of claims 1-3, or a pharmaceutically acceptable salt thereof, and a pharmaceutically acceptable carrier.

5. The pharmaceutical composition of claim 4, wherein the pharmaceutical composition further comprises additional bioactive agent.

6. The pharmaceutical composition of claim 5, wherein the additional bioactive agent is an anti-inflammatory, a chemotherapy agent, or an immunomodulatory agent.

7. A method of treating a disease, a disorder, or a symptom causally related to LRRK2 in a subject in need thereof comprising administering to the subject an effective amount of a compound of any one of claims 1-3, or the pharmaceutical composition of claim 4.

8. The method of claim 7, wherein the disease or disorder is Parkinson's disease (PD), LRRK2 mutation associated PD, primary tauopathies, lewy body dementia, Crohn's Disease, Leprosy, neuroinflammation, Progressive Supranuclear Palsy, Picks disease, FTDtau, TDP-43 Frontal Temporal Dementia, TDP-43 ALS, c9orf ALS, Huntington's disease, spinocerebellar ataxias (SCAs) 1, 2, 3, 6, 7 and 17, dentatorubral pallidoluysian atrophy (DRPLA), systemic lupus erythematosus (SLE), or Kennedy's disease.

9. The method of claim 7 or 8, wherein the disease or disorder is Parkinson's disease (PD), LRRK2 mutation associated PD, primary tauopathies, lewy body dementia, Crohn's Disease, Leprosy, neuroinflammation, Progressive Supranuclear Palsy, Picks disease, FTDtau, TDP-43 Frontal Temporal Dementia, TDP-43 ALS, c9orf ALS, Huntington's disease, spinocerebellar ataxias (SCAs) 1, 2, 3, 6, 7 and 17, dentatorubral pallidoluysian atrophy (DRPLA), or Kennedy's disease.

10. A method of treating Parkinson's disease comprising administering to a subject an effective amount of a compound of any one of claims 1-3, or the pharmaceutical composition of claim 4.

11. The method of claim 10, wherein the Parkinson's disease is LRRK2 mutation associated Parkinson's disease.
